(19) United States
(12) Patent Application Publication
(10) Pub. No.: US 2026/0097181 A1
(43) Pub. Date: Apr. 9, 2026

(54) GRADIENT ANNEALING OF POLYMERIC COMPONENTS

(71) Applicant: Access Vascular, Inc., Billerica, MA (US)

(72) Inventors: Daniel T. Donahue, Cambridge, MA (US); Michael Bassett, Hampton, NH (US); Matthew M. Mannarino, Burlington, MA (US)

(73) Assignee: Access Vascular, Inc., Billerica, MA (US)

(21) Appl. No.: 19/242,135

(22) Filed: Jun. 18, 2025

Related U.S. Application Data
(60) Provisional application No. 63/662,963, filed on Jun. 21, 2024.

(51) Int. Cl. A61M 25/00

(52) U.S. Cl. CPC A61M 25/0013; A61M 2202/0007; A61M 2205/36

(57) ABSTRACT

Articles and devices, such as catheters, comprising spatially varying physical properties are generally provided. The articles and devices provided herein may be configured to exhibit gradients in mechanical properties, e.g., as the result of spatially varying annealing using methods also described herein. In some embodiments, the articles (e.g., catheters) provided herein are useful in the medical arts. Advantages of the articles, devices, and methods generally provided herein include improved kink resistance of devices and articles comprising polymeric tubes, as well as circumvention of otherwise important compromises between patient comfort (e.g., which may be increased via use of soft polymeric tubes) and ease of insertion (e.g., which may be improved in stiffer polymeric tubes) by spatially varying properties of articles or devices based on their function.

### TECHNICAL FIELD

The present invention relates generally to devices and articles, such as catheters, comprising spatial variation in their physical properties. The devices and articles may be configured to be at least partially positioned within a patient, such as articles and/or devices that include polymeric tubes that are configured to be positioned in a blood vessel or other patient conduit.

### BACKGROUND

Devices and articles with high strength, low thrombogenicity, lubricious surface properties and containing a biologically active agent are generally useful in the medical arts. Different uses of medical devices and articles often benefit from different physical properties. The properties of materials can generally be improved with surface treatments so that the bulk material properties are preserved and the surface has properties that a preferable relative to the properties of the bulk material. However, despite such treatments, thrombus formation on medical devices may still restrict flow through and around the device, which can adversely affect infusion and aspiration, often requiring the use of expensive thrombolytic medications or even device replacement to resolve the blockage. For example, seconds after a catheter is placed into the bloodstream, blood proteins (e.g., fibrinogen and collagen) and host cells (e.g., platelets) begin to deposit on the device surface. Thrombus formation restricts flow through and around the device and can become friable and dislodge into the bloodstream, which has resulted in deep vein thrombosis and pulmonary embolism in several major clinical studies. Complications seen with such devices lengthen hospital stays and increase patient morbidity and mortality. Accordingly, improved devices and methods are needed

### SUMMARY

Gradient annealing of polymeric components and related articles and devices are generally provided. The subject matter of the present disclosure involves, in some cases, interrelated products, alternative solutions to a particular problem, and/or a plurality of different uses of one or more systems and/or articles.

In one aspect, an article is provided. In some embodiments, the article comprises: a polymeric tube comprising a lumen, a first portion and a second portion; wherein: the first portion at least partially disposed between the second portion and the lumen, the first portion has a first Young's elastic modulus, the second portion has a second Young's elastic modulus, and the first Young's elastic modulus is different than the second Young's elastic modulus, and wherein one or more of the following hold: (i) the polymeric tube comprises at least one water soluble polymer, (ii) the polymeric tube is free of covalent crosslinking agents, (iii) the polymeric tube has a solids content of 33% to 90% w/w at equilibrium water content (EWC), (iv) the first portion and/or the second portion has a Young's modulus of 5-100 MPa at equilibrium water content (EWC), and (v) the polymeric tube comprises a plurality of interconnected pores, the pores having an average diameter of less than or equal to 1 micron.

In another aspect, an article is provided. In some embodiments, the article comprises: a polymeric tube comprising a lumen, a first portion and a second portion; wherein: a thickness of the first portion perpendicular to the lumen differs from a thickness of the second portion perpendicular to the lumen, the first portion has a first Young's elastic modulus, the second portion has a second Young's elastic modulus, and the first Young's elastic modulus is different than the second Young's elastic modulus, and wherein one or more of the following hold: (i) the polymeric tube comprises at least one water soluble polymer, (ii) the polymeric tube is free of covalent crosslinking agents, (iii) the polymeric tube has a solids content of 33% to 90% w/w at equilibrium water content (EWC), (iv) the first portion and/or the second portion has a Young's modulus of 5-100 MPa at equilibrium water content (EWC), and (v) the polymeric tube comprises a plurality of interconnected pores, the pores having an average diameter of less than or equal to 1 micron.

In yet another aspect, an article is provided. In some embodiments, the article comprises: a polymeric tube comprising a lumen, a first portion and a second portion; wherein: (a) the first portion has a first density, the second portion has a second density, and the first density is different from the second density; (b) the first portion has a first anisotropy, the second portion has a second anisotropy, and the first anisotropy is different from the second anisotropy; and/or (c) the first portion has a first crystallinity, the second portion has a second crystallinity, and the first crystallinity is different from the second crystallinity, and wherein one or more of the following hold: (i) the polymeric tube comprises at least one water soluble polymer, (ii) the polymeric tube is free of covalent crosslinking agents, (iii) the polymeric tube has a solids content of 33% to 90% w/w at equilibrium water content (EWC), (iv) the first portion and/or the second portion has a Young's modulus of 5-100 MPa at equilibrium water content (EWC), and (v) the polymeric tube comprises a plurality of interconnected pores, the pores having an average diameter of less than or equal to 1 micron.

In still another aspect, an article is provided. In some embodiments, the article comprises: a polymeric tube comprising a lumen, a first portion and a second portion, wherein: the first portion has a first Young's elastic modulus, the second portion has a second Young's elastic modulus, and the first Young's elastic modulus is different than the second Young's elastic modulus, and wherein one or more of the following hold: (i) the polymeric tube comprises at least one water soluble polymer, (ii) the polymeric tube is free of covalent crosslinking agents, (iii) the polymeric tube has a solids content of 33% to 90% w/w at equilibrium water content (EWC), (iv) the first portion and/or the second portion has a Young's modulus of 5-100 MPa at equilibrium water content (EWC), and (v) the polymeric tube comprises a plurality of interconnected pores, the pores having an average diameter of less than or equal to 1 micron.

In another aspect, a method of forming a swellable article is provided. In some embodiments, the method comprises: with a polymeric mixture comprising at least one water soluble polymer and a solvent, the polymeric mixture having a concentration of at least 10% w/w of the at least one water soluble polymer, performing the steps of: heating the polymeric mixture to achieve a first temperature above the melting point of the polymeric mixture, extruding the polymeric mixture as a tube, and (a) inducing a thermal gradient in the tube to produce a gradient in a mechanical property of the tube; or (b) exposing a first portion of the tube to a first solvent for a duration such that a second portion of the tube is not exposed to the solvent for the duration to produce a gradient in a mechanical property of the tube.

In yet another aspect, a method of forming a swellable article is provided. In some embodiments, the method comprises: with a polymeric mixture comprising at least one water soluble polymer and a solvent, the polymeric mixture having a concentration of at least 10% w/w of the at least one water soluble polymer, performing the steps of: heating the polymeric mixture to achieve a first temperature above the melting point of the polymeric mixture, extruding the polymeric mixture as a tube, and dipping the tube at a first rate into a thermal reservoir at a reservoir temperature such that a first portion of the tube is exposed to the thermal reservoir at a first duration, different than a second duration of a second portion of the tube.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a perspective schematic diagram of an exemplary polymeric tube, according to one set of embodiments.

FIG. 2 is a cross-sectional schematic diagram of an exemplary polymeric tube, according to one set of embodiments.

FIG. 3 is a cross-sectional schematic diagram of an exemplary polymeric tube, according to one set of embodiments.

FIG. 4 is a cross-sectional schematic diagram of an exemplary polymeric tube, according to one set of embodiments.

FIG. 5A is a cross-sectional schematic diagram of an exemplary device, according to one set of embodiments.

FIG. 5B is a cross-sectional schematic diagram of an exemplary device comprising a plurality of pores, according to one set of embodiments.

FIG. 5C is a cross-sectional schematic diagram of an exemplary device comprising a plurality of pores, according to one set of embodiments.

FIG. 5D is a schematic of an exemplary extrusion apparatus to form a continuous form with a cut-away view of a side of the bath, according to one set of embodiments.

FIG. 5E is an enlarged view of a portion of the apparatus of FIG. 5D depicting the die head in perspective as viewed from the outside of the bath, according to one set of embodiments.

FIG. 5F is an enlarged view of a portion of the apparatus of FIG. 5D depicting the die head as disposed in the bath, according to one set of embodiments.

FIG. 5G is a cross-sectional schematic diagram of an exemplary device, according to one set of embodiments.

FIG. 6 is a side view of a catheter depicting the change in dimensions before and after swelling, according to one set of embodiments.

FIG. 7A is a schematic of a process of bulk incorporation of a polymer into a porous solid, according to one set of embodiments.

FIG. 7B is a cross-section of a portion of a tube taken along line 7B-7B of FIG. 7A, according to one set of embodiments.

### DETAILED DESCRIPTION

Articles with spatially varying, high strength porous materials incorporating water soluble polymers, as well as associated methods, kits, and devices, are generally provided. Materials, methods, and uses set forth herein may relate to biomaterials comprising a medically acceptable porous solid. Disclosed compositions and devices may be useful for administration to a subject (e.g., a patient). As a particular example, spatial variation in the properties of compositions and devices provided herein may advantageously facilitate mechanical reinforcement of various properties in device portions that benefit from high strength (e.g., portions of a device prone to kinking, or which portions of a device that are configured for rigid insertion into a subject). Advantageously, the compositions and/or devices described herein may be substantially non-thrombogenic, lubricious, and/or biocompatible. In some embodiments, the devices described herein may be useful for the delivery of a biologically active agent (e.g., a therapeutic agent such as a drug) to a subject. In some embodiments, the compositions and/or devices described herein may be suitable for administration to a subject and/or delivery of a biologically active agent for a relatively long period of time, e.g., without the formation of a thrombus, without fouling, and/or without absorbing (or adsorbing) one or more substances (e.g., therapeutic agents, proteins, blood, plasma) internal to the subject. Methods for forming such compositions and/or devices are also provided.

The devices described herein may be used, in some cases, to make blood-contacting devices or devices that contact bodily fluids, including ex vivo and/or in vivo devices, such as blood contacting implants. Examples of drug delivery devices in which the devices described herein may embody or be incorporated into include but are not limited to medical tubing, wound dressing, contraceptive devices, feminine hygiene, endoscopes, grafts (e.g., including small diameter of less than or equal to 6 mm), pacemakers, implantable cardioverter-defibrillators, cardiac resynchronization devices, cardiovascular device leads, ventricular assist devices, catheters (e.g., including cochlear implants, endotracheal tubes, tracheostomy tubes, ports, shunts), implantable sensors (e.g., intravascular, transdermal, intracranial), ventilator pumps, and ophthalmic devices including drug delivery systems.

Particular advantages have been noted to the spatial variation of properties within articles or devices comprising polymeric tubes, according to some embodiments. In some embodiments, the disclosure relates to articles comprising a polymeric tube that comprises a lumen and a plurality of portions including a first portion and a second portion. The first portion and the second portion may be discrete portions of the polymeric tube (e.g., portions that do not include volumetric overlap with one another). In some embodiments, the portions have different physical properties, as discussed in greater detail below.

The lumen may traverse the length of the polymeric tube (e.g., such that it opens at both ends of the polymeric tube). The lumen may open at one or more (e.g., both) ends of the polymeric tube. For example, in some embodiments, the lumen is open at both ends. According to some embodiments, the lumen is capped at one or more end (e.g., for polymeric tubes of external ventricular drain devices). In some embodiments, the lumen passes through the center of the polymeric tube. However, the lumen may be offset from the center of the polymeric tube as the disclosure is not so limited. For example, in some embodiments, the polymeric tube could comprise a plurality of lumens, as the disclosure is not limited to embodiments of polymeric tubes containing exactly one lumen. In some embodiments, the lumen traverses at least a portion of the length of the polymeric tube.

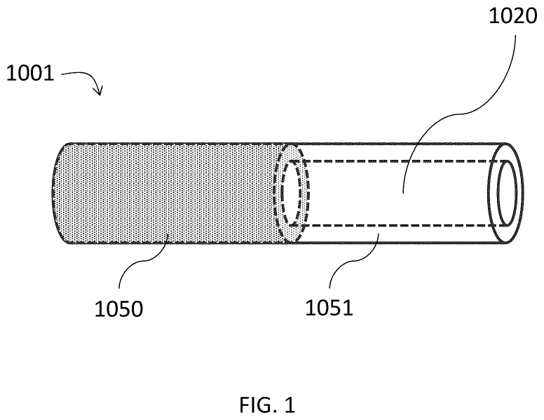

FIG. 1 provides a non-limiting perspective illustration of a polymeric tube 1001 comprising lumen 1020. The polymeric tube may have any of a variety of suitable transverse cross-sections (e.g., cross-sections perpendicular to a lumen of the polymeric tube). For example, as shown in FIG. 1, polymeric tube 1001 is cylindrical and has a circular (annular) transverse cross-section. More generally, the polymeric tube may have a square, polygonal, circular, or elliptical transverse cross-section, or may have any of a variety of other transverse cross-sections, as the disclosure is not so limited. Likewise, the lumen may have any of a variety of suitable transverse cross-sections. For example, as shown in FIG. 1, lumen 1020 is cylindrical and has a circular transverse cross-section. More generally, the lumen may have a square, polygonal, circular, or elliptical transverse cross-section, or may have any of a variety of other transverse cross-sections, as the disclosure is not so limited.

One advantage of the polymeric tubes provided herein is that they may have spatial variation in physical properties. As detailed below, the present disclosure generally relates, in some embodiments, to polymeric materials with advantageous properties for medical articles and devices. The spatial variation of physical properties of these polymeric materials within the polymeric tube may, for example, allow different portions of the polymeric tube to have different properties better tailored to their individual functions within an article or device while allowing the entire polymeric tube to retain advantages associated with the polymeric tube's constituent materials. To provide a specific, non-limiting illustration, in some embodiments an end portion of a polymeric tube may have a higher stiffness than a central portion of the polymeric tube to facilitate insertion of the end portion into a subject. Polymeric tubes with stiffened end portions may be advantageous, e.g., in the context of catheters, where the stiffened end portion may be used to help insert the catheter (e.g., to help align an end of the catheter and/or to help puncture the catheter through tissue such as the ventricles, heart, veins, arteries, or peritoneal cavity), but where flexibility in the remainder of the catheter may be preferred for the comfort of a subject. For example, polymeric tubes with stiffened end portions may be useful for placement of valves, shunts, fistulas, and catheters, depending on the embodiment. These, and a number of other applications are discussed in greater detail below.

A polymeric tube with spatial variation in physical properties may comprise a plurality of portions (e.g., two, three, four, five, or more portions) each with different properties. The first portion and the second portion (and optionally, one or more additional portions) of the polymeric tube may have different physical properties. Moreover, within any given portion of the polymeric tube, physical properties may be homogeneous (e.g., in the case where the portion has substantially constant physical properties regardless of spatial position) or heterogeneous (e.g., where one or more physical properties varies as a function of spatial position within the portion), depending on the embodiment. For example, in some embodiments, a first portion may have a first set of physical properties that are homogeneous; a second portion may have a second set of physical properties; and a third portion connecting the first portion to the second portion, wherein the third portion has a heterogeneous gradient of physical properties as the physical properties vary continuously from the properties of the first portion to the properties of the second portion. Of course, it should be understood that other apportionments of the polymeric tube are also possible, and that the preceding example is merely illustrative.

Any of a variety of physical properties may spatially vary within the polymeric tube. A few, non-limiting examples of physical properties that may spatially vary include, but are not limited to: mechanical properties (e.g., Young's elastic modulus, shear modulus, bulk modulus, Poisson ratio, and/or, for anisotropic polymers, stiffness and/or compliance tensor coefficients), anisotropy, crystallinity, pore properties (e.g., porosity, pore size), and density. Spatial variation of one or more of these properties may have the result that the polymeric tube comprises a first portion having a first value of any one of these physical properties and a second portion having a second value of an one of these physical properties, wherein the first value differs from the second value. For example, in some embodiments, a polymeric tube comprises a first portion having a first Young's elastic modulus and a second portion having a second Young's elastic modulus, where the first Young's elastic modulus has a different value than the second Young's elastic modulus. As another example, in some embodiments, a polymeric tube comprises a first portion having a first density and a second portion having a second density, where the first density is different from the second density. As still another example, in some embodiments, a polymeric tube comprises a first portion having a first anisotropy and a second portion having a second anisotropy, where the first anisotropy is different from the second anisotropy. As still another example, in some embodiments, a polymeric tube comprises a first portion having a first crystallinity and a second portion having a second crystallinity, where the first crystallinity is different from the second crystallinity.

Referring again to FIG. 1, polymeric tube 1001 is an example of a polymeric tube with spatial variation in physical properties. For example, first portion 1050 of polymeric tube 1001 as illustrated has a first value of a physical property (e.g., a first value of the Young's elastic modulus) and second portion 1051 of polymeric tube 1001 has a second value of the physical property (e.g., a second value of Young's elastic modulus) differing from the first value of the physical property associated with the first portion of the polymeric tube. The first portion and

Portions of the polymeric tube (e.g., the first portion, the second portion) may have any of a variety of appropriate geometries. In some embodiments, the first portion and the second portion do not overlap along the length of the lumen, e.g., because they are offset along the length of the lumen such that no line perpendicular to the lumen passes through both the first portion and the second portion. For example, in some embodiments the first portion and/or the second portion comprise the entire thickness of the polymeric tube perpendicular to the lumen, but are offset relative to one another along axis of polymeric tube. FIG. 1 illustrates just such an example, since portions 1050 and 1051 both occupy the entire transverse cross-section of the tube but are offset from one another along the tube's length. In some embodiments, the first portion and the second portion may overlap along at least a part of the length of the lumen, e.g., such that at least one line perpendicular to the lumen passes through both the first portion and the second portion. Thus, in at least some embodiments, a polymeric tube comprises a first portion at least partially disposed between a second portion and a lumen of the polymeric tube. As a special case, for example, portions of a polymeric tube may completely overlap such that a second portion of the polymeric tube is completely separated from a the lumen by a first portion of the polymeric tube disposed between the lumen and the second portion.

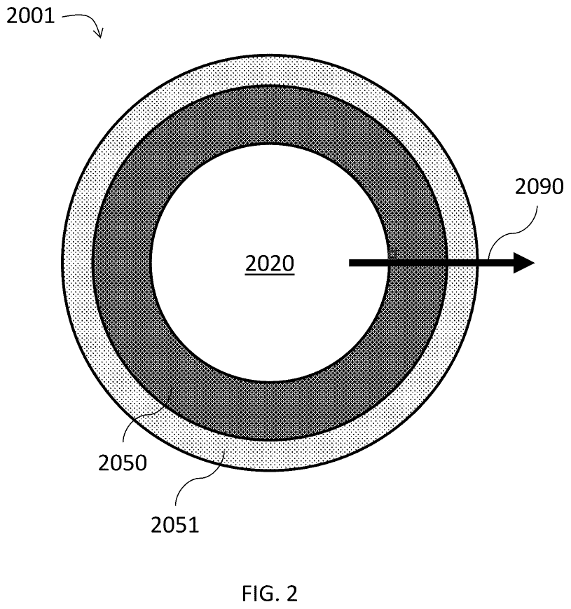

FIG. 2 presents a non-limiting schematic cross-section of a polymeric tube 2001 transverse to lumen 2020, showing that polymeric tube 2001 comprises a first portion 2050 having a first value of a physical property and a second portion 2051 having a second value of the physical property, where the first value of the physical property and the second value of the physical property are different and wherein first portion 2050 separates lumen 2020 from second portion 2051 such that first portion 2050 and second portion 2051 overlap at line 2090 perpendicular to lumen 2020 (which extends perpendicularly from the plane of the figure).

A polymeric tube may have any of a variety of suitable dimensions, depending on the embodiment. For example, in some embodiments, a polymeric tube has a length (e.g., parallel to a lumen of the tube) of greater than or equal to 10 cm, greater than or equal to 20 cm, greater than or equal to 30 cm, greater than or equal to 40 cm, greater than or equal to 50 cm, greater than or equal to 60 cm, greater than or equal to 70 cm, greater than or equal to 80 cm, greater than or equal to 90 cm, greater than or equal to 100 cm, greater than or equal to 110 cm, greater than or equal to 120 cm, greater than or equal to 130 cm, greater than or equal to 140 cm, greater than or equal to 150 cm, greater than or equal to 160 cm, greater than or equal to 170 cm, greater than or equal to 180 cm, or greater than or equal to 190 cm. In some embodiments, a polymeric tube has a length (e.g., parallel to a lumen of the tube) of less than or equal to 200 cm, less than or equal to 190 cm, less than or equal to 180 cm, less than or equal to 170 cm, less than or equal to 160 cm, less than or equal to 150 cm, less than or equal to 140 cm, less than or equal to 130 cm, less than or equal to 120 cm, less than or equal to 110 cm, less than or equal to 100 cm, less than or equal to 90 cm, less than or equal to 80 cm, less than or equal to 70 cm, less than or equal to 60 cm, less than or equal to 50 cm, less than or equal to 40 cm, less than or equal to 30 cm, or less than or equal to 20 cm. Combinations of these ranges are also possible (e.g., greater than or equal to 10 cm and less than or equal to 200 cm, or greater than or equal to 10 cm and less than or equal to 100 cm). Other ranges, both higher and lower than those described above, are also possible, as the disclosure is not so limited.

A polymeric tube may have any of a variety of suitable aspect ratios. In some embodiments, the aspect ratio of the polymeric tube is the aspect ratio of the length of the polymeric tube (e.g., parallel to a lumen) to a maximum transverse dimension of the polymeric tube (e.g., perpendicular to a lumen) of the polymeric tube. In some embodiments, a polymeric tube has an aspect ratio of greater than or equal to 10:1, greater than or equal to 50:1, greater than or equal to 100:1, greater than or equal to 200:1, greater than or equal to 300:1, greater than or equal to 400:1, greater than or equal to 500:1, greater than or equal to 600:1, greater than or equal to 700:1, greater than or equal to 800:1, or greater than or equal to 900:1. In some embodiments, a polymeric tube has an aspect ratio of less than or equal to 1000:1, less than or equal to 900:1, less than or equal to 800:1, less than or equal to 700:1, less than or equal to 600:1, less than or equal to 500:1, less than or equal to 400:1, less than or equal to 300:1, less than or equal to 200:1, less than or equal to 100:1, or less than or equal to 50:1. Combinations of these ranges are also possible (e.g., greater than or equal to 10:1 and less than or equal to 1000:1, or greater than or equal to 50:1 and less than or equal to 500:1). Other ranges, both higher and lower than those described above, are also possible, as the disclosure is not so limited.

A polymeric tube may have uniform or variable transverse dimensions, depending on the embodiment. Likewise, a portion (e.g., a first portion, a second portion) of a polymeric tube may have uniform or variable transverse dimensions, depending on the embodiment. For example, FIGS. 1-2 show examples of polymeric tubes with uniform transverse dimensions, where the shape and dimension of the transverse cross-section do not change along the length of the lumen. Polymeric tubes with variable transverse dimensions may have dimensions that vary in any of a variety of ways. For example, in some embodiments, a polymeric tube has a transverse dimension that tapers (e.g., to a coned tip). Variation in a transverse dimension may sometimes be coupled with spatial variation in physical properties of a polymeric tube (e.g., a tapered variation in transverse dimensions may be coupled with a tapered variation in mechanical properties so that thinner portions of the tube are stiffer than thicker portions).

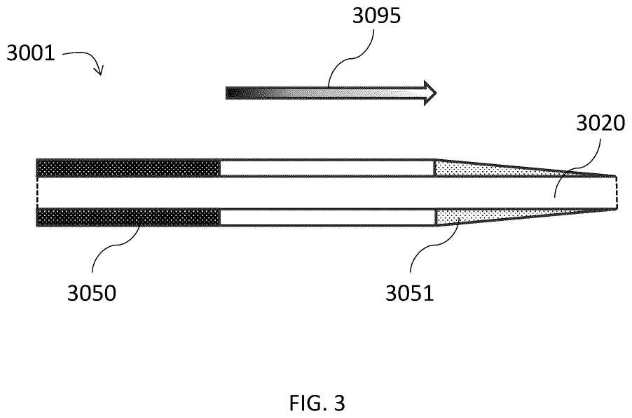

FIG. 3 provides an example of a polymeric tube with a variable transverse cross-section, schematizing a cross-section of a polymeric tube 3001 parallel to lumen 3020 (such that the height of the polymeric tube shown in the page is a transverse dimension of the polymeric tube). Polymeric tube 3001 comprises first portion 3050, which has a uniform cross section across its length, and second portion 3051 which has a variable transverse cross-section across the length of the lumen, as shown. As illustrated by this example, the thickness of the first portion may differ from the thickness of the second portion perpendicular to a lumen of the polymeric tube. For example, portion 3051 is thinner, on average, than portion 3050 perpendicular to lumen 3020. Such configurations may be advantageous, in some embodiments, when the thinner portion is an end portion of the polymeric tube 3001. For example, second portion 3051 of polymeric tube 3001 may be advantageous for insertion into a subject, e.g., since the narrow end may help align the tube with an orifice or other opening of the subject.

Likewise, portions of polymeric tubes may have any of a variety of appropriate dimensions. As contrasting FIG. 1 with FIGS. 2-3 demonstrates, in some embodiments portions of polymeric tubes have identical transverse cross-sections (e.g., portions 1050 and 1051 of FIG. 1) and in some embodiments portions of polymeric tubes have different transverse cross-sections (e.g., portions 2050 and 2051 of FIG. 2, or portions 3050 and 3051 of FIG. 3). Similarly, like the polymeric tube as a whole, portions of the polymeric tube may have uniform or variable transverse cross-sections. For example, as contrasting FIGS. 1-2 with FIG. 3 demonstrates, in some embodiments a portion of a polymeric tube has a uniform transverse cross-section (e.g., portions 1050 and 1051 of FIG. 1, or portions 2050 and 2051 of FIG. 2) and in some embodiments a portion of a polymeric tube has a variable transverse cross-section (e.g., portion 3051 but not portion 3050 of FIG. 3).

It may be particularly advantageous to mechanically reinforce thinner portions of the polymeric tube, according to some embodiments, by providing them with different mechanical properties relative to thicker portions of the polymeric tube. In some embodiments, a polymeric tube comprises a first portion and a second portion, and a thickness of the first portion perpendicular to the lumen differs from a thickness of the second portion perpendicular to the lumen. According to some such embodiments, the thickness of the first portion exceeds the thickness of the second portion and the Young's elastic modulus of the first portion is less than the Young's elastic modulus of the second portion. Such a configuration may provide added support for the thinner, second portion. For example, it may be advantageous to make a thinner portion of a polymeric tube stiffer by providing it with a higher Young's elastic modulus. For example, in FIG. 3, portion 3051 is thinner than portion 3050, on average, but portion 3051 may have a higher Young's elastic modulus than portion 3050.

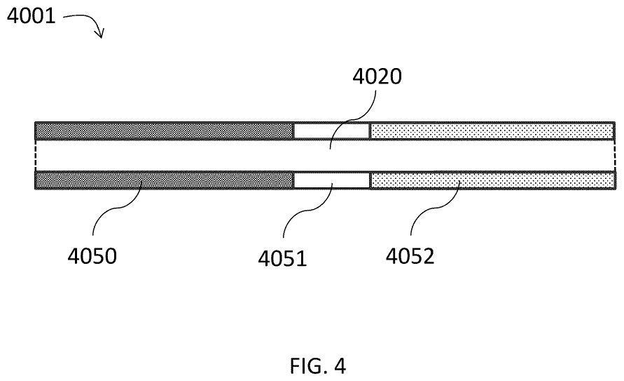

Another application of spatially varying the mechanical properties of a polymeric tube is reinforcement of portions of the polymeric tube that are prone to kinking, according to some embodiments. For example, in some embodiments, an article or device comprises a polymeric tube comprising a reinforced, second portion disposed between a first portion and a third portion. FIG. 4 provides one such example, providing a cross-sectional schematic illustration of a polymeric tube 4001 comprising lumen 4020, first portion 4050, and third portion 4052, separated by second portion 4051. A portion of the polymeric tube may be reinforced by adding thickness, varying mechanical properties, or both. For example, in some embodiments, a second portion disposed between the first portion and the third portion has a thickness exceeding the thickness of the first portion and the third portion. As another example, in some embodiments, the first portion, second portion, and third portion of a polymeric tube have a first, second, and third elastic modulus, respectively, and the second elastic modulus exceeds the first elastic modulus of the first portion and the second elastic modulus of the third portion. Other embodiments are also possible. For example, in embodiments it may be advantageous to vary physical properties of a polymeric tube along a spatial gradient, e.g., such that a third portion has a higher elastic modulus than a first portion or a second portion positioned between the first portion and the third portion.

A portion (e.g., a first portion, a second portion) of a polymeric tube provided herein may have any of a variety of suitable lengths parallel to an axis of the lumen. In principle, a thin tube could be continuously extruded without limitation as to length. Accordingly, it should be understood that while the lengths below are representative of portions of tubes according to various embodiments, there is no theoretical upper limit to the length of any given portion, and the disclosure is not so limited. In some embodiments, a portion (e.g., a first portion, a second portion) has a length of greater than or equal to 0.5 cm, greater than or equal to 1 cm, greater than or equal to 1.5 cm, greater than or equal to 2 cm, greater than or equal to 2.5 cm, greater than or equal to 3 cm, greater than or equal to 3.5 cm, greater than or equal to 4 cm, or greater than or equal to 4.5 cm. In some embodiments, a portion (e.g., a first portion, a second portion) has a length of less than or equal to 5 cm, less than or equal to 4.5 cm, less than or equal to 4 cm, less than or equal to 3.5 cm, less than or equal to 3 cm, less than or equal to 2.5 cm, less than or equal to 2 cm, less than or equal to 1.5 cm, or less than or equal to 1 cm. Combinations of these ranges are also possible (e.g., greater than or equal to 0.5 cm and less than or equal to 5 cm, or greater than or equal to 1 cm and less than or equal to 4 cm). Other ranges, both higher and lower than those described above, are also possible, as the disclosure is not so limited. It should, of course, be understood that where the polymeric tube comprises a plurality of portions, the lengths in the above-mentioned ranges may each, independently, be appropriate for describing any of the portions of the plurality. For example, the length of a first portion and a second portion, and optionally a third, fourth, fifth, etc. portion may each, independently, be selected from within one or more of the above-mentioned ranges.

A portion (e.g., a first portion, a second portion) of a polymeric tube provided herein may have any of a variety of suitable average thicknesses perpendicular to an axis of the lumen. In some embodiments, a portion (e.g., a first portion, a second portion) has an average thickness of greater than or equal to 5 microns, greater than or equal to 10microns, greater than or equal to 50 microns, greater than or equal to 0.1 mm, greater than or equal to 0.5 mm, greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm, greater than or equal to 11 mm, greater than or equal to 12 mm, greater than or equal to 13 mm, or greater than or equal to 14 mm. In some embodiments, a portion (e.g., a first portion, a second portion) has an average thickness of less than or equal to 15 mm, less than or equal to 14 mm, less than or equal to 13 mm, less than or equal to 12 mm, less than or equal to 11 mm, less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, less than or equal to 2 mm, less than or equal to 1 mm, less than or equal to 0.5 mm, less than or equal to 0.1 mm, less than or equal to 50 microns, or less than or equal to 10 microns. Combinations of these ranges are also possible (e.g., greater than or equal to 5 microns and less than or equal to 15 mm, greater than or equal to 0.1 mm and less than or equal to 5 mm, or greater than or equal to 0.5 mm and less than or equal to 5 mm). Other ranges, both higher and lower than those described above, are also possible, as the disclosure is not so limited. It should, of course, be understood that where the polymeric tube comprises a plurality of portions, the average thicknesses in the above-mentioned ranges may each, independently, be appropriate for describing any of the portions of the plurality. For example, the average thickness of a first portion and a second portion, and optionally a third, fourth, fifth, etc. portion may each, independently, be selected from within one or more of the above-mentioned ranges.

In an exemplary set of embodiments, a device comprising a polymeric tube is a catheter. In some embodiments, the catheter is configured for administration to a subject. For example, in some embodiments, a catheter is formed of a polymeric material and is configured for administration to a subject, wherein the catheter comprises a biologically active agent distributed within the polymeric material (e.g., distributed homogeneously). In some embodiments, the catheter comprises a body portion, wherein the body portion is formed of a polymeric material comprising a first water soluble polymer, as described herein.

In a device or article, various portions described above may have any of a variety of appropriate relative positions. For example, in some embodiments, a second portion (e.g., portion 3051 of FIG. 3) may be a proximal portion of polymeric tube relative to the first portion (where “proximal” is relative to a subject connected to the device or article). Likewise, the second portion may be a distal portion of the polymeric tube relative to the first portion. And, of course, in some embodiments the first portion are both equally proximal and distal (e.g., in the case where the first portion and the second portion overlap each other along the length of the lumen, as in polymeric tube 3001 of FIG. 3). And in some embodiments, the second portion is internal (where “internal” is relative to the center of a lumen of the polymeric tube) relative to the first portion, while in others, the first portion is internal relative to the second portion. Where the polymeric tube comprises one or more additional portions (e.g., a third portion, a fourth portion, a fifth portion, etc.), these additional portions may have any of a variety of appropriate positions relative to the first portion and the second portion.

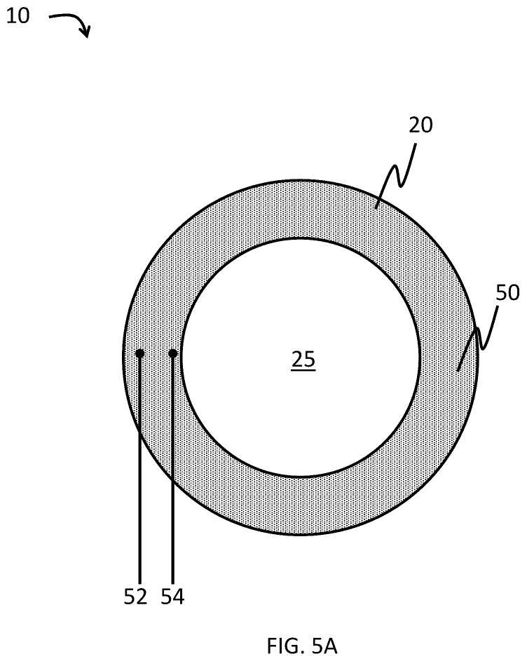

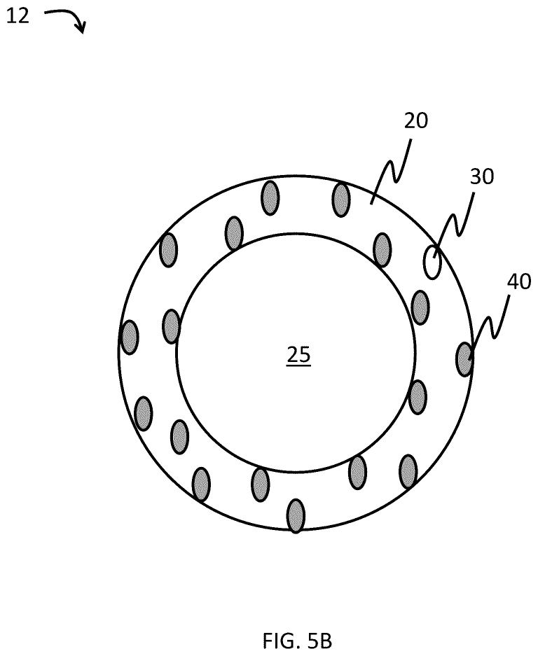

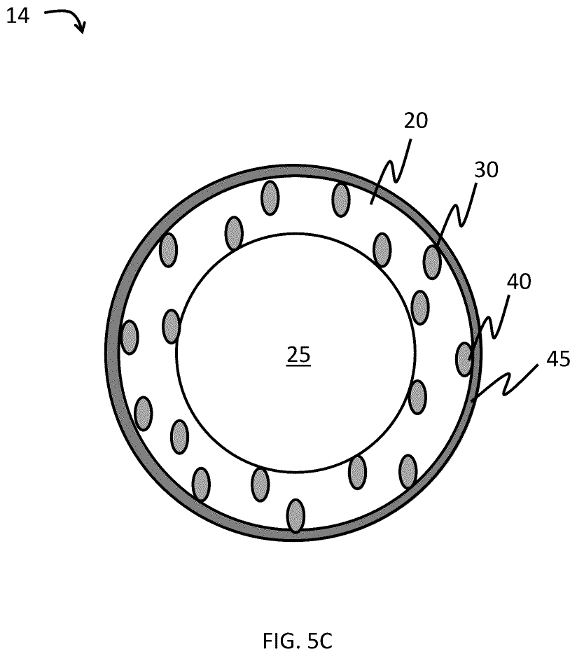

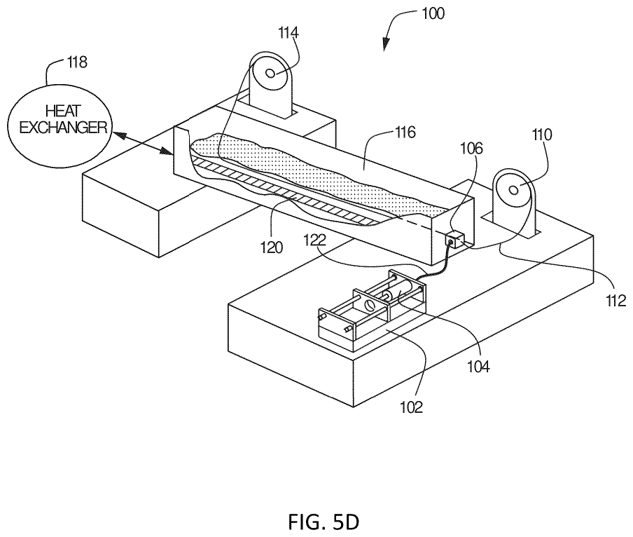

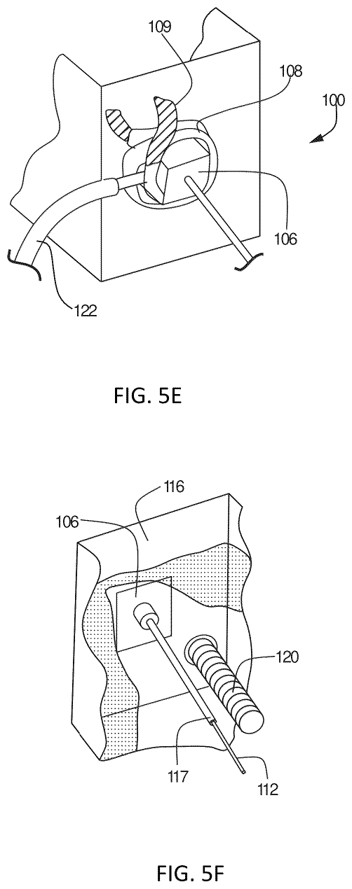

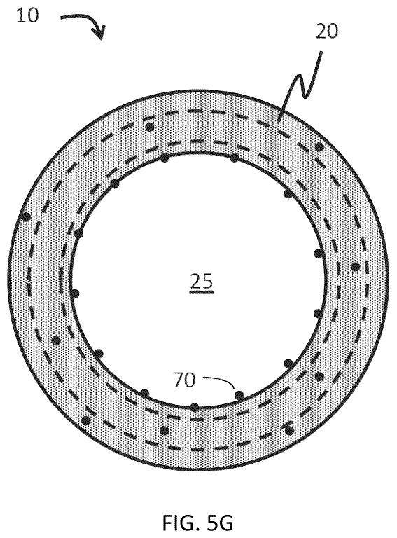

In some embodiments, the devices described herein comprise a body portion. For example, as shown illustratively in FIG. 5A, device 10 comprises a body portion 20 (e.g., which may comprise the first portion, second portion, etc., of the polymeric tube). In some embodiments, body portion 20 is formed of and/or comprises a polymeric material. The polymeric material may comprise a first water soluble polymer. In some embodiments a biologically active agent 50 is associated with the polymeric material. In some embodiments, the device is a polymeric tube (e.g., a catheter) although other shapes and/or configurations are also possible.

In some embodiments, one or more biologically active agents is present throughout the bulk of the polymeric material (e.g., distributed throughout the polymeric material matrix). For example, in some embodiments, a first arbitrary section 52 within a cross-section of body portion 20 comprises a non-zero concentration of a biologically active agent. In some embodiments, a second arbitrary section 54, different than first arbitrary section 52, within a cross-section of body portion 20 comprises a non-zero concentration of the biologically active agent. Those of ordinary skill in the art would understand, based upon the teachings of this specification, that the presence of a biologically active agent within the bulk of a polymeric material (e.g., embedded in the polymer matrix of the polymeric material) is not intended to refer to a coating of a biologically active agent on a polymeric material but, by contrast, is intended to refer to a biologically active agent distributed throughout the bulk of the polymeric material. However, in some embodiments, a coating comprises the biologically active agent may optionally be present. Examples of sections are described in more detail below.

While body portion 20, section 52 and section 54 in FIG. 5A are depicted as circular, those of ordinary skill in the art would understand, based upon the teachings of this specification, that the body portion, like the first and second portions described above, need not be circular, and that other cross-sectional shapes (e.g., planar, rectangular, square, oval, oblong, S-shaped, etc.) are also possible. For example, in some embodiments, the body portion is S-shaped, which can, in some cases, provide ease of implantation in a subject, achieve lower infiltration rates, and reduce the likelihood of dislodgement within the subject.

In some embodiments, the tubes (e.g., polymeric tube 10 of FIG. 5A, polymeric tube 12 of FIG. 5B, polymeric tube 14 of FIG. 5C) described herein comprise a body portion having a plurality of pores. The body portion may be formed of a polymeric material comprising a first water soluble polymer. In some embodiments, the body portion further comprises a second water soluble polymer, same or different than the first water soluble polymer. For example, in some embodiments, a second water soluble polymer, same or different than the first water soluble polymer, may be positioned within at least a portion of the plurality of pores. In some embodiments, the second water soluble polymer is positioned within the bulk of the first water soluble polymer. In some embodiments, the second water soluble polymer is substantially homogeneously dispersed within the bulk of the first water soluble polymer. In some embodiments, the second water soluble polymer is substantially non-homogeneously dispersed within the bulk of the first water soluble polymer. While the following embodiments may refer to polymeric tubes comprising a second water soluble polymer positioned within the plurality of pores, those of ordinary skill in the art would understand, based upon the teachings of this specification, that a second water soluble polymer need not always be present. Without wishing to be bound by theory, in some embodiments, the presence of a second water soluble polymer positioned within at least a portion of the plurality of the pores of the body portion or first water soluble polymer may decrease the thrombogenicity and/or increase the lubriciousness of the polymeric tube (e.g., polymeric tube 12 of FIG. 5B, polymeric tube 14 of FIB. 5C) as compared to polymeric tubes without the second water soluble polymer positioned within the pores (all other factors being equal). In an exemplary set of embodiments, the first water soluble polymer is polyvinyl alcohol. In another exemplary set of embodiments, the second water soluble polymer is polyacrylic acid. Other water soluble polymers are also possible, as described herein.

In some embodiments, the second water soluble polymer may be considered the same as the first water soluble polymer when they are both polymers of the same monomer(s) but have other characteristics, such as the number of monomer(s) and/or molecular weight, that differ.

In some embodiments, articles or devices comprising a polymeric tube as described herein (e.g., polymeric tube 10 of FIG. 5A, polymeric tube 12 of FIG. 5B, polymeric tube 14 of FIG. 5C) and compositions described herein are administered to a subject. In some embodiments, the article or device may be administered orally, rectally, vaginally, nasally, intravenously, subcutaneously, or uretherally. In some cases, the article or device may be administered into a cavity, epidural space, vein, artery, orifice, external orifice, and/or abscess of a subject. A non-limiting example of an orifice includes a wound. A non-limiting example of a wound includes a wound orifice that is created for venous access (e.g., created as an insertion site) through the skin.

As described herein, in some embodiments, the compositions, articles, polymeric tubes, and/or devices described herein comprise or are formed of a polymeric material comprising a first water soluble polymer having a plurality of pores. For example, as illustrated in FIG. 5B polymeric tube 12 includes a body portion 20 comprising or formed of a polymeric material comprising a first water soluble polymer and having a plurality of pores 30. In some embodiments, second water soluble polymer 40 is positioned within at least a portion (e.g., at least 10%, at least 20%, at least 30%, at least 40%, at least 50%, at least 60%, at least 70%, at least 80%, at least 90%, at least 95%, at least 98%, at least 99%, at least 99.99%) of the plurality of pores. In some embodiments, second water soluble polymer 40 is positioned within less than or equal to 100%, less than or equal to 90%, less than or equal to 80%, less than or equal to 70%, less than or equal to 60%, less than or equal to 50%, less than or equal to 40%, less than or equal to 30%, less than or equal to 20%, or less than or equal to 10% of the plurality of pores (e.g., at least 10% and less than or equal to 100% of the plurality of pores). Combinations of the above-referenced ranges are also possible.

In some embodiments, the second water soluble polymer is positioned within (e.g., dispersed within) the bulk of the first water soluble polymer (e.g., within the pores and/or interstices of the first water soluble polymer). In some embodiments, as illustrated in FIG. 5C, the second water soluble polymer 40 may be present as a coating 45 on at least a portion of a surface of body portion 20. Although FIG. 5C shows the second water soluble polymer as a coating on the first water soluble polymer and in the pores of the first water soluble polymer, it should be appreciated that in some embodiments, only a coating 45 is present and the pores 30 are not substantially filled with the second water soluble polymer 40. Other configurations are also possible.

In some embodiments, the polymeric tubes described herein may be hollow (e.g., have a hollow core such as a lumen). For example, polymeric tube 10 and/or polymeric tube 12 may be hollow (e.g., comprising a lumen 25). However, while FIGS. 5A-5C are depicted having a lumen, those of ordinary skill in the art would understand based upon the teachings of this specification that such a lumen may not be present. That is to say, in some cases, the lumen 25 of the (e.g., polymeric tube 10 of FIG. 5A, polymeric tube 12 of FIG. 5B, polymeric tube 14 of FIG. 5C) may be a bulk material (e.g., a solid core) without a lumen 25 or other hollow core.

In some embodiments, the polymeric tube (e.g., polymeric tube 10 of FIG. 5A, polymeric tube 12 of FIG. 5B, polymeric tube 14 of FIG. 5C) described herein are, are comprised by, or are configured for use with, a medical device such as a catheter, a balloon, a shunt, a wound drain, an infusion port, a drug delivery device, a tube, a contraceptive device, a feminine hygiene device, an endoscope, a graft, a pacemaker, an implantable cardioverter-defibrillator, a cardiac resynchronization device, a cardiovascular device lead, a ventricular assist device, an endotracheal tube, a tracheostomy tube, an implantable sensor, a ventilator pump, and an ophthalmic device. In some embodiments, the catheter is selected from the group consisting of hemodialysis catheters, central venous catheters, peripheral central catheters, peripherally inserted central catheters (PICCs), midline catheters, peripheral catheters, tunneled catheters, dialysis access catheters, urinary catheters, neurological catheters, percutaneous transluminal angioplasty catheters and/or peritoneal catheters. Other suitable uses are described in more detail, below.

Spatial variation in physical properties may take any of a variety of suitable forms within a polymeric tube. The spatial variation may be associated with a gradient of the varying physical properties. Generally, a gradient in a physical property may have any of a variety of orientations within the polymeric material forming the polymeric tube. For example, in some embodiments, spatial variation gives rise to a gradient of a physical property in a cross-sectional dimension of the polymeric tube. In some embodiments, for example, the polymeric tube has a gradient in a physical property (e.g., Young's elastic modulus) along the length of the polymeric tube parallel to the lumen. As another example, in some embodiments, the polymeric tube has a gradient in a physical property in a transverse dimension of the tube (e.g., a dimension perpendicular to a lumen of the tube, such as along a diameter of the polymeric tube).

In some embodiments, a gradient of a physical property is a continuous gradient, e.g., where the property changes continuously along a length of greater than 1 mm, greater than or equal to 3 mm, 5 mm, 10 mm, 15 mm, 20 mm, 50 mm, or more of a polymeric tube. For example, referring again to FIG. 3, polymeric tube 3001 has a continuous gradient in one or more physical properties (E.g., Young's elastic modulus), indicated by gradient arrow 3095, extending between first portion 3050 and second portion 3051. In some embodiments, a gradient of a physical property is discontinuous, e.g., such that an at least 10% change in the value of a physical property occurs along a length of less or equal to than 1 mm, less than or equal to 0.5 mm, less than or equal to 0.1 mm, less than or equal to 0.05 mm, less than or equal to 0.01 mm, or less. For example, referring again to FIG. 1, since first portion 1050 and second portion 1051 have distinct physical properties but are immediately adjacent, there is a discontinuous gradient of physical properties at the interface between portions 1050 and 1051. Different gradients in physical properties may be established by different methods, as discussed in greater detail below. Furthermore, it should be understood that a polymeric tube may comprise a plurality of gradients of physical properties, and that multiple gradients within a polymeric tube may be established independently, depending on the embodiment.

A gradient in a physical property may have any of a variety of suitable magnitudes. In some embodiments, a value associated with a physical property (e.g., a Young's elastic modulus, an anisotropy, a density, a crystallinity, a porosity) has an average gradient (in terms of a percent change in the value per centimeter) of greater than or equal to 1%/cm, greater than or equal to 5%/cm, greater than or equal to 10 %/cm, greater than or equal to 25%/cm, greater than or equal to 50%/cm, greater than or equal to 75%/cm, greater than or equal to 100%/cm, greater than or equal to 150%/cm, greater than or equal to 200%/cm, greater than or equal to 250%/cm, greater than or equal to 300%/cm, greater than or equal to 350%/cm, greater than or equal to 400 %/cm, greater than or equal to 450%/cm, greater than or equal to 500%/cm, greater than or equal to 550%/cm, greater than or equal to 600%/cm, greater than or equal to 800 %/cm, greater than or equal to 1000%/cm, or greater than or equal to 2000%/cm. In some embodiments, a value associated with a physical property has an average gradient (in terms of a percent change in the value per centimeter) of less than or equal to 5000 %/cm, less than or equal to 2000%/cm, less than or equal to 1000%/cm, less than or equal to 800%/cm, less than or equal to 600%/cm, less than or equal to 550%/cm, less than or equal to 500%/cm, less than or equal to 450%/cm, less than or equal to 400 %/cm, less than or equal to 350%/cm, less than or equal to 300%/cm, less than or equal to 250%/cm, less than or equal to 200%/cm, less than or equal to 150%/cm, less than or equal to 100%/cm, less than or equal to 75%/cm, less than or equal to 50%/cm, less than or equal to 25%/cm, less than or equal to 10%/cm, or less than or equal to 5%/cm. Combinations of these ranges are also possible (e.g., greater than or equal to 1%/cm and less than or equal to 5000%/cm, greater than or equal to 50%/cm and less than or equal to 600%/cm, or greater than or equal to 100%/cm and less than or equal to 600%/cm). Other ranges, both higher and lower than those described above, are also possible, as the disclosure is not so limited.

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) comprises a polymeric material having desirable mechanical properties. For example, in some embodiments, the polymeric material has a Young's elastic modulus in the first configuration (e.g., a water content less than the equilibrium water content state, such as the dehydrated state) (e.g., less than 5 w/w% water content) of greater than or equal to 100 MPa, greater than or equal to 250 MPa, greater than or equal to 500 MPa, greater than or equal to 600 MPa, greater than or equal to 750 MPa, greater than or equal to 800 MPa, greater than or equal to 900 MPa, greater than or equal to 1000 MPa, greater than or equal to 1250 MPa, greater than or equal to 1500 MPa, greater than or equal to 1750 MPa, greater than or equal to 2000 MPa, greater than or equal to 2500 MPa, greater than or equal to 3000 MPa, greater than or equal to 3500 MPa, or greater than or equal to 4000 MPa. In some embodiments, the polymeric material has a Young's elastic modulus in the first configuration (e.g., a water content less than the equilibrium water content state, such as the dehydrated state) (e.g., less than 5 w/w% water content) of less than or equal to 5000 MPa, less than or equal to 4000 MPa, less than or equal to 3500 MPa, less than or equal to 3000 MPa, less than or equal to 2500 MPa, less than or equal to 2000 MPa, less than or equal to 1750 MPa, less than or equal to 1500 MPa, less than or equal to 1250 MPa, less than or equal to 1000 MPa, less than or equal to 900 MPa, less than or equal to 800 MPa, less than or equal to 750 MPa, less than or equal to 600 MPa, less than or equal to 500 MPa, or less than or equal to 250 MPa. Combinations of the above-referenced ranges are also possible (e.g., greater than or equal to 100 MPa and less than or equal to 5000 MPa). Other ranges are also possible.

In some embodiments, the polymeric material has a Young's elastic modulus at an equilibrium water content state of less than or equal to 300 MPa, less than or equal to 250 MPa, less than or equal to 200 MPa, less than or equal to 150 MPa, less than or equal to 100 MPa, less than or equal to 75 MPa, less than or equal to 50 MPa, less than or equal to 25 MPa, less than or equal to 20 MPa, or less than or equal to 10 MPa. In some embodiments, the polymeric material has a Young's elastic modulus at an equilibrium water content state of greater than or equal to 5 MPa, greater than or equal to 10 MPa, greater than or equal to 20 MPa, greater than or equal to 25 MPa, greater than or equal to 50 MPa, greater than or equal to 75 MPa, greater than or equal to 100 MPa, greater than or equal to 150 MPa, greater than or equal to 200 MPa, or greater than or equal to 250 MPa. Combinations of the above-referenced ranges are also possible (e.g., less than or equal to 300 MPa and greater than or equal to 5 MPa). Other ranges are also possible.

As discussed above, the Young's elastic modulus (in the first state or at equilibrium water content) of a polymeric tube may vary. Where a polymeric tube has multiple portions (e.g., a first portion and a second portion), it should, of course be understood that the Young's elastic modulus of the polymeric tube (or within any portion thereof) may vary within any of the ranges identified in the foregoing paragraph, and that variation within other ranges is also possible. It should further be understood that where a polymeric tube comprises a plurality of portions having different physical properties, the average Young's elastic modulus of each portion may independently fall within one of the foregoing ranges.

A polymeric tube may have any of a variety of suitable spatial gradients of Young's elastic modulus. In some embodiments, the Young's elastic modulus has a gradient of greater than or equal to 0.1 MPa/cm, greater than or equal to 0.5 MPa/cm, greater than or equal to 1 MPa/cm, greater than or equal to 5 MPa/cm, greater than or equal to 10 MPa/cm, greater than or equal to 15 MPa/cm, greater than or equal to 20 MPa/cm, greater than or equal to 25 MPa/cm, greater than or equal to 30 MPa/cm, greater than or equal to 35 MPa/cm, greater than or equal to 40 MPa/cm, greater than or equal to 45 MPa/cm, greater than or equal to 50 MPa/cm, greater than or equal to 100 MPa/cm, greater than or equal to 200 MPa/cm, greater than or equal to 300 MPa/cm, or greater than or equal to 400 MPa/cm. In some embodiments, the Young's elastic modulus has a gradient of less than or equal to 500 MPa/cm, less than or equal to 400 MPa/cm, less than or equal to 300 MPa/cm, less than or equal to 200 MPa/cm, less than or equal to 100 MPa/cm, less than or equal to 50 MPa/cm, less than or equal to 45 MPa/cm, less than or equal to 40 MPa/cm, less than or equal to 35 MPa/cm, less than or equal to 30 MPa/cm, less than or equal to 25 MPa/cm, less than or equal to 20 MPa/cm, less than or equal to 15 MPa/cm, less than or equal to 10 MPa/cm, less than or equal to 5 MPa/cm, less than or equal to 1 MPa/cm, or less than or equal to 0.5 MPa/cm. Combinations of these ranges are also possible (e.g., greater than or equal to 0.1 MPa/cm and less than or equal to 500 MPa/cm, greater than or equal to 5 MPa/cm and less than or equal to 30 MPa/cm, or greater than or equal to 5 MPa/cm and less than or equal to 25 MPa/cm). Other ranges, both higher and lower than those described above, are also possible, as the disclosure is not so limited.

The Young's elastic modulus within the polymeric tube may vary within any of a variety of suitable ranges. One way to understand the extent of spatial variation is to consider the ratio of the overall maximum Young's elastic modulus of the polymeric material to the overall minimum Young's elastic modulus of the polymeric material. According to some embodiments, the ratio of the maximum to the minimum Young's elastic modulus of the polymeric tube (e.g., in the first state or at equilibrium water content) of the polymeric tube is greater than or equal to 1:1, greater than or equal to 5:1, greater than or equal to 10:1, greater than or equal to 15:1, greater than or equal to 20:1, greater than or equal to 25:1, greater than or equal to 30:1, greater than or equal to 35:1, greater than or equal to 40:1, greater than or equal to 45:1, greater than or equal to 50:1, or greater than or equal to 55:1. In some embodiments, the ratio of the maximum to the minimum Young's elastic modulus of the polymeric tube (e.g., in the first state or at equilibrium water content) of the polymeric tube is less than or equal to 60:1, less than or equal to 55:1, less than or equal to 50:1, less than or equal to 45:1, less than or equal to 40:1, less than or equal to 35:1, less than or equal to 30:1, less than or equal to 25:1, less than or equal to 20:1, less than or equal to 15:1, less than or equal to 10:1, or less than or equal to 5:1. Combinations of these ranges are also possible (e.g., greater than or equal to 1:1 and less than or equal to 60:1, or greater than or equal to 5:1 and less than or equal to 50:1). Other ranges, both higher and lower than those described above, are also possible, as the disclosure is not so limited.

While the foregoing ranges discuss Young's Elastic modulus, it should of course be understood that gradients in a variety of other mechanical properties, such as shear modulus, bulk modulus, Poisson ratio, toughness, ultimate tensile strength, yield strength, and/or, for anisotropic polymers, stiffness and/or compliance tensor coefficients, can also be established, depending on the embodiment.

The density of the polymeric tube may vary over any of a variety of suitable ranges. For example, In some embodiments, a polymeric tube has a density of greater than or equal to 1.00, greater than or equal to 1.005, greater than or equal to 1.01, greater than or equal to 1.02, greater than or equal to 1.05, greater than or equal to 1.10, greater than or equal to 1.15, greater than or equal to 1.20, greater than or equal to 1.25, greater than or equal to 1.30, or greater than or equal to 1.35. In some embodiments, a polymeric tube has a density of less than or equal to 1.40, less than or equal to 1.35, less than or equal to 1.30, less than or equal to 1.25, less than or equal to 1.20, less than or equal to 1.15, less than or equal to 1.10, less than or equal to 1.05, less than or equal to 1.02, less than or equal to 1.01, or less. Combinations of these ranges are also possible (e.g., greater than or equal to 1.00 and less than or equal to 1.40, greater than or equal to 1.005 and less than or equal to 1.30, or greater than or equal to 1.10 and less than or equal to 1.30). Other ranges, both higher and lower than those described above, are also possible, as the disclosure is not so limited.

In some embodiments, the plurality of pores (e.g., the pores of polymeric tube 12 of FIG. 5B, polymeric tube 14 of FIG. 5C) or of a first water soluble material, optionally having a second water soluble polymer positioned within at least a portion of said pores) have a particular mean pore size. In some embodiments, the mean pore size of the plurality of pores is less than or equal to 500 nm, less than or equal to 450 nm, less than or equal to 400 nm, less than or equal to 350 nm, less than or equal to 300 nm, less than or equal to 250 nm, less than or equal to 200 nm, less than or equal to 150 nm, less than or equal to 100 nm, less than or equal to 75 nm, less than or equal to 50 nm, less than or equal to 25 nm, less than or equal to 20 nm, or less than or equal to 15 nm. In some embodiments, the plurality of pores have a mean pore size of greater than or equal to 10 nm, greater than or equal to 15 nm, greater than or equal to 20 nm, greater than or equal to 25 nm, greater than or equal to 50 nm, greater than or equal to 75 nm, greater than or equal to 100 nm, greater than or equal to 150 nm, greater than or equal to 200 nm, greater than or equal to 250 nm, greater than or equal to 300 nm, greater than or equal to 350 nm, greater than or equal to 400 nm, or greater than or equal to 450 nm. Combinations of the above referenced ranges are also possible (e.g., less than or equal to 500 nm and greater than or equal to 10 nm). Other ranges are also possible. Mean pore size, as described herein, may be determined by mercury intrusion porosimetry of the material in a first configuration (e.g., a water content less than the equilibrium water content state, such as the dehydrated state).

In some embodiments, at least a portion of the plurality of pores may be characterized as nanopores, e.g., pores having an average cross-sectional dimension of less than 1 micron. In some embodiments, at least a portion of the plurality of pores may be characterized as micropores, e.g., pores having an average cross-sectional dimension of less than 1 mm and greater than or equal to 1 micron. In some embodiments, at least 50% (e.g., at least 60%, at least 70%, at least 80%, at least 90%, at least 95%, at least 98%, at least 99%, at least 99.9%) of the plurality of pores have a diameter that is less than or equal to 1 micron, less than or equal to 800 nm, less than or equal to 600 nm, less than or equal to 500 nm, less than or equal to 450 nm, less than or equal to 400 nm, less than or equal to 350 nm, less than or equal to 300 nm, less than or equal to 250 nm, less than or equal to 200 nm, less than or equal to 150 nm, less than or equal to 100 nm, less than or equal to 75 nm, less than or equal to 50 nm, less than or equal to 25 nm, less than or equal to 20 nm, or less than or equal to 15 nm. In some cases, at least 50% of the plurality of pores have a diameter that is greater than or equal to 10 nm, greater than or equal to 15 nm, greater than or equal to 20 nm, greater than or equal to 25 nm, greater than or equal to 50 nm, greater than or equal to 75 nm, greater than or equal to 100 nm, greater than or equal to 150 nm, greater than or equal to 200 nm, greater than or equal to 250 nm, greater than or equal to 300 nm, greater than or equal to 350 nm, greater than or equal to 400 nm, greater than or equal to 450 nm, greater than or equal to 500 nm, greater than or equal to 600 nm, or greater than or equal to 800 nm. Combinations of the above referenced ranges are also possible (e.g., less than or equal to 1000 nm and greater than or equal to 10 nm). Other ranges are also possible.

The compositions, devices and polymeric tubes (e.g., polymeric tube 10 of FIG. 5A, polymeric tube 12 of FIG. 5B, polymeric tube 14 of FIG. 5C) described herein may have a particular porosity e.g., in a first configuration (e.g., a water content less than the equilibrium water content state, such as the dehydrated state). In some embodiments, the polymeric tube (or polymeric material) has a porosity of greater than or equal to 5%, greater than or equal to 10%, greater than or equal to 15%, greater than or equal to 20%, greater than or equal to 25%, greater than or equal to 30%, greater than or equal to 35%, greater than or equal to 40%, or greater than or equal to 45% in a first configuration (e.g., a water content less than the equilibrium water content state, such as the dehydrated state). In some embodiments, the polymeric tube (or polymeric material) has a porosity of less than or equal to 50%, less than or equal to 45%, less than or equal to 40%, less than or equal to 35%, less than or equal to 30%, less than or equal to 25%, less than or equal to 20%, less than or equal to 15%, or less than or equal to 10% in a first configuration (e.g., a water content less than the equilibrium water content state, such as the dehydrated state). Combinations of the above-referenced ranges are also possible (e.g., greater than or equal to 5% and less than or equal to 50% in a first configuration (e.g., a water content less than the equilibrium water content state, such as the dehydrated state)). Other ranges are also possible.

It should, of course, be understood that the above-referenced porosities and pore-sizes may be varied spatially within a polymeric tube, according to some embodiments. For example, in some embodiments, the polymeric tube comprises a first portion having a first porosity or pore size and a second portion having a second porosity or pore size, wherein the second porosity or pore size differs from the first porosity or pore size. Average values of porosity or pore size for different portions of the polymeric tube may each, individually, fall within one or more of the foregoing ranges. Likewise, the average value of porosity or pore size for the entire polymeric tube may, in some embodiments, fall within one of the foregoing ranges.

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) is hydrophilic. The term “hydrophilic” as used herein is given its ordinary meaning in the art and refers to a material surface having a water contact angle as determined by goniometry of less than 90 degrees. In some embodiments, the polymeric material (or a surface thereof) (e.g., of the polymeric tube) has a water contact angle of less than or equal to 45 degrees, less than or equal to 40 degrees, less than or equal to 35 degrees, less than or equal to 30 degrees, less than or equal to 25 degrees, less than or equal to 20 degrees, less than or equal to 15 degrees, less than or equal to 10 degrees, less than or equal to 5 degrees, or less than or equal to 2 degrees at an equilibrium water content state. In some embodiments, the polymeric material (or a surface thereof) has a water contact angle of greater than or equal to 1 degree, greater than or equal to 2 degrees, greater than or equal to 5 degrees, greater than or equal to 10 degrees, greater than or equal to 15 degrees, greater than or equal to 20 degrees, greater than or equal to 25 degrees, greater than or equal to 30 degrees, greater than or equal to 35 degrees, or greater than or equal to 40 degrees at an equilibrium water content state. Combinations of the above-referenced ranges are also possible (e.g., greater than or equal to 1 degree and less than or equal to 45 degrees). Other ranges are also possible.

It should, of course, be understood that the above-referenced water contact angle may be varied spatially within a polymeric tube, according to some embodiments. For example, in some embodiments, the polymeric tube comprises a first portion having a first water contact angle and a second portion having a second water contact angle, wherein the second water contact angle differs from the first water contact angle. Average values of water contact angle for different portions of the polymeric tube may each, independently, fall within one or more of the foregoing ranges. Likewise, the average value of water contact angle for the entire polymeric tube may, in some embodiments, fall within one of the foregoing ranges.

In some embodiments, the polymeric tube (e.g., polymeric tube 10 of FIG. 5A, polymeric tube 12 of FIG. 5B, polymeric tube 14 of FIG. 5C) is substantially lubricious at an equilibrium water content state. For example, in some embodiments, the polymeric tube (or polymeric material of the polymeric tube) has a surface roughness of less than or equal to 1000 nm (Ra) at an equilibrium water content state. In some embodiments, the polymeric tube (or polymeric material of the polymeric tube) has a surface roughness (Ra) of less than or equal to 500 nm, less than or equal to 400 nm, less than or equal to 300 nm, less than or equal to 250 nm, less than or equal to 200 nm, less than or equal to 150 nm, less than or equal to 100 nm, less than or equal to 50 nm, less than or equal to 25 nm, less than or equal to 10 nm, or less than or equal to 5 nm at an equilibrium water content state. In some embodiments, the polymeric tube (or polymeric material of the polymeric tube) has a surface roughness (Ra) of greater than or equal to 5 nm at an equilibrium water content state, greater than or equal to 10 nm, greater than or equal to 25 nm, greater than or equal to 50 nm, greater than or equal to 100 nm, greater than or equal to 150 nm, greater than or equal to 200 nm, greater than or equal to 250 nm, greater than or equal to 300 nm, greater than or equal to 400 nm, or greater than or equal to 500 nm at an equilibrium water content state. Combinations of the above-referenced ranges are also possible (e.g., greater than or equal to 5 nm and less than or equal to 1000 nm). Other ranges are also possible.

It should, of course, be understood that the above-referenced surface roughness may be varied spatially within a polymeric tube, according to some embodiments. For example, in some embodiments, the polymeric tube comprises a first portion having a first surface roughness and a second portion having a second surface roughness, wherein the second surface roughness differs from the first surface roughness. Average values of surface roughness for different portions of the polymeric tube may each, independently, fall within one or more of the foregoing ranges. Likewise, the average value of surface roughness for the entire polymeric tube may, in some embodiments, fall within one of the foregoing ranges.

In some embodiments, the polymeric tube (e.g., polymeric tube 10 of FIG. 5A, polymeric tube 12 of FIG. 5B, polymeric tube 14 of FIG. 5C) has a surface having a coefficient of friction of less than or equal to 0.10 at an equilibrium water content state. For example, the coefficient of friction of a surface of the polymeric tube (or polymeric material of the polymeric tube) is less than or equal to 0.1, less than or equal to 0.09, less than or equal to 0.08, less than or equal to 0.07, less than or equal to 0.06, less than or equal to 0.05, less than or equal to 0.04, less than or equal to 0.03, or less than or equal to 0.02. In some embodiments, the coefficient of friction of the surface of the polymeric tube (or polymeric material of the polymeric tube) is greater than or equal to 0.01, greater than or equal to 0.02, greater than or equal to 0.03, greater than or equal to 0.04, greater than or equal to 0.05, greater than or equal to 0.06, greater than or equal to 0.07, greater than or equal to 0.08, or greater than or equal to 0.09. Combinations of the above-referenced ranges are also possible (e.g., less than or equal to 0.1 and greater than or equal to 0.01). Other ranges are also possible.

It should, of course, be understood that the above-referenced coefficient of friction may be varied spatially within a polymeric tube, according to some embodiments. For example, in some embodiments, the polymeric tube comprises a first portion having a first coefficient of friction and a second portion having a second coefficient of friction, wherein the second coefficient of friction differs from the first coefficient of friction. Average values of coefficient of friction for different portions of the polymeric tube may each, independently, fall within one or more of the foregoing ranges. Likewise, the average value of coefficient of friction for the entire polymeric tube may, in some embodiments, fall within one of the foregoing ranges.

Advantageously, the compositions, polymeric tubes, articles, and devices described herein may have low sorption of substances such as therapeutic agents (and/or e.g., proteins) in the presence of a dynamic fluid comprising such substances. Such devices, articles, polymeric tubes, and compositions may be useful for use in subjects where, for example, the presence of the composition, polymeric tube, article, or device should not substantially decrease the availability and/or concentration of therapeutic agents delivered to the subject (e.g., via the polymeric tube). In some embodiments, administration of therapeutic agents via a fluid flowed within the polymeric tubes described herein do not substantially reduce the concentration of the therapeutic agent within the fluid. In some cases, the polymeric tube may not absorb and/or adsorb the therapeutic agent, e.g., during flow or use.

In some embodiments, less than or equal to 0.5 w/w% sorption of a therapeutic agent to the surface and/or bulk of the first water-soluble polymer occurs as determined at equilibrium water content after exposing the polymer to the therapeutic agent and flushing with 5 times the volume of the polymeric tube with an aqueous solution, such as water or normal saline. In some embodiments, less than or equal to 0.5 w/w%, less than or equal to 0.4 w/w%, less than or equal to 0.3 w/w%, less than or equal to 0.2 w/w%, or less than or equal to 0.1 w/w% sorption of the therapeutic agent to the surface and/or bulk of the first water-soluble polymer occurs. In some embodiments, greater than or equal to 0.05 w/w%, greater than or equal to 0.1 w/w%, greater than or equal to 0.2 w/w%, greater than or equal to 0.3 w/w%, or greater than or equal to 0.4 w/w% sorption of the therapeutic agent to the surface and/or bulk of the first water-soluble polymer occurs. Combinations of the above-referenced ranges are also possible (e.g., less than or equal to 0.5 w/w% and greater than or equal to 0.05 w/w%). Other ranges are also possible.

It should, of course, be understood that the above-referenced sorption of a therapeutic agent may be varied spatially within a polymeric tube, according to some embodiments. For example, in some embodiments, the polymeric tube comprises a first portion having a first sorption of a therapeutic agent and a second portion having a second sorption of a therapeutic agent, wherein the second sorption of a therapeutic agent differs from the first sorption of a therapeutic agent. Average values of sorption of a therapeutic agent for different portions of the polymeric tube may each, independently, fall within one or more of the foregoing ranges. Likewise, the average value of sorption of a therapeutic agent for the entire polymeric tube may, in some embodiments, fall within one of the foregoing ranges.

Equilibrium water content state, as used herein, refers the steady state of a polymeric tube (or composition) which does not gain (e.g., absorb) or lose bulk water content as determined when submerged in water at 25° C. without externally applied mechanical stresses. Those skilled in the art would understand that steady state (or equilibrium water content state) shall be understood to not require absolute conformance to a strict thermodynamic definition of such term, but, rather, shall be understood to indicate conformance to the thermodynamic definition of such term to the extent possible for the subject matter so characterized as would be understood by one skilled in the art most closely related to such subject matter (e.g., accounting for factors such as passive diffusion and/or Brownian motion).

In some embodiments, the equilibrium water content state of the polymeric tube (or polymeric material) is greater than or equal to 10 w/w%, greater than or equal to 20 w/w%, greater than or equal to 25 w/w%, greater than or equal to 30 w/w%, greater than or equal to 35 w/w%, greater than or equal to 40 w/w%, greater than or equal to 45 w/w%, greater than or equal to 50 w/w%, greater than or equal to 55 w/w%, greater than or equal to 60 w/w%, greater than or equal to 65 w/w%, or greater than or equal to 70 w/w%. In some embodiments, the equilibrium water content state of the polymeric tube (or polymeric material) is less than or equal to 80 w/w%, less than or equal to 75 w/w%, less than or equal to 70 w/w%, less than or equal to 65 w/w%, less than or equal to 60 w/w%, less than or equal to 55 w/w%, less than or equal to 50 w/w%, less than or equal to 45 w/w%, less than or equal to 40 w/w%, less than or equal to 35 w/w%, less than or equal to 30 w/w%, less than or equal to 25 w/w%, or less than or equal to 20 w/w%. Combinations of these ranges are also possible (e.g., greater than or equal to 10 w/w% and less than or equal to 80 w/w%). Other ranges are also possible.

Advantageously, the polymeric tubes and compositions described herein may have desirable swelling characteristics (e.g., in water, in saline, in a fluidic environment of a subject).

It should, of course, be understood that the above-referenced equilibrium water content state may be varied spatially within a polymeric tube, according to some embodiments. For example, in some embodiments, the polymeric tube comprises a first portion having a first equilibrium water content state and a second portion having a second equilibrium water content state, wherein the second equilibrium water content state differs from the first equilibrium water content state. Average values of equilibrium water content state for different portions of the polymeric tube may each, independently, fall within one or more of the foregoing ranges. Likewise, the average value of equilibrium water content state for the entire polymeric tube may, in some embodiments, fall within one of the foregoing ranges.

In some embodiments, the polymeric tubes (or polymeric materials) described herein have a first configuration (e.g., a water content less than the equilibrium water content state, such as the dehydrated state) with a water content of less than or equal to 40 w/w%, less than or equal to 30 w/w%, less than or equal to 20 w/w%, less than or equal to 10 w/w%, less than or equal to 5 w/w%, less than or equal to 4 w/w%, less than or equal to 3 w/w%, less than or equal to 2 w/w%, less than or equal to 1 w/w%, less than or equal to 0.8 w/w%, less than or equal to 0.6 w/w%, less than or equal to 0.4 w/w%, or less than or equal to 0.2 w/w%. In some embodiments, the polymeric tubes (or polymeric materials) described herein have a first configuration (e.g., a water content less than the equilibrium water content state, such as the dehydrated state) with a water content of greater than or equal to 0.1 w/w%, greater than or equal to 0.2 w/w%, greater than or equal to 0.4 w/w%, greater than or equal to 0.6 w/w%, greater than or equal to 0.8 w/w%, greater than or equal to 1 w/w%, greater than or equal to 2 w/w%, greater than or equal to 3 w/w%, greater than or equal to 4 w/w%, greater than or equal to 5 w/w%, greater than or equal to 6 w/w%, greater than or equal to 7 w/w%, greater than or equal to 8 w/w%, greater than or equal to 9 w/w%, greater than or equal to 10 w/w%, greater than or equal to 15 w/w, greater than or equal to 20 w/w%, greater than or equal to 25 w/w%, greater than or equal to 30 w/w%, or greater than or equal to 35 w/w%. Combinations of the above-referenced ranges are also possible (e.g., greater than or equal to 0.1 w/w% and less than 5 w/w%, greater than or equal to 2 w/w% and less than or equal to 10 w/w%, greater than or equal to 2 w/w% and less than or equal to 40 w/w%, or greater than or equal to 6 w/w% and less than or equal to 40 w/w%). Other ranges are also possible. The dehydrated state, as described herein, generally refers to the steady state determined under ambient conditions in which the polymeric tube (or polymeric material) has no appreciable decrease in water content of less than 5 w/w% over 24 hours. In some embodiments, the polymeric tubes described herein may comprise a coating or unbound porogen, such as a humectant coating, as described in more detail below.

It should, of course, be understood that the above-referenced first configuration water content may be varied spatially within a polymeric tube, according to some embodiments. For example, in some embodiments, the polymeric tube comprises a first portion having a first first configuration water content and a second portion having a second first configuration water content, wherein the second first configuration water content differs from the first first configuration water content. Average values of first configuration water content for different portions of the polymeric tube may each, independently, fall within one or more of the foregoing ranges. Likewise, the average value of first configuration water content for the entire polymeric tube may, in some embodiments, fall within one of the foregoing ranges.

In some embodiments, the polymeric tubes (or polymeric materials) described herein have a first configuration (e.g., a water content less than the equilibrium water content state, such as the dehydrated state). In some embodiments, the polymeric tubes (or polymeric materials) described herein swell from a first configuration (e.g., a water content less than the equilibrium water content state, such as the dehydrated state) to a second configuration (e.g., the equilibrium water content state) in less than or equal to 60 minutes (e.g., less than or equal to 10 minutes, less than or equal to 5 minutes, less than or equal to 1 minute, or less than or equal to 10 seconds). In some embodiments, the polymeric tubes (or polymeric materials) described herein swell from a first configuration (e.g., a water content less than the equilibrium water content state, such as the dehydrated state) to a second configuration (e.g., the equilibrium water content state) at 25° C.

In some embodiments, the polymeric tubes (or polymeric materials) described herein swell in an amount greater than or equal to 2 w/w%, greater than or equal to 3 w/w%, greater than or equal to 4 w/w%, greater than or equal to 5 w/w%, greater than or equal to 10 w/w%, greater than or equal to 15 w/w%, greater than or equal to 20 w/w%, greater than or equal to 25 w/w%, greater than or equal to 30 w/w%, greater than or equal to 35 w/w%, greater than or equal to 40 w/w%, or greater than or equal to 45 w/w%, for example, from a first configuration (e.g., a water content less than the equilibrium water content state, such as the dehydrated state) to a second configuration (e.g., the equilibrium water content state). In some embodiments, the polymeric tubes (or polymeric materials) described herein swell in an amount less than or equal to 50 w/w%, less than or equal to 45 w/w%, less than or equal to 40 w/w%, less than or equal to 35 w/w%, less than or equal to 30 w/w%, less than or equal to 25 w/w%, less than or equal to 20 w/w%, less than or equal to 15 w/w%, less than or equal to 10 w/w%, less than or equal to 5 w/w%, less than or equal to 4 w/w%, or less than or equal to 3 w/w%, for example, from a first configuration (e.g., a water content less than the equilibrium water content state, such as the dehydrated state) to a second configuration (e.g., the equilibrium water content state). Combinations of these ranges are also possible (e.g., greater than or equal to 5 w/w% and less than or equal to 40 w/w%).

Advantageously, the polymeric tubes and compositions described herein may be configured for rapid swelling in the presence of an aqueous solution, such as water and/or saline. In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5C) or polymeric material) is configured to swell in an amount greater than or equal to 2 w/w%, greater than or equal to 5 w/w%, greater than or equal to 10 w/w%, greater than or equal to 15 w/w%, greater than or equal to 20 w/w%, greater than or equal to 25 w/w%, greater than or equal to 30 w/w%, greater than or equal to 35 w/w%, greater than or equal to 40 w/w%, or greater than or equal to 45 w/w%, for example, from a first configuration (e.g., a water content less than the equilibrium water content state, such as the dehydrated state) to a second configuration (e.g., equilibrium water content state), e.g., at 25° C., e.g., in a particular amount of time (e.g., less than or equal to 60 minutes, less than or equal to 10 minutes, less than or equal to 5 minutes, less than or equal to 1 minute, or less than or equal to 10 seconds), as described in more detail below. In some embodiments, the polymeric tube (or body portion) is configured to swell in an amount less than or equal to 50 w/w%, less than or equal to 45 w/w%, less than or equal to 40 w/w%, less than or equal to 35 w/w%, less than or equal to 30 w/w%, less than or equal to 25 w/w%, less than or equal to 20 w/w%, less than or equal to 15 w/w%, or less than or equal to 10 w/w%, for example, from a first configuration (e.g., a water content less than the equilibrium water content state, such as the dehydrated state) to a second configuration (e.g., an equilibrium water content state), e.g., at 25° C., e.g., in a particular amount of time (e.g., less than or equal to 60 minutes, less than or equal to 10 minutes, less than or equal to 5 minutes, less than or equal to 1 minute, or less than or equal to 10 seconds) as described in more detail below. Combinations of the above-referenced ranges are also possible (e.g., greater than or equal to 5 w/w% and less than or equal to 50 w/w%). Other ranges are also possible.

It should, of course, be understood that the above-referenced swelling e.g., from the first configuration to the second configuration may be varied spatially within a polymeric tube, according to some embodiments. For example, in some embodiments, the polymeric tube comprises a first portion having a first swelling and a second portion having a second swelling, wherein the second swelling differs from the first swelling. Average values of swelling for different portions of the polymeric tube may each, independently, fall within one or more of the foregoing ranges. Likewise, the average value of swelling for the entire polymeric tube may, in some embodiments, fall within one of the foregoing ranges.

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) is configured to swell in an amount greater than or equal to 2 w/w%, greater than or equal to 5 w/w%, for example, from a first configuration (e.g., a water content less than the equilibrium water content state, such as the dehydrated state) to a second configuration (e.g., an equilibrium water content state), in less than or equal to 60 minutes, less than or equal to 50 minutes, less than or equal to 40 minutes, less than or equal to 30 minutes, less than or equal to 20 minutes, less than or equal to 10 minutes, less than or equal to 5 minutes, less than or equal to 2 minutes, less than or equal to 1 minute, less than or equal to 30 seconds, or less than or equal to 10 seconds at 25° C. In some embodiments, the polymeric tube (or polymeric material) is configured to swell in an amount greater than or equal to 5 w/w%, for example, from a first configuration (e.g., a water content less than the equilibrium water content state, such as the dehydrated state) to a second configuration (e.g., an equilibrium water content state), in greater than or equal to 5 seconds, greater than or equal to 15 seconds, greater than or equal to 1 minute, greater than or equal to 2 minutes, greater than or equal to 5 minutes, greater than or equal to 10 minutes, greater than or equal to 20 minutes, greater than or equal to 30 minutes, greater than or equal to 40 minutes, or greater than or equal to 50 minutes at 25° C. Combinations of the above-referenced ranges are also possible (e.g., less than or equal to 60 minutes and greater than or equal to 1 minute). Other ranges are also possible.

In an exemplary embodiment, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) is configured to swell to an equilibrium water content state (e.g., greater than or equal to 5 w/w% or greater than or equal to 20 w/w% and less than or equal to 80 w/w%) in less than or equal to 60 minutes (e.g., less than or equal to 10 minutes, less than or equal to 5 minutes, less than or equal to 1 minute, or less than or equal to 10 seconds) from a first configuration (e.g., a water content less than the equilibrium water content state, such as the dehydrated state) (e.g., less than 5 w/w% or greater than or equal to 2 w/w% and less than or equal to 40 w/w%) in water. In some embodiments, the polymeric tube (or polymeric material) is configured to swell to an equilibrium water content (e.g., greater than or equal to 5 w/w% or greater than or equal to 20 w/w% and less than or equal to 80 w/w%) in less than or equal to 60 minutes (e.g., less than or equal to 10 minutes, less than or equal to 5 minutes, less than or equal to 1 minute, or less than or equal to 10 seconds) from, for example, a first configuration (e.g., a water content less than the equilibrium water content state, such as the dehydrated state) (e.g., less than 5 w/w%) in standard normal saline. In another exemplary embodiment, the polymeric tube (or polymeric material) is configured to swell to an equilibrium water content (e.g., greater than or equal to 5 w/w% or greater than or equal to 20 w/w% and less than or equal to 80 w/w%) in less than or equal to 60 minutes (e.g., less than or equal to 10 minutes, less than or equal to 5 minutes, less than or equal to 1 minute, or less than or equal to 10 seconds) from, for example, a first configuration (e.g., a water content less than the equilibrium water content state, such as the dehydrated state) (e.g., less than 5 w/w%) in normal saline.

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) has a particular length in the first configuration (e.g., a water content less than the equilibrium water content state, such as the dehydrated state). In some embodiments, the polymeric tube (or polymeric material) has an increase in overall length in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to its length in a first configuration (e.g., a water content less than the equilibrium water content state, such as the dehydrated state). In some cases, the polymeric tube (or polymeric material) has an increase in overall length in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to its length in the first configuration (e.g., a water content less than the equilibrium water content state, such as the dehydrated state). Combinations of the above-referenced ranges are also possible (e.g., greater than or equal to 0.1% and less than or equal to 20%). Other ranges are also possible.

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) has a particular outer maximum transverse dimension, such as an outer diameter of a cylindrical tube, an oval tube, an oblong tube, or square tube. In embodiments where the device comprises multiple lumens, the outer diameter refers to the outer maximum transverse dimension of one or more of the lumens. For example, in some embodiments only one lumen may have the recited outer diameter. In other embodiments, each and every lumen may independently have the recited outer diameter. In some embodiments, the polymeric tube (or polymeric material) has an increase in an outer maximum transverse dimension (e.g., outer diameter) in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to the maximum transverse dimension (e.g., outer diameter) in a first configuration (e.g., a water content less than the equilibrium water content state, such as the dehydrated state). In some cases, the polymeric tube (or polymeric material) has an increase in the maximum transverse dimension (e.g., outer diameter) in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to the maximum transverse dimension (e.g., outer diameter) in a first configuration (e.g., a water content less than the equilibrium water content state, such as the dehydrated state). Combinations of the above-referenced ranges are also possible (e.g., greater than or equal to 0.1% and less than or equal to 20%, greater than or equal to 0.1% and less than or equal to 10%). Other ranges are also possible.

In some embodiments, the polymeric tube (or body portion) has a particular inner diameter (e.g., in an embodiment in which the polymeric tube comprises a lumen), which is the maximum inner transverse dimension, such as the inner diameter of a cylindrical tube or square tube (or other non-circular polymeric tube or body portion). In embodiments where the polymeric tube (or body portion) comprises multiple lumens, the inner diameter refers to the maximum inner transverse dimension (i.e., the maximum inner transverse dimension of the largest lumen). In some embodiments, the polymeric tube (or body portion) has an increase in the inner diameter in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to the inner diameter in a first configuration (e.g., a water content less than the equilibrium water content state, such as the dehydrated state). In some cases, the polymeric tube (or body portion) has an increase in the inner diameter in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to the inner diameter in a first configuration (e.g., a water content less than the equilibrium water content state, such as the dehydrated state). Combinations of the above-referenced ranges are also possible (e.g., greater than or equal to 0.1% and less than or equal to 20%). Other ranges are also possible.

In some embodiments, the polymeric tube (or body portion) has a larger percentage increase in the overall length than an increase in inner diameter and/or outer diameter when the polymeric tube (or polymeric material) swells from a first configuration (e.g., a water content less than the equilibrium water content state, such as the dehydrated state) to a second configuration (e.g., the equilibrium water content state). For example, in some embodiments, the overall length may increase by 1-20% (e.g., 5-15%) while the inner diameter and/or outer diameter increases by 0.1-19% (e.g., 1-10%).

In some embodiments, the ratio of the percentage increase in the overall length to the percentage increase in the inner diameter and/or outer diameter when the polymeric tube (or polymeric material) swells from a first configuration (e.g., a water content less than the equilibrium water content state, such as the dehydrated state) to a second configuration (e.g., the equilibrium water content state) is greater than or equal to 1.1, greater than or equal to 1.5, greater than or equal to 2, greater than or equal to 5, greater than or equal to 7, or greater than or equal to 10. In some embodiments, the ratio of the percentage increase in the overall length to the percentage increase in the inner diameter and/or outer diameter when the polymeric tube (or polymeric material) swells from a first configuration (e.g., a water content less than the equilibrium water content state, such as the dehydrated state) to a second configuration (e.g., the equilibrium water content state) is less than or equal to 20, less than or equal to 15, less than or equal to 10, less than or equal to 5, or less than or equal to 2. Combinations of these ranges are also possible (e.g., 1.1-20).

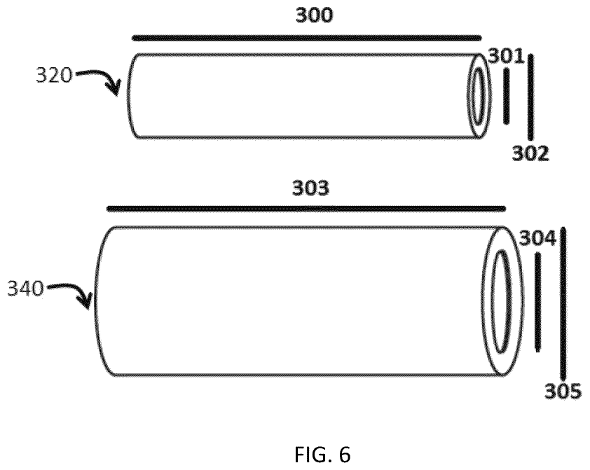

In some embodiments, the polymeric tube (or body portion) has a larger percentage increase in the inner diameter and/or outer diameter than in overall length when the polymeric tube (or polymeric material) swells from a first configuration (e.g., a water content less than the equilibrium water content state, such as the dehydrated state). As a non-limiting example, in FIG. 6, polymeric tube 320 swelled from a first configuration (e.g., a water content less than the equilibrium water content state, such as the dehydrated state) to a second configuration (e.g., the equilibrium water content state)—polymeric tube 340. In accordance with some embodiments, in FIG. 6, outer diameter 302 and inner diameter 301 of polymeric tube 320 increased to outer diameter 305 and inner diameter 304 in polymeric tube 340, respectively, while overall length 300 increased to overall length 303. In accordance with some embodiments, in FIG. 6, inner diameter 301 and outer diameter 302 increased by a larger percentage than the increase in overall length 300 when polymeric tube 320 swelled to the equilibrium water content state—polymeric tube 340. In some embodiments, the inner diameter and/or outer diameter may increase by 1-20% (e.g., 5-15%) while the overall length increases by 0.1-19% (e.g., 1-10%).

In some embodiments, the ratio of the percentage increase in the inner diameter and/or outer diameter to the percentage increase in the overall length when the polymeric tube (or polymeric material) swells from a first configuration (e.g., a water content less than the equilibrium water content state, such as the dehydrated state) to a second configuration (e.g., the equilibrium water content state) is greater than or equal to 1.1, greater than or equal to 1.5, greater than or equal to 2, greater than or equal to 5, greater than or equal to 7, or greater than or equal to 10. In some embodiments, the ratio of the percentage increase in the inner diameter and/or outer diameter to the percentage increase in the overall length when the polymeric tube (or polymeric material) swells from a first configuration (e.g., a water content less than the equilibrium water content state, such as the dehydrated state) to a second configuration (e.g., the equilibrium water content state) is less than or equal to 20, less than or equal to 10, less than or equal to 5,or less than or equal to 2. Combinations of these ranges are also possible (e.g., 1.1-20).

In some embodiments, the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) comprises an osmotic agent. For example, in some embodiments, an osmotic agent may be added (e.g., to the pre-polymer) during formation of the polymeric tube. In some embodiments, the osmotic agent is present in the polymeric material (e.g., after formation of the polymeric material) in an amount greater than or equal to 0.05 w/w%, greater than or equal to 0.1 w/w%, greater than or equal to 0.2 w/w%, greater than or equal to 0.4 w/w%, greater than or equal to 0.6 w/w%, greater than or equal to 0.8 w/w%, greater than or equal to 1 w/w%, greater than or equal to 1.2 w/w%, greater than or equal to 1.4 w/w%, greater than or equal to 1.6 w/w%, or greater than or equal to 1.8 w/w% versus the total polymeric tube weight in a first configuration (e.g., dehydrated state) and/or second configuration (e.g., equilibrium water content state). In some cases, the osmotic agent may be present in the polymeric material (e.g., after formation of the polymeric material) in an amount of less than or equal to 2 w/w%, less than or equal to 1.8 w/w%, less than or equal to 1.6 w/w%, less than or equal to 1.4 w/w%, less than or equal to 1.2 w/w%, less than or equal to 1 w/w%, less than or equal to 0.8 w/w%, less than or equal to 0.6 w/w%, less than or equal to 0.4 w/w%, less than or equal to 0.2 w/w%, or less than or equal to 0.01 w/w% versus the total polymeric tube weight in a first configuration (e.g., dehydrated state) and/or second configuration (e.g., equilibrium water content state). Combinations of the above-referenced ranges are also possible (e.g., greater than or equal to 0.05 w/w% and less than or equal to 2 w/w%). Other ranges are also possible.

Non-limiting examples of suitable osmotic agents include phosphates, borates, sodium chloride, citrates, ethylenediaminetetraacetates, sulfites, sulfates, hyposulfites, metal oxides, selenium dioxide, selenium trioxide, selenous acid, selenic acid, nitrates, silicates, and botanic acid.

In some embodiments, the composition (e.g., comprising or formed of a polymeric material) and/or the first water soluble polymer does not comprise covalent crosslinking, as described in more detail below. In other embodiments, however, the composition and/or the first water soluble polymer comprises physical crosslinking (e.g., interpenetrating network, chain entanglement, and/or one or more bonds such as covalent, ionic, and/or hydrogen bonding). In a particular set of embodiments, no covalent crosslinking agents are used to form the polymeric material, the first water soluble polymer of the polymeric material, and/or the second water soluble polymer.

The first water soluble polymer may be present in the polymeric tube (e.g., polymeric tube 10 of FIG. 5A, polymeric tube 12 of FIG. 5B, polymeric tube 14 of FIG. 5C) (or body portion (e.g., body portion 20 of FIGS. 5A-5B)) in any suitable amount. For example, in some embodiments, the first water soluble polymer is present in the polymeric tube and/or body portion in an amount of greater than or equal to 20 w/w%, greater than or equal to 25 w/w%, greater than or equal to 30 w/w%, greater than or equal to 35 w/w%, greater than or equal to 40 w/w%, greater than or equal to 45 w/w%, greater than or equal to 50 w/w%, greater than or equal to 55 w/w%, greater than or equal to 60 w/w%, greater than or equal to 65 w/w%, greater than or equal to 70 w/w%, greater than or equal to 75 w/w%, greater than or equal to 80 w/w%, greater than or equal to 85 w/w%, or greater than or equal to 90 w/w% at an equilibrium water content state. In some embodiments, the first water soluble polymer is present in the polymeric tube and/or body portion in an amount of less than or equal to 95 w/w%, less than or equal to 90 w/w%, less than or equal to 85 w/w%, less than or equal to 80 w/w%, less than or equal to 75 w/w%, less than or equal to 70 w/w%, less than or equal to 65 w/w%, less than or equal to 60 w/w%, less than or equal to 55 w/w%, less than or equal to 50 w/w%, less than or equal to 45 w/w%, less than or equal to 40 w/w%, less than or equal to 35 w/w%, less than or equal to 30 w/w%, or less than or equal to 25 w/w% at an equilibrium water content state. Combinations of the above-referenced ranges are also possible (e.g., greater than or equal to 20 w/w% and less than or equal to 95 w/w%). Other ranges are also possible.

In some embodiments, the first water soluble polymer comprises or is selected from the group consisting of poly(vinyl alcohol) (PVA), poly(acrylic acid) (PAA), polyethylene glycol (PEG), poly(vinyl pyrrolidone) (PVP), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In an exemplary set of embodiments, the first water soluble polymer is poly(vinyl alcohol).

In some embodiments, the polymeric material (e.g., the polymeric tube) comprises a mixture comprising the first water-soluble polymer and another (e.g., a third) water soluble polymer. In some embodiments, the third water soluble polymer comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. The first and other (e.g., third) water soluble polymers may have different chemical compositions.

In some embodiments, the polymeric material (e.g., the polymeric tube) comprises one or more coatings comprising a (e.g., a fourth) water soluble polymer. The coating may, for example, help to allow a portion of the polymeric material to remain flexible and/or reduce dehydration of at least a portion of the polymeric material prior to implantation. In some embodiments, the water soluble polymer comprises or is selected from the group consisting of poly(vinyl alcohol) (PVA), poly(acrylic acid) (PAA), polyethylene glycol (PEG), poly(vinyl pyrrolidone) (PVP), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations or copolymers thereof.

In some embodiments, a water soluble polymer of a coating (e.g., a fourth water-soluble polymer) is comparatively more hydrophobic than one or more other water soluble polymers of the polymeric material (e.g., the polymeric tube). In some embodiments, for example, the fourth water-soluble polymer comprises vinyl acetate. For example, in some embodiments, the fourth water-soluble polymer is copolymerized with a less water-soluble monomer. For example, in some embodiments, the fourth water-soluble polymer is or comprises poly(vinyl alcohol-co-vinyl acetate). In another exemplary set of embodiments, the fourth water soluble polymer is or comprises poly(butyl-co-vinyl alcohol co-vinyl acetate). As another example, a copolymeric fourth water-soluble polymer may comprise acrylic acid, methyl acrylate, methyl methacrylate, or methacrylic acid monomers. As yet another example, in some embodiments a copolymeric fourth water soluble polymer comprises a urethane. For example, the fourth water soluble polymer could comprise polyethylene glycol co-polymerized with methylene diphenyl isocyanate and/or hydrogenated methylene diphenyl diisocyanate (e.g., Lubrizol Tecophilic® or a generic equivalent thereof). And of course, it should be understood that a comparatively more hydrophobic fourth water-soluble polymer need not be a copolymer. For example, depending on the embodiment, fourth water-soluble polymer comprises poly(propylene glycol), poly(ethylene glycol), or poly(ethylene oxide).

Where the polymer of the coating (e.g., the fourth water soluble polymer) is a copolymer (e.g., poly(vinyl alcohol-co-vinyl acetate) or poly(butyl-co-vinyl alcohol co-vinyl acetate)), the fourth water soluble polymer may comprise vinyl alcohol monomers in any of a variety of suitable weight percentages versus the weight of the fourth water soluble polymer as a whole. In some embodiments, a fourth water soluble polymer comprises vinyl alcohol monomers in an amount of greater than or equal to 10 wt %, greater than or equal to 15 wt %, greater than or equal to 20 wt %, greater than or equal to 25 wt %, greater than or equal to 30 wt %, greater than or equal to 35 wt %, greater than or equal to 40 wt %, greater than or equal to 45 wt %, greater than or equal to 50 wt %, greater than or equal to 55 wt %, greater than or equal to 60 wt %, greater than or equal to 65 wt %, greater than or equal to 70 wt %, greater than or equal to 75 wt %, greater than or equal to 80 wt %, greater than or equal to 85 wt %, greater than or equal to 90 wt %, or greater than or equal to 95 wt % versus the weight of the fourth water soluble polymer. In some embodiments, a fourth water soluble polymer comprises vinyl alcohol monomers in an amount of less than or equal to 97 wt %, less than or equal to 95 wt %, less than or equal to 90 wt %, less than or equal to 85 wt %, less than or equal to 80 wt %, less than or equal to 75 wt %, less than or equal to 70 wt %, less than or equal to 65 wt %, less than or equal to 60 wt %, less than or equal to 55 wt %, less than or equal to 50 wt %, less than or equal to 45 wt %, less than or equal to 40 wt %, less than or equal to 35 wt %, less than or equal to 30 wt %, less than or equal to 25 wt %, less than or equal to 20 wt %, or less than or equal to 15 wt % versus the weight of the fourth water soluble polymer. Combinations of these ranges are also possible (e.g., greater than or equal to 10 wt % and less than or equal to 97 wt %, greater than or equal to 10 wt % and less than or equal to 90 wt %, or greater than or equal to 20 wt % and less than or equal to 80 wt %). Other ranges, both higher and lower than those described above, are also possible, as the disclosure is not so limited.

Where the polymer of the coating (e.g., the fourth water soluble polymer) is a copolymer (e.g., poly(vinyl alcohol-co-vinyl acetate) or poly(butyl-co-vinyl alcohol co-vinyl acetate)), the fourth water soluble polymer may comprise vinyl acetate monomers in any of a variety of suitable weight percentages versus the weight of the fourth water soluble polymer as a whole. In some embodiments, a fourth water soluble polymer comprises vinyl acetate monomers in an amount of greater than or equal to 3 wt %, greater than or equal to 5 wt %, greater than or equal to 10 wt %, greater than or equal to 15 wt %, greater than or equal to 20 wt %, greater than or equal to 25 wt %, greater than or equal to 30 wt %, greater than or equal to 35 wt %, greater than or equal to 40 wt %, greater than or equal to 45 wt %, greater than or equal to 50 wt %, greater than or equal to 55 wt %, greater than or equal to 60 wt %, greater than or equal to 65 wt %, greater than or equal to 70 wt %, greater than or equal to 75 wt %, greater than or equal to 80 wt %, or greater than or equal to 85 wt %. In some embodiments, a fourth water soluble polymer comprises vinyl acetate monomers in an amount of less than or equal to 90 wt %, less than or equal to 85 wt %, less than or equal to 80 wt %, less than or equal to 75 wt %, less than or equal to 70 wt %, less than or equal to 65 wt %, less than or equal to 60 wt %, less than or equal to 55 wt %, less than or equal to 50 wt %, less than or equal to 45 wt %, less than or equal to 40 wt %, less than or equal to 35 wt %, less than or equal to 30 wt %, less than or equal to 25 wt %, less than or equal to 20 wt %, less than or equal to 15 wt %, less than or equal to 10 wt %, or less than or equal to 5 wt %. Combinations of these ranges are also possible (e.g., greater than or equal to 3 wt % and less than or equal to 90 wt %, greater than or equal to 10 wt % and less than or equal to 90 wt %, or greater than or equal to 20 wt % and less than or equal to 80 wt %). Other ranges, both higher and lower than those described above, are also possible, as the disclosure is not so limited.

It should, of course, be understood that for poly(vinyl alcohol-co-vinyl acetate), the total weight percentage of vinyl alcohol and vinyl acetate molecules will total approximately 100 wt %, meaning that ranges from the above paragraphs may be linked under such circumstances. However, it should also be understood that the above-mentioned ranges are generally independent and it should be recognized that vinyl alcohol and vinyl acetate concentrations may, in some cases, be drawn from the above-mentioned ranges independently with the proviso that the total monomer content of vinyl alcohol and vinyl acetate is less than or equal to 100%. For example, ranges in the preceding paragraphs may not be linked for poly(butyl-co-vinyl alcohol co-vinyl acetate)), where the total vinyl alcohol and vinyl acetate totals less than 100% and the butyl-alcohol monomers contribute the approximate balance of the 100%.

In some embodiments, a coating provided herein provides a visible indication of its presence (e.g., because an indicator is homogeneously added to the coating or is selectively patterned into the coating). Any of a variety of suitable indicators may be used, including copper phthalocyanine (with any of a variety of suitable carbon: chloride ratios and/or copper: chloride ratios as discussed below). Other suitable indicators include, but are not limited to, carbon black, carbazole violet, reactive black 5, titanium dioxide, barium sulfate, and/or any of a variety of suitable other pigments or dyes. For instance, in some embodiments, the dye is a reactive dye. Non-limiting examples of suitable reactive dyes include tetrasodium; 4-amino-5-hydroxy-3,6-bis[[4-(2-sulfonatooxyethylsulfonyl)phenyl]diazenyl]naphthalene-2,7-disulfonate (Reactive Black 5), copper; 33-[[4-(2-hydroxyethylsulfonyl)phenyl]sulfamoyl]-2,11,20,29,39,40-hexaza-37,38-diazanidanonacyclo[28.6.1.13,10.112,19.121,28.04,9.013,18.022,27.031,36]tetraconta-1,3(40),4(9),5,7,10,12(39),13(18),14,16,19,21,23,25,27,29,31(36),32,34-nonadecaene-6,15,24-trisulfonic acid (Reactive Blue 21), 2-Naphthalenesulfonicacid,7-(acetylamino)-4-hydroxy-3-[[4-[[2-(sulfooxy)ethyl]sulfonyl]phenyl]azo]-, disodium salt (9CI) (Reactive Orange 78), Reactive Yellow 15, Disodium 1-amino-9,10-dioxo-4-[(3-{[2-(sulfonatooxy)ethyl]sulfonyl}phenyl)amino]-9,10-dihydro-2-anthracenesulfonate (Reactive Blue 19), 1-Amino-4-[3-(4,6-dichlorotriazin-2-ylamino)-4-sulfophenylamino]anthraquinone-2-sulfonic acid (Reactive Blue 4), C.I. Reactive Red 11, 4-[2-(5-carbamoyl-1-ethyl-4-methyl-2,6-dioxopyridin-3-ylidene)hydrazinyl]-6-[(4,6-dichloro-1,3,5-triazin-2-yl)amino]benzene-1,3-disulfonate (C.I. Reactive Yellow 86), Tetrasodium 6,13-dichloro-3,10-bis [[4-[(4,6-dichloro-1,3,5-triazin-2-yl) amino] sulphonatophenyl] amino] triphenodioxazinedisulphonate (C.I. Reactive Blue 163), and/or 5-(benzoylamino)-4-hydroxy-3-[[1-sulfo-6-[[2-(sulfooxy)ethyl]sulfonyl]-2-naphthalenyl]azo]-, tetrasodium salt (C.I. Reactive Red 180).

In some embodiments, the coating comprises a non-reactive dye, pigment, and/or radiopacifier. Non-limiting examples of suitable non-reactive dyes include: phthalocyanine blue, phthalocyanine green, carbazole violet, C.I. Vat Orange 1, 2-[[2,5-Diethoxy-4-[(4-methylphenyl)thiol]phenyl]azo]-1,3,5-benzenetriol, 16,23-Dihydrodinaphtho[2,3-a: 2′,3′-i] naphth [2′,3′: 6,7] indolo [2,3-c] carbazole-5,10,15,17,22,24-hexone, N, N'-(9,10-Dihydro-9,10-dioxo-1,5-anthracenediyl) bisbenzamide, 7,16-Dichloro-6,15-dihydro-5,9,14,18-anthrazinetetrone, 16,17-Dimethoxydinaphtho [1,2,3-cd: 3′,2′,1′-1m] perylene-5,10-dione, 4-[(2,4-dimethylphenyl)azo]-2,4-dihydro-5-methyl-2-phenyl-3H-pyrazol-3-one, 6-Ethoxy-2-(6-ethoxy-3-oxobenzo[b]thien-2(3H)-ylidene) benzo[b]thiophen-3 (2H)-one, Disodium 1-amino-4-[[4-[(2-bromo-1-oxoallyl)amino]-2-sulfonatophenyl]amino]-9,10-dihydro-9,10-dioxoanthracene-2-sulfonate, and combinations hereof. Non-limiting examples of suitable non-reactive pigments include: carbon black, modified carbon black, titanium dioxide, chromium-cobalt-aluminum oxide, chromium oxide greens, iron oxides, mica-based pearlescent pigments, and combination thereof. Non-limiting examples of radiopaque dyes include platinum, palladium, bismuth oxychloride, bismuth subcarbonate, tantalum, barium sulfate, silver, gold, silver sulfadiazine, titanium dioxide, and iodine based compounds such as Omnipaque. In some embodiments, the marking comprises a fluorescent dye (e.g., Fluorescein isothiocyanate (FIT-C), fluorescein-N-hydroxysuccinimide, eosin Y, and the like).

In some embodiments, the coating comprises a salt. Non-limiting examples of suitable salts include phosphates (e.g., MSP, DSP, TSP), borates, sodium chloride, citrates, ethylenediaminetetraacetates, sulfites, sulfates, hyposulfites, metal oxides, selenium dioxide, selenium trioxide, selenous acid, selenic acid, nitrates, silicates, and botanic acid.

In some embodiments, the coatings comprise a TPU pad printing ink, such as Tampa® Pur 980 Black TPU and/or Tampa® Star 980 Black TPR, Marabu GmbH & Co.

In some embodiments, the coating may comprise a suitable ratio of carbon: chloride ratios. By way of example, and without wishing to be bound by such, copper pthalocyanine, if used, may have any of a variety of suitable carbon: chloride ratios. In some embodiments, the carbon: chloride ratio is greater than or equal to 15:6,greater than or equal to 15:5, greater than or equal to 15:4, greater than or equal to 15:3,or greater than or equal to 15:2. In some embodiments, the carbon: chloride ratio is greater than or equal to 15:1, less than or equal to 15:2, less than or equal to 15:3, less than or equal to 15:4, or less than or equal to 15:5. Combinations of these ranges are also possible (e.g., greater than or equal to 15:6 and less than or equal to 15:1). Other ranges, both higher and lower than those described above, are also possible, as the disclosure is not so limited.

Likewise, the copper pthalocyanine, if used, may have any of a variety of suitable copper: chloride ratios. In some embodiments, the copper: chloride ratio is less than or equal to 4:1, less than or equal to 3:1, less than or equal to 2:1, or less than or equal to 15:14. In some embodiments, the copper: chloride ratio is greater than or equal to 1:1, greater than or equal to 15:14, greater than or equal to 2:1, greater than or equal to 3:1. Combinations of these ranges (e.g., greater than or equal to 1:1 and less than or equal to 4:1) are also possible. As would be understood by one of ordinary skill in the art, other compounds having suitable copper: chloride ratios are also possible.

In some embodiments, the total weight of the first water soluble polymer and another (e.g., a third) water soluble polymer in the polymeric tube is greater than or equal to 20 w/w%, greater than or equal to 25 w/w%, greater than or equal to 30 w/w%, greater than or equal to 35 w/w%, greater than or equal to 40 w/w%, greater than or equal to 45 w/w%, greater than or equal to 50 w/w%, greater than or equal to 55 w/w%, greater than or equal to 60 w/w%, greater than or equal to 65 w/w%, greater than or equal to 70 w/w%, greater than or equal to 75 w/w%, greater than or equal to 80 w/w%, greater than or equal to 85 w/w%, greater than or equal to 90 w/w%, greater than or equal to 95 w/w%, greater than or equal to 98 w/w%, or greater than or equal to 99 w/w% at an equilibrium water content state. In some embodiments, the total weight of the first water soluble polymer and another (e.g., a third) water soluble polymer in the polymeric tube in an amount of less than or equal to 100 w/w%, less than or equal to 90 w/w%, less than or equal to 98 w/w%, less than or equal to 95 w/w%, less than or equal to 90 w/w%, less than or equal to 85 w/w%, less than or equal to 80 w/w%, less than or equal to 75 w/w%, less than or equal to 70 w/w%, less than or equal to 65 w/w%, less than or equal to 60 w/w%, less than or equal to 55 w/w%, less than or equal to 50 w/w%, less than or equal to 45 w/w%, less than or equal to 40 w/w%, less than or equal to 35 w/w%, less than or equal to 30 w/w%, or less than or equal to 25 w/w% at an equilibrium water content state. Combinations of the above-referenced ranges are also possible (e.g., greater than or equal to 20 w/w% and less than or equal to 100 w/w%). Other ranges are also possible.

In some embodiments, the ratio of the first water soluble polymer to the third water soluble polymer present in the polymeric tube is less than or equal to 100:0, less than or equal to 99:1, less than or equal to 95:5, less than or equal to 90:10, less than or equal to 80:20, less than or equal to 70:30, less than or equal to 60:40, or less than or equal to 55:45. In some embodiments, the ratio of the first water soluble polymer to the third water soluble polymer present in the polymeric tube is greater than or equal to 50:50, greater than or equal to 60:40, greater than or equal to 70:30, greater than or equal to 80:20, greater than or equal to 90:10, greater than or equal to 95:5, or greater than or equal to 99:1. Combinations of the above-referenced ranges are also possible (e.g., less than or equal to 100:0 and greater than or equal to 50:50). Other ranges are also possible.

As described above and herein, in some embodiments, the polymeric tube (e.g., polymeric tube 12 of FIG. 5B, polymeric tube 14 of FIG. 5C) comprises a second water soluble polymer (e.g., second water soluble polymer 40) disposed within at least a portion of the plurality of pores (e.g., plurality of pores 30) of the body portion (e.g., body portion 20 comprising or formed of a polymeric material). In some embodiments, the second water soluble polymer comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In some embodiments, the second water soluble polymer is poly(acrylic acid). The second water soluble polymer may have a different chemical composition from that of the first (e.g., and optionally third) water soluble polymers.

The second water soluble polymer (e.g., second water soluble polymer 40) may be present in the polymeric tube in any suitable amount. For example, in some embodiments, the second water soluble polymer is present in the polymeric tube in an amount of greater than or equal to 0.05 w/w%, greater than or equal to 0.1 w/w%, greater or than or equal to 0.2 w/w%, greater than or equal to 0.5 w/w%, greater than or equal to 1.0 w/w%, greater than or equal to 2.0 w/w%, greater than or equal to 3.0 w/w%, greater than or equal to 4.0 w/w%, greater than or equal to 5.0 w/w%, greater than or equal to 10 w/w%, greater than or equal to 20 w/w%, greater than or equal to 30 w/w%, greater than or equal to 40 w/w%, greater than or equal to 50 w/w%, greater than or equal to 60 w/w%, greater than or equal to 70 w/w%, greater than or equal to 80 w/w%, or greater than or equal to 90 w/w% at an equilibrium water content state. In some embodiments, the second water soluble polymer 40 is present in the polymeric tube in an amount of less than or equal to 95 w/w%, less than or equal to 90 w/w%, less than or equal to 80 w/w%, less than or equal to 70 w/w%, less than or equal to 60 w/w%, less than or equal to 50 w/w%, less than or equal to 40 w/w%, less than or equal to 30 w/w%, less than or equal to 20 w/w%, less than or equal to 10 w/w%, less than or equal to 5.0 w/w%, less than or equal to 4.0 w/w%, less than or equal to 3.0 w/w%, less than or equal to 2.0 w/w%, less than or equal to 1.0 w/w%, less than 0.5 w/w%, less than 0.2 w/w%, or less than 0.1 w/w% at an equilibrium water content state. In some embodiments, 0 w/w% of the second water soluble polymer is present. Combinations of the above-referenced ranges are also possible (e.g., greater than or equal to 0.05 w/w% and less than or equal to 95 w/w%). Other ranges are also possible.

In some embodiments, the water-soluble polymer (e.g., the first water soluble polymer, the second water soluble polymer, the third water soluble polymer, the fourth water soluble polymer) has a particular molecular weight. In some embodiments, the molecular weight of the water soluble polymer (e.g., each, independently, the first water soluble polymer, the second water soluble polymer, or the third water soluble polymer) may be greater than or equal to 40 kDa, greater than or equal to 50 kDa, greater than or equal to 75 kDa, greater than or equal to 100 kDa, greater than or equal to 125 kDa, greater than or equal to 150 kDa, greater than or equal to 175 kDa, greater than or equal to 200 kDa, greater than or equal to 250 kDa, greater than or equal to 300 kDa, greater than or equal to 350 kDa, greater than or equal to 400 kDa, greater than or equal to 450 kDa, greater than or equal to 500 kDa, greater than or equal to 600 kDa, greater than or equal to 700 kDa, greater than or equal to 800 kDa, greater than or equal to 900 kDa, greater than or equal to 1000 kDa, greater than or equal to 1500 kDa, greater than or equal to 2000 kDa, greater than or equal to 3000 kDa, or greater than or equal to 4000 kDa. In some embodiments, the molecular weight of the water soluble polymer (e.g., each, independently, the first water soluble polymer, the second water soluble polymer, or the third water soluble polymer) may be less than or equal to 5000 kDa, less than or equal to 4000 kDa, less than or equal to 3000 kDa, less than or equal to 2000 kDa, less than or equal to 1500 kDa, less than or equal to 1000 kDa, less than or equal to 900 kDa, less than or equal to 800 kDa, less than or equal to 700 kDa, less than or equal to 600 kDa, less than or equal to 500 kDa, less than or equal to 450 kDa, less than or equal to 400 kDa, less than or equal to 350 kDa, less than or equal to 300 kDa, less than or equal to 250 kDa, less than or equal to 200 kDa, less than or equal to 175 kDa, less than or equal to 150 kDa, less than or equal to 125 kDa, less than or equal to 100 kDa, less than or equal to 75 kDa, or less than or equal to 50 kDa. Combinations of the above-referenced ranges are also possible (e.g., a molecular weight of greater than or equal to 40 kDa and less than or equal to 5000 kDa). Other ranges are also possible.

In some embodiments, the biologically active agent is present in the bulk polymeric material formed as a layer in the polymeric tube. For example, in some embodiments, the polymeric material comprises a first surface and a second surface wherein the first surface and/or the second surface may be coated. In some embodiments, the first surface and/or the second surface is coated with a polymer, a second biologically active agent (the same or different from the biologically active agent present in the polymeric material), or combinations thereof. In some embodiments, the polymeric tube comprises two or more layers of polymeric material in the body portion. In some embodiments, each layer of polymeric material may comprise the same, comprising different, or comprise no biologically active agent. In an illustrative embodiment, the body portion of a polymeric tube comprises a first polymeric material layer comprising a first biologically active agent and a second polymeric material layer disposed on the first polymeric material layer, comprising a second biologically active agent. Other combinations of layers are also possible.

In some embodiments, the biologically active agent is distributed within the polymeric material (of the body portion) and/or the first water soluble polymer substantially homogeneously. For example, in some embodiments, the amount of the biologically active agent does not vary by more than 50% at a given arbitrary section (e.g., section 52, section 54 in FIG. 5A) across a cross-sectional area of the body portion and/or first water soluble polymer as compared to an average amount of the biologically active agent in the body portion and/or first water soluble polymer.

In some embodiments, the biologically active agent is distributed within the polymeric material (e.g., polymeric tube) non-homogeneously (i.e., on one or more surfaces of the polymeric material). For example, in some embodiments, the amount of the biologically active agent varies by more than 50% at a given arbitrary section (e.g., section 52, section 54 in FIG. 5A) across a cross-sectional area of the body portion and/or first water soluble polymer as compared to an average amount of the biologically active agent in the body portion and/or first water soluble polymer.

In some embodiments, as described in more detail above, the body portion (e.g., the polymeric material) may comprise a plurality of pores. The polymeric material of the body portion may comprise a first water soluble polymer as described herein. In some embodiments, the biologically active agent is distributed within the polymeric material (e.g., the first water soluble polymer) homogeneously or non-homogeneously to within one of the above-noted ranges, but not within the plurality of pores. That is to say, in some embodiments, the plurality of pores may be substantially devoid of the biologically active agent. In some embodiments, the plurality of pores may comprise a second biologically active agent, the same or different than a (first) biologically active agent present within the polymeric material forming the bulk of the polymeric tube (e.g., the polymeric material comprising a first water soluble polymer). In yet other embodiments, the biologically active agent is present only in the plurality of pores.

A variety of methods may be used to spatially vary the loading of the biologically active ingredient, depending on the embodiment, to produce a first portion of a polymeric material (e.g., a polymeric tube) that has a first loading of the biologically active ingredient and a second portion of the polymeric material that has second loading of the biologically active ingredient. Spatial variation in the loading of the biologically active ingredients may, in some embodiments, vary with one or more physical properties of the polymeric material such that, for example, a first portion of a polymeric material that has a higher porosity than a second portion of the polymeric material also contains a greater amount of the biologically active agent (e.g., if the biologically active agent is contained within pores of the polymeric material) or a lesser amount of the biologically active agent (e.g., if the biologically active agent is contained within the bulk polymeric material).

Suitable biologically active agents are described in more detail below and include, for example, pharmaceutical agents (e.g., drugs), calcium salt (e.g., calcium chloride), iron salt (e.g. ferrous sulfate), starch, modified silica, cellulose, amongst others. The term “biologically active agent” as used herein generally refers to an agent which, when administered to a subject, has a physiologically significant effect on at least a portion of the body of the subject.

In some embodiments, one or more biologically active agents may be distributed within body portion 20 and/or plurality of pores 30 (FIGS. 5B-5C). In some embodiments, the biologically active agent is a therapeutic agent. As used herein, the term “therapeutic agent” or also referred to as a “drug” refers to an agent that is administered to a subject to treat, reduce, delay, ameliorate and/or prevent a disease, disorder, or other clinically recognized condition, or for prophylactic purposes, and, in some embodiments, has a clinically significant effect on the body of the subject to treat, reduce, delay, ameliorate and/or prevent the disease, disorder, or condition. Therapeutic agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. In some embodiments, the therapeutic agent may be selected from “Approved Drug Products with Therapeutic Equivalence and Evaluations,” published by the United States Food and Drug Administration (F.D. A.) (the “Orange Book”). In some cases, the therapeutic agent is one that has already been deemed safe and effective for use in humans or animals by the appropriate governmental agency or regulatory body. For example, drugs approved for human use are listed by the FDA under 21 C.F. R. §§ 330.5, 331 through 361, and 440 through 460, incorporated herein by reference; drugs for veterinary use are listed by the FDA under 21 C.F. R. §§ 500 through 589, incorporated herein by reference. All listed drugs are considered acceptable for use in accordance with the present invention. In some embodiments, the therapeutic agent is a small molecule. Exemplary classes of agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents (e.g., taxanes, such as paclitaxel and docetaxel; cisplatin, doxorubicin, methotrexate, etc.), antihistamines, antiplatelet agents, antineoplastic agents, antiseptic agents, antimigraine drugs, hormones, prostaglandins, antimicrobials (including antibiotics, antifungals, antivirals, antiparasitics), antimuscarinics, anxiolytics, bacteriostatics, biologic entities, immunosuppressant agents, sedatives, hypnotics, antipsychotics, bronchodilators, anti-asthma drugs, cardiovascular drugs, cannabanoids, anesthetics, anti-coagulants, inhibitors of an enzyme, steroidal agents, steroidal or non-steroidal anti-inflammatory agents, coagulative agentscorticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

It should be appreciated that where more than one biologically active agent is present (e.g., a first biologically active agent present in the polymeric material forming the bulk of the body portion, or a second biologically active agent in the pores of the body portion), each biologically active agent may independently be one of the active agents described above.

The biologically active agent (e.g., first, second biologically active agent) may be distributed within the body portion and/or the polymeric material and present in the polymeric tube in any suitable amount.

Advantageously, the polymeric tubes described herein may permit higher concentrations (weight percent) of active agents such as biologically active agents to be incorporated into the polymeric tubes as compared to certain other polymeric tubes (e.g., certain polymeric tubes including solely a coating of the biologically active agent). In some embodiments, the biologically active agent is associated with the first water soluble polymer and/or the second water soluble polymer. In some embodiments, the biologically active agent is dispersed within the first water soluble polymer and/or the second water soluble polymer. Additionally, or alternatively, the polymeric tubes described herein may permit extended release of one or more biologically active agents compared to certain other polymeric tubes (e.g., certain polymeric tubes including solely a coating of the biologically active agent).

Like loading of the biologically active agent, the rate of release of a biologically active agent may be varied spatially such that a first portion of a polymeric material (e.g., polymeric tube) releases the biologically active agent at a different rate than a second portion of the polymeric material. The release rate may be varied by establishing spatial variation in loading of the polymeric material as discussed in greater detail above, and/or by varying one or more physical properties of the polymeric material in order to modulate the release rate. For example, in some embodiments, biologically active material may be released at different rates from materials with different porosities (e.g., if the porosity provides for faster transport of the biologically active agent out of the material) or from materials with spatially varying diffusion coefficients (e.g., because of different crystallinity, anisotropy, and/or other physical properties that may be spatially varied within the polymeric material). Thus, in some embodiments a first release rate of a biologically active agent (e.g., a therapeutic agent) from a first portion of a polymeric material (e.g., polymeric tube) is different than a second release rate of the biologically active agent from a second portion of the polymeric material.

In some embodiments, the biologically active agent may be released from the body portion of the polymeric tube by any suitable means. In some embodiments, the biologically active agent is released by diffusion out of the body portion (e.g., the polymeric material of the body portion). In some embodiments, the biologically active agent is released by degradation of at least a portion of the body portion (e.g., biodegradation, enzymatic degradation, hydrolysis of the polymeric material forming the body portion, or of the polymeric material in the pores of the body portion). In some embodiments, the active substance is released from the polymeric tube at a particular rate. Those skilled in the art would understand that the rate of release may be dependent, in some embodiments, on the solubility of the biologically active agent in the medium in which the polymeric tube is exposed, such as a physiological fluid such as blood. In some embodiments, the release rate may be dependent on the cross-link density, porosity, pore size distribution, pore interconnectivity (e.g., tortuosity), crystallinity, and/or number of biologically active agent containing layers, in the polymeric tube (e.g., the body portion of the polymeric tube).

In some embodiments, the polymeric tube may be configured to release one or more biologically active agent(s) using a combination of burst release(s) and controlled release(s). In an illustrative example, a biologically active agent may be released by a first burst release followed by a controlled release. In another illustrative example, a first biologically active agent may be released by a burst release and a second biologically active agent may be released at a particular average rate. In some embodiments, the first biologically active agent and the second biologically active agent may begin release at substantially the same time. In some embodiments, the first biologically active agent and the second biologically active agent may be released at different times.

The polymeric tubes, articles, devices, catheters, kits, and methods described herein may be administered to any suitable subject. The term “subject”, as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the polymeric tube, article, or device.

These materials can be made as tough, high strength materials having lubricious and biocompatible surfaces. Nanoporous and microporous solids are described herein that have a particularly high Young's modulus and tensile strength. A nanoporous material is a solid that contains interconnected pores of up to 100 nm in diameter. Processes for making hydrogels are also described. Hydrophilic polymers may be used to make these various porous solids so that a hydrophilic solid is obtained. The water content of a nanoporous or a microporous solid can be high, e.g., 50% w/w at EWC. The water content of a hydrogel may be higher, for example, up to 90% w/w in principle. The porous solid materials can be used to make various polymeric tubes, articles, or devices, including medical catheters and implants with significant reductions in adsorption and/or adhesion of biological components to their surfaces.

These or other porous materials may be processed to include polymers that are bulk-incorporated into pores of the solid. An embodiment of the material is a porous material comprising water soluble polymers entrapped in pores of the material. Polymers entrapped by this method have been observed to be present in the pores and to remain in the pores after repeated hydration and dehydration. The entrapped polymers provide a surface that is scratch-resistant and effectively permanent, with the incorporated polymer providing desirable properties beyond the outer surface of the material. In aqueous medium, hydrophilic polymers entrapped by this method are hydrated to extend beyond the surface to enhance biocompatibility and lubricity.

Processes for making the material are described in International Patent Application Publication Nos. WO2018/237166 and WO2017/112878, which are hereby incorporated by reference in their entirety. Processes for making the material can include extrusion so that polymeric tubes with a high aspect ratio may be created. An embodiment of a process for making the materials involves heating a mixture that comprises at least one water soluble polymer and a solvent to a temperature above the melting point of the polymer solution forming the mixture in a solvent-removing environment resulting in a crosslinked matrix and continuing to remove the solvent until the crosslinked matrix is a microporous or a nanoporous solid material. The crosslinking can take place while cooling the mixture and/or in the solvent-removing environment. Further polymers may be incorporated into pores of the material.

Disclosed herein are forming processes, including extrusion, to make a high strength porous solid. Guidance as to processes and parameters to make porous solids are disclosed, as well as the porous solids. Guidance for bulk incorporation of polymers into porous solids is disclosed. Porous solids are disclosed with good properties and the further inclusion of bulk incorporated polymers provides further improvements.

Herein is disclosed a new process that provides for extrusion of high strength materials. Some embodiments of the process provide one or more of: removal of a solvent from a hydrophilic polymer-solvent mixture as the material is extruded, extruding at a cold temperature, extruding into a solvent-removing environment, and further removal of solvent for a period of time after extrusion. Further, an annealing phase and/or a bulk incorporation for further polymers phase may also be included.

FIGS. 5D-5F depict an embodiment of an apparatus to make the porous solid materials. A polymeric tube 100 as depicted includes a syringe pump 102 to accept at least one syringe 104, an optional heating jacket (not shown) to heat the syringes, die head 106, heating element 108 and power cables 109 for the same, providing heating as needed for die head 106 (detail not shown in FIG. 5D), dispensing spool 110 for core tubing 112, uptake spool 114 and motor (not shown) for core tubing, bath 116 for the extruded material 117, with the bath having temperature control for cooling or heating, depicted as heat exchanger 118 that comprises heat exchanging pipe 120 in bath 116. Die head 106 accepts the core tubing 110 which passes therethrough. Feed line 122 from the syringes to die head 106 provides a feed to polymeric tube 100. A system for this embodiment may further include a weigh station, a jacketed vessel for heating and mixing solutions for loading into the syringes, and a solvent-removal environment for further drying of tubing removed from bath 116. The system may also have a heating station for annealing the tubing or other extrusion product with heat when desired. Core tubing made of PTFE as well as wires, air, gas, non-solvent liquid or other materials may be used for a core.

In use, by way of example, a polymer is heated in a suitable solvent in a jacketed vessel and placed into syringe 104. One or more polymers may be present and a radiopaque agent or other additive may be added. One or more syringes may be used with the same or different mixtures. The syringe(s) of the polymer are heated to a predetermined temperature, e.g., of no more than 80-95° C., and degassed before extrusion. Syringe 104 is mounted on syringe pump 102 with a wrap heater to maintain temperature during extrusion. Core 112 is looped through die head 106, e.g., a heated out-dwelling die head, which feeds into extrusion bath 116, and then attached to an uptake spool 114 that is driven by a motor. The temperature of the bath is controlled using heat exchanger 118, such as a chiller; extruded materials may be extruded at temperatures ranging from −30° C. to 75° C. other temperatures may be used, and 0° C. is a generally useful temperature setting for extrusion. Artisans will immediately appreciate that all ranges and values between the explicitly stated bounds are contemplated, with, e.g., any of the following being available as an upper or lower limit: −30, −25, −20, −15, −10, −5, 0, 5, 10, 15, 20, 25, 30, 35, 40, 45, 50, 55, 60, 65, 70, 75° C. Uptake (e.g., puller) spool 114 motor speed can be controlled to adjust outer diameter gauge size around core 112. Adjusting die size, material feed rate, tubing core diameter, and puller speed play roles in adjusting final tubing gauge, e.g., in embodiments wherein a catheter is made. Polymer feed rates are adjustable, e.g., by control of syringe pump 102 in this embodiment. Connectors 122 join the one or more syringes to die head 106. Many pumps and other tools for controllably feeding a polymer solution are known. The apparatus and method can be adapted for a drawing process although alternative feed processes are available.

In some embodiments, a composition (e.g., a pre-polymer composition) may be provided (e.g., for extrusion) prior to formation of the polymeric material. In some embodiments, the composition comprises an aqueous solution. The aqueous solution can comprise an osmotic agent at a concentration of greater than or equal to 0.01 M and less than or equal to 8 M. The aqueous solution can comprise a radiopaque agent in an amount of greater than or equal to 0 w/w% and less than or equal to 50 w/w% (e.g., less than or equal to 40 w/w%). The composition can further comprise a water-soluble polymer having a molecular weight of greater than or equal to 40 kDa and less than or equal to 5000 kDa, and present in the solution in an amount greater than or equal to 10 w/w% and less than or equal to 50 w/w%.

In some embodiments, the composition forms a swellable polymeric material upon extrusion.

In some embodiments, the osmotic agent is present in the solution at a concentration of greater than or equal to 0.01 M, greater than or equal to 0.1 M, greater than or equal to 0.5 M, greater than or equal to 1 M, greater than or equal to 2 M, greater than or equal to 3 M, greater than or equal to 4 M, greater than or equal to 5 M, or greater than or equal to 6 M. In some embodiments, the osmotic agent is present in the solution at a concentration of less than or equal to 8 M, less than or equal to 6 M, less than or equal to 4 M, less than or equal to 2 M, less than or equal to 1 M, less than or equal to 0.5 M, or less than or equal to 0.1 M. Combinations of the above-referenced ranges are also possible (e.g., greater than or equal to 0.01 M and less than or equal to 8 M). Osmotic agents are described in more detail herein.

In some embodiments, the radiopaque agent is present in the solution in an amount of greater than or equal to 0 w/w%, greater than or equal to 5 w/w%, greater than or equal to 10 w/w%, greater than or equal to 15 w/w%, greater than or equal to 20 w/w%, greater than or equal to 25 w/w%, greater than or equal to 30 w/w%, greater than or equal to 35 w/w%, greater than or equal to 40 w/w%, or greater than or equal to 45 w/w%. In some embodiments, the radiopaque agent is present in the solution in an amount less than or equal to 50 w/w%, less than or equal to 45 w/w%, less than or equal to 40 w/w%, less than or equal to 35 w/w%, less than or equal to 30 w/w%, less than or equal to 25 w/w%, less than or equal to 20 w/w%, less than or equal to 15 w/w%, less than or equal to 10 w/w%, or less than or equal to 5 w/w%. Combinations of the above-referenced ranges are also possible (e.g., greater than or equal to 0 w/w% and less than or equal to 50 w/w%). Other ranges are also possible. Radiopaque agents are described in more detail, below.

In some embodiments, the water-soluble polymer is present in the solution in an amount greater than or equal to 10 w/w%, greater than or equal to 13 w/w%, greater than or equal to 15 w/w%, greater than or equal to 20 w/w%, greater than or equal to 25 w/w%, greater than or equal to 30 w/w%, greater than or equal to 35 w/w%, greater than or equal to 40 w/w%, or greater than or equal to 45 w/w%. In some embodiments, the water-soluble polymer is present in the solution in an amount less than or equal to 50 w/w%, less than or equal to 45 w/w%, less than or equal to 40 w/w%, less than or equal to 35 w/w%, less than or equal to 30 w/w%, less than or equal to 25 w/w%, less than or equal to 20 w/w%, less than or equal to 15 w/w%, or less than or equal to 13 w/w%. Combinations of the above-referenced ranges are also possible (e.g., greater than or equal to 10 w/w% and less than or equal to 50 w/w%). In some embodiments, the water-soluble polymer is present in the solution in an amount greater than or equal to 13 w/w%.

In some embodiments, the method for forming the polymeric materials and/or polymeric tubes described herein comprises providing a mixture comprising a first water soluble polymer and an osmotic agent (e.g., a salt) as described above. In some embodiments, the mixture is extruded. In some embodiments, the extruded mixture is extruded on a core material to form the polymeric material disposed on the core material. In some embodiments, the formed polymeric material is exposed to a non-solvent of the polymeric material. In some embodiments, a solution comprising a second water soluble polymer different that the first water soluble polymer and, optionally, an osmotic agent, is introduced to the polymeric material. In some embodiments, the polymeric material (e.g., after introducing the solution to the osmotic agent) is heated. In some embodiments, the solution is flowed against the polymeric material. In some embodiments, the polymeric material may be dried.

In an exemplary set of embodiments, the method for forming the polymeric materials and/or polymeric tubes described herein comprises providing a mixture comprising a first water soluble polymer and an osmotic agent (e.g., a salt), wherein the first water soluble polymer is present in the mixture in an amount greater than or equal to 10 w/w% (e.g., greater than or equal to 13 w/w% or greater than or equal to 13 w/w% and less than or equal to 50 w/w%) versus the total weight of the mixture, performing the steps of: extruding the mixture at a temperature greater than or equal to 65° C. (e.g., greater than or equal to 65° C. and less than or equal to 100° C.) at atmospheric pressure, on a core material to form the polymeric material disposed on the core material (e.g., a solid rod or a gas), exposing the polymeric material to a non-solvent of the polymeric material at a temperature less than or equal to 28° C. (e.g., less than or equal to 28° C. and greater than or equal to −20° C.) for greater than or equal to 15 minutes (e.g., greater than or equal to 1 hour and less than or equal to 240 hours), introducing, to the polymeric material, a solution comprising a biologically active agent and/or a second water soluble polymer, different than the first water soluble polymer, and/or an osmotic agent (e.g., a salt), heating the polymeric material and the solution to a temperature of greater than or equal to 25° C. (e.g., greater than or equal to 30° C., or greater than or equal to 30° C. and less than or equal to 65° C.), flowing the solution adjacent the polymeric material, for example, for greater than or equal to 1 hour (e.g., greater than or equal to 1 hour and less than or equal to 48 hours or greater than or equal to 3 hours and less than or equal to 48 hours), and drying the polymeric material. In some embodiments, the biologically active agent is distributed within the polymeric material substantially homogeneously to within less than or equal to 50% of an average loading of the biologically active agent in the polymeric material. In some embodiments, the biologically active agent is distributed within the polymeric material non-homogeneously (i.e., on one or more surfaces of the polymeric material).

In some embodiments, the second water soluble polymer is positioned in at least one pore (or a plurality of pores) of the first water soluble polymer, as described herein.

In some embodiments, the non-solvent comprises alcohol. In some embodiments, the non-solvent is ethanol, methanol, propanol, butanol, pentanol, hexanol, heptanol, octanol, decanol, dodecanol, dimethyl sulfoxide, ethyl acetate, acetates, propionates, ethers, dimethyl formamide, dimethyl acetamide, acetone, acetonitrile, ethylene glycol, propylene glycol, glycerol air, vacuum or combinations thereof. Other non-solvents are also possible (e.g., a solvent having a high solubility to water but a lower solubility to the water-soluble polymer, as compared to the solubility in water).

In some embodiments, the step of extruding the mixture is performed under atmospheric pressure at a temperature of greater than or equal to 65° C., greater than or equal to 70° C., greater than or equal to 75° C., greater than or equal to 80° C., greater than or equal to 85° C., greater than or equal to 90° C., greater than or equal to 95° C., greater than or equal to 100° C., or greater than or equal to 105° C. In some embodiments, the step of extruding the mixture is performed under atmospheric pressure at a temperature of less than or equal to 110° C., less than or equal to 105° C., less than or equal to 100° C., less than or equal to 95° C., less than or equal to 90° C., less than or equal to 85° C., less than or equal to 80° C., less than or equal to 75° C., or less than or equal to 70° C. Combinations of the above-referenced ranges are also possible (e.g., greater than or equal to 65° C. and less than or equal to 110° C.). Other ranges are also possible. Those of ordinary skill in the art would understand, based upon the teachings of this specification, that additional pressures (e.g., greater than atmospheric pressure, less than atmospheric pressure) and/or temperatures are also possible.

In some embodiments, the step of exposing the polymeric material to a non-solvent of the polymeric material is performed at a temperature less than or equal to 28° C., less than or equal to 25° C., less than or equal to 20° C., less than or equal to 15° C., less than or equal to 10° C., less than or equal to 5° C., less than or equal to 0° C., less than or equal to −5° C., less than or equal to −10° C., or less than or equal to −15° C. In some embodiments, the step of exposing the polymeric material to a non-solvent of the polymeric material is performed at a temperature greater than or equal to −20° C., greater than or equal to −15° C., greater than or equal to −10° C., greater than or equal to −5° C., greater than or equal to 0° C., greater than or equal to 5° C., greater than or equal to 10° C., greater than or equal to 15° C., greater than or equal to 20° C., or greater than or equal to 25° C. Combinations of the above-referenced ranges are also possible (e.g., less than or equal to 28° C. and greater than or equal to −20° C.). Other ranges are also possible.

In some embodiments, the step of exposing the polymeric material to the non-solvent of the polymeric material is performed (e.g., at a temperature less than or equal to 28° C. and greater than or equal to −20° C.) for greater than or equal to 1 hour, greater than or equal to 2 hours, greater than or equal to 4 hours, greater than or equal to 6 hours, greater than or equal to 8 hours, greater than or equal to 10 hours, greater than or equal to 15 hours, greater than or equal to 20 hours, greater than or equal to 30 hours, greater than or equal to 40 hours, greater than or equal to 50 hours, greater than or equal to 60 hours, greater than or equal to 80 hours, greater than or equal to 100 hours, greater than or equal to 120 hours, greater than or equal to 140 hours, greater than or equal to 160 hours, greater than or equal to 180 hours, greater than or equal to 200 hours, or greater than or equal to 220 hours. In some embodiments, the step of exposing the polymeric material to the non-solvent of the polymeric material is performed for less than or equal to 240 hours, less than or equal to 220 hours, less than or equal to 200 hours, less than or equal to 180 hours, less than or equal to 160 hours, less than or equal to 140 hours, less than or equal to 120 hours, less than or equal to 100 hours, less than or equal to 80 hours, less than or equal to 60 hours, less than or equal to 50 hours, less than or equal to 40 hours, less than or equal to 30 hours, less than or equal to 20 hours, less than or equal to 15 hours, less than or equal to 10 hours, less than or equal to 8 hours, less than or equal to 6 hours, less than or equal to 4 hours, or less than or equal to 2 hours. Combinations of the above-referenced ranges are also possible (e.g., greater than or equal to 1 hour and less than or equal to 240 hours). Other ranges are also possible.

In some embodiments, the step of introducing to the polymeric material, a solution comprising a second water soluble polymer, different than the first water soluble polymer, and an optional osmotic agent (e.g., a salt) comprises heating the polymeric material and the solution to a temperature of greater than or equal to 25° C., greater than or equal to 30° C., greater than or equal to 35° C., greater than or equal to 40° C., greater than or equal to 45° C., greater than or equal to 50° C., greater than or equal to 55° C., or greater than or equal to 60° C. In some embodiments, the polymeric material and the solution are heated to a temperature less than or equal to 65° C., less than or equal to 60° C., less than or equal to 55° C., less than or equal to 50° C., less than or equal to 45° C., less than or equal to 40° C., less than or equal to 35° C., or less than or equal to 30° C. Combinations of the above-referenced ranges are also possible (e.g., greater than or equal to 25° C. and less than or equal to 65° C.). Other ranges are also possible.

In some cases, the solution may be flowed adjacent (e.g., directly adjacent) the polymeric material for a particular amount of time. In some embodiments, the solution is flowed adjacent the polymeric material for greater than or equal to 3 hours, greater than or equal to 5 hours, greater than or equal to 6 hours, greater than or equal to 8 hours, greater than or equal to 10 hours, greater than or equal to 12 hours, greater than or equal to 16 hours, greater than or equal to 20 hours, greater than or equal to 24 hours, greater than or equal to 28 hours, greater than or equal to 32 hours, greater than or equal to 36 hours, greater than or equal to 40 hours, or greater than or equal to 44 hours. In some embodiments, the solution is flowed adjacent the polymeric material for less than or equal to 48 hours, less than or equal to 44 hours, less than or equal to 40 hours, less than or equal to 36 hours, less than or equal to 32 hours, less than or equal to 28 hours, less than or equal to 24 hours, less than or equal to 20 hours, less than or equal to 16 hours, less than or equal to 12 hours, less than or equal to 10 hours, less than or equal to 8 hours, less than or equal to 6 hours, or less than or equal to 5 hours. Combinations of the above-referenced ranges are also possible (e.g., greater than or equal to 3 hours and less than or equal to 48 hours). Other ranges are also possible. Combinations of the above-referenced temperatures and times are also possible.

In some embodiments, the method comprises annealing the polymeric material to a temperature of greater than or equal to 80° C. (e.g., greater than or equal to 80° C. and less than or equal to 250° C.) for greater than or equal to 60 minutes (e.g., greater than or equal to 60 minutes and less than or equal to 480 minutes). In some embodiments, the polymeric material is annealed at a temperature of greater than or equal to 80° C., greater than or equal to 90° C., greater than or equal to 100° C., greater than or equal to 120° C., greater than or equal to 140° C., greater than or equal to 160° C., greater than or equal to 180° C., greater than or equal to 200° C., greater than or equal to 220° C., or greater than or equal to 240° C. In some embodiments, the polymeric material is annealed at a temperature of less than or equal to 250° C., less than or equal to 240° C., less than or equal to 220° C., less than or equal to 200° C., less than or equal to 180° C., less than or equal to 160° C., less than or equal to 140° C., less than or equal to 120° C., less than or equal to 100° C., or less than or equal to 90° C. Combinations of the above-referenced ranges are also possible (e.g., greater than or equal to 80° C. and less than or equal to 250° C.). Other ranges are also possible.

In some embodiments, the polymeric material is annealed for greater than or equal to 30 minutes, greater than or equal to 60 minutes, greater than or equal to 80 minutes, greater than or equal to 100 minutes, greater than or equal to 120 minutes, greater than or equal to 160 minutes, greater than or equal to 200 minutes, greater than or equal to 240 minutes, greater than or equal to 280 minutes, greater than or equal to 320 minutes, greater than or equal to 360 minutes, greater than or equal to 400 minutes, or greater than or equal to 440 minutes. In some embodiments, the polymeric material is annealed for less than or equal to 480 minutes, less than or equal to 440 minutes, less than or equal to 400 minutes, less than or equal to 360 minutes, less than or equal to 320 minutes, less than or equal to 280 minutes, less than or equal to 240 minutes, less than or equal to 200 minutes, less than or equal to 160 minutes, less than or equal to 120 minutes, less than or equal to 100 minutes, or less than or equal to 80 minutes. Combinations of the above-referenced ranges are also possible (e.g., greater than or equal to 60 minutes and less than or equal to 480 minutes). Other ranges are also possible. Combinations of the above-referenced temperatures and times are also possible.

In some embodiments, the core material may be air, water, a non-solvent liquid, a solid, or a gas. In some cases, the core material may be removed after formation of the polymeric material on the core material. The core material may be physically removed and/or dissolved, in some cases.

In an exemplary embodiment, the method comprises, with a mixture (e.g., a solution as described above and herein) comprising at least one water soluble polymer, a salt, and water, wherein the at least one water soluble polymer is present in the mixture in an amount greater than or equal to 13 w/w% versus the total weight of the mixture, performing the steps of: heating the mixture to a temperature greater than or equal to 65° C., after heating the mixture, cooling the mixture to a temperature at least 20° C. cooler than a melting point of the mixture and mechanically shaping the mixture. In some embodiments, after cooling the mixture, the mixture may be extruded at a temperature greater than or equal to 65° C. on a core material to form the polymeric material disposed on the core material. The method may involve exposing the polymeric material to non-solvent of the polymeric material at a temperature less than or equal to 28° C. for greater than or equal to 4 hours and removing at least a portion of the core material from the polymeric material.

In some embodiments, the step of cooling the mixture comprises cooling to a temperature at least 20° C., at least 25° C., at least 30° C., at least 35° C., at least 40° C., at least 45° C., at least 50° C., at least 60° C., at least 70° C., at least 80° C., or at least 90° C. cooler than a melting point of the mixture. In some embodiments, the step of cooling the mixture comprises cooling to a temperature of less than or equal to 100° C., less than or equal to 90° C., less than or equal to 80° C., less than or equal to 70° C., less than or equal to 60°C., less than or equal to 50° C., less than or equal to 45° C., less than or equal to 40° C., less than or equal to 35° C., less than or equal to 30° C., or less than or equal to 25° C. lower than a melting point of the mixture. Combinations of the above-referenced ranges are also possible (e.g., at least 20° C. and less than or equal to 100° C. lower). Other ranges are also possible. The mixture may be cooled for any suitable amount of time.

In some embodiments, the mixture may be mechanically shaped. In some embodiments, the composition (e.g., prior to extrusion i.e. the mixture) may be mechanically shaped by kneading, rolling, cutting, and combinations thereof.

In some embodiments, the mixture is mixed at a temperature of greater than or equal to 80° C., greater than or equal to 90° C., greater than or equal to 100° C., greater than or equal to 120° C., greater than or equal to 140° C., greater than or equal to 160° C., greater than or equal to 180° C., greater than or equal to 200° C., greater than or equal to 220° C., or greater than or equal to 240° C. In some embodiments, the mixture is mixed at a temperature of less than or equal to 250° C., less than or equal to 240° C., less than or equal to 220° C., less than or equal to 200° C., less than or equal to 180° C., less than or equal to 160° C., less than or equal to 140° C., less than or equal to 120° C., less than or equal to 100° C., or less than or equal to 90° C. Combinations of the above-referenced ranges are also possible (e.g., greater than or equal to 80° C. and less than or equal to 250° C.). Other ranges are also possible.

In some embodiments, the method comprises sorption of a second water-soluble polymer into the polymeric material, as described above and herein.

Any of a variety of methods may be used to produce spatial variation in one or more physical properties of a polymeric material (e.g., to form a plurality of portions of a polymeric tube having different properties). According to some embodiments, variation in physical properties is induced by spatially varying annealing of the polymeric material, such that some portions of the polymeric material are more annealed than others. Annealing may be associated with changes in physical properties of polymers. For example, without wishing to be bound by any particular theory, annealing may facilitate evolution of a polymeric microstructure towards a thermodynamic equilibrium, e.g., by accelerating thermodynamically favorable changes in the crystallinity, anisotropy, interfacial structure and/or pore structure of the polymeric material.

Annealing may be spatially controlled in any of a variety of suitable ways. For example, in some embodiments, a method comprises inducing a thermal gradient in a polymeric material (e.g., a polymeric tube) such that thermal annealing may be performed at a differential rate across the volume of the polymeric material. Thermal annealing may be performed in any of a variety of suitable ways. For example, in some embodiments, a polymeric tube is annealed using selective or gradient temperatures. In some embodiments, the gradient can be generated by convective heating elements directed at a portion of the extruded segment. For example, the heating elements can be directed at a first portion of a polymeric tube but not at a second portion of the polymeric tube, such that annealing of the second portion of the polymeric tube occurs principally via thermal transport from the first portion of the polymeric tube. In some embodiments, a polymeric tube be placed in an oven such that a portion of the extruded segment falls outside of the oven, for example. Such configurations may be advantageous for establishing a gradient in physical properties along the length of the lumen, for example. As another example, in some embodiments a fluid may be transported through the polymeric tube during heating of the polymeric tube, to produce a thermal gradient perpendicular to the lumen. For example, a tube may be exposed to the uniform external heat of an oven while a fluid throws through the tube, such that a portion of the polymeric material closer to the lumen is cooler than a portion of the polymeric material further from the lumen. Likewise, in some embodiments a heated fluid may be passed through the polymeric tube to heat a portion closest to the lumen relative to portions further from the lumen.

In some embodiments, spatially varying annealing does not require establishing a temperature gradient. For example, in some embodiments, the method comprises introducing a gradient by dipping the polymeric tube into a thermal reservoir (e.g., a reservoir containing a fluid such as oil, air, an inert gas such as argon or nitrogen, or any of a variety of other suitable fluids) maintained at a fixed temperature such that different portions of the tube are exposed to the reservoir for different durations of time. The thermal reservoir may contain a fluid that does not dissolve the polymeric tube. In some embodiments, the thermal reservoir contains a fluid that does not wet the polymeric tube (e.g., a hydrophobic liquid or a gas). The polymeric tube may be exposed to (e.g., dipped into) the thermal reservoir for different durations at different spatial positions of the tube to achieve differential annealing effects, in some embodiments.

The thermal reservoir may have any of a variety of suitable temperatures. In some embodiments, a suitable thermal reservoir has a temperature of greater than or equal to 50° C., greater than or equal to 60° C., greater than or equal to 70° C., greater than or equal to 80° C., greater than or equal to 90° C., greater than or equal to 100° C., greater than or equal to 110° C., greater than or equal to 120° C., greater than or equal to 130° C., greater than or equal to 140° C., greater than or equal to 150° C., or greater than or equal to 160° C. In some embodiments, a suitable thermal reservoir has a temperature of less than or equal to 170° C., less than or equal to 160° C., less than or equal to 150° C., less than or equal to 140° C., less than or equal to 130° C., less than or equal to 120° C., less than or equal to 110° C., less than or equal to 100° C., less than or equal to 90° C., less than or equal to 80° C., less than or equal to 70° C., or less than or equal to 60° C. Combinations of these ranges are also possible (e.g., greater than or equal to 50° C. and less than or equal to 170° C., or greater than or equal to 80° C. and less than or equal to 150° C.). Other ranges, both higher and lower than those described above, are also possible, as the disclosure is not so limited.

To provide yet another approach for producing differential annealing in a polymeric material, in some embodiments a polymeric material (e.g., a polymeric tube) may be completely annealed (e.g., such that the entire tube reaches an equilibrium), and then a portion of the polymeric material (e.g., a portion of a polymeric tube) may be selectively heated and cooled rapidly, e.g., to remove it from equilibrium without perturbing the equilibrium of other portions of the polymeric tube.

In some embodiments, the extruded segment is annealed using selective or gradient solvent exposure and/or extraction of solvent components (e.g. salts, additives, secondary hydrophilic polymer, and the like). The selective or gradient exposure and/or extraction can be configured to provide differential physical properties (e.g. Young's elastic modulus) along the length of the extruded segment. For example, a portion of the polymeric tube along the length of its lumen may be selectively exposed to a solvent or to solvent extraction conditions. Similarly, the selective or gradient exposure and/or extraction can be configured to provide differential physical properties along a transverse dimension of the polymeric tube. For example, a solvent or solvent extraction conditions may be passed through the interior of a polymeric tube, or else the ends of the lumen may be capped to selectively expose only the exterior surface of a polymeric tube to a solvent or to solvent extraction conditions. As another example, the polymeric tube can be exposed to the solvent or solvent extraction conditions for different durations at different positions of the polymeric tube. For example, the polymeric tube can be dipped into the solvent or solvent extraction conditions at a rate that results in differential total dip durations along the length of the polymeric tube.

And, according to some embodiments, it is possible to introduce spatial variation in physical properties of a polymeric material (e.g., a polymeric tube) without annealing. For example, in some embodiments, differential physical properties may be induced, e.g., by differential cross-linking. For example, portions of polymeric material may be cross-linked to different extents by selective exposure of one or more portions of the polymeric material to a crosslinking agent, or to another driving force for cross-linking (e.g., by exposing the polymeric material to light with a spatially varying intensity, in the case of photo −crosslinking, or to differential heating or solvent conditions that could affect the activity of a cross-linking agent).

It should, of course, be understood that more than one of the foregoing methods may be employed, alone or in combination, to produce one or more spatial gradients in properties of a polymeric material (e.g., a polymeric tube). It should further be understood that other methods of producing spatial variation in polymeric properties are also possible, as the disclosure is not limited to the specific methods described above.

In some embodiments, the polymeric materials and/or polymeric tubes described herein may be exposed to and/or comprise a humectant. For example, in some embodiments, polymeric tube 10 comprises humectant 70, as shown illustratively in FIG. 5G. In some embodiments, at least a portion of the humectant is disposed on a surface (e.g., an inner lumen and/or an abluminal surface) of the polymeric material and/or polymeric tube (e.g., the body portion). For example, in some embodiments, a portion of humectant 70 is disposed on a surface of polymeric tube 10. In some embodiments, greater than or equal to 30%, greater than or equal to 40%, greater than or equal to 50%, greater than or equal to 60%, greater than or equal to 70%, greater than or equal to 80%, greater than or equal to 90%, or all of the humectant is disposed on a surface of the polymeric material and/or polymeric tube (e.g., the body portion). In some embodiments, less than or equal to 100%, less than or equal to 90%, less than or equal to 80%, less than or equal to 70%, less than or equal to 60%, less than or equal to 50%, less than or equal to 40% of the humectant is disposed on a surface of the polymeric material and/or polymeric tube (e.g., the body portion). Combinations of these ranges are also possible (e.g., 40-100%).

In some embodiments, at least a portion of the humectant is inside the polymeric material and/or polymeric tube (e.g., the body portion). In some embodiments, at least a portion of the humectant is inside the polymeric material and/or polymeric tube (e.g., the body portion). For example, in some embodiments, a portion of humectant 70 is inside polymeric tube 10 (e.g., absorbed into the bulk of the polymeric tube). In some embodiments, greater than or equal to 30%, greater than or equal to 40%, greater than or equal to 50%, greater than or equal to 60%, greater than or equal to 70%, greater than or equal to 80%, greater than or equal to 90%, or all of the humectant is inside the polymeric material and/or polymeric tube (e.g., the body portion). In some embodiments, less than or equal to 100%, less than or equal to 90%, less than or equal to 80%, less than or equal to 70%, less than or equal to 60%, less than or equal to 50%, less than or equal to 40% of the humectant is inside the polymeric material and/or polymeric tube (e.g., the body portion). Combinations of these ranges are also possible (e.g., 30-100%).

In some embodiments, the humectant is a non-ionic surfactant (i.e. a surfactant having an uncharged hydrophilic head and a hydrophobic tail) or a zwitterionic surfactant (i.e. a surfactant having a net uncharged hydrophilic head and a hydrophobic tail). In some embodiments, the humectant is a non-ionic surfactant selected from the group consisting of sugar alcohols, poloxamer, triacetin, a-hydroxy acids, polyethylene glycol, polypropylene glycol, ethylene glycol, propylene glycol, hexylene glycol, butylene glycol, glycerol, sorbitol, mannitol, xylitol, maltitol, erythritol, threitol, arabitol, ribitol, galactitol, fucitol, iditol, inositol, volemitol, malitol, lactitol, maltotriitol, maltotetraitol, polyglycitols, and combinations thereof. In some embodiments, the humectant comprises an oil such as vitamin E. In some embodiments, the humectant comprises a salt such as sodium chloride, potassium chloride, and/or phosphocholine.

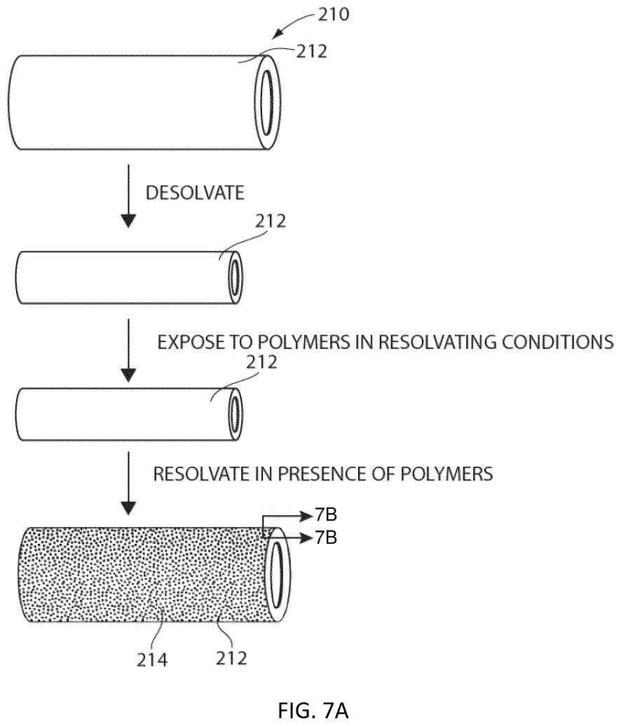

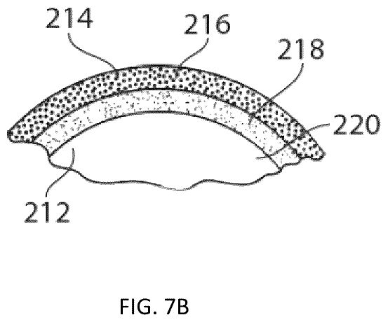

In some embodiments, the polymeric materials and/or devices described herein are exposed to and/or comprise greater than or equal to 0.1 w/w% humectant, greater than or equal to 0.5 w/w% humectant, greater than or equal to 1 w/w% humectant, greater than or equal to 5 w/w% humectant, greater than or equal to 10 w/w% humectant, or greater than or equal to 20 w/w% humectant. In some embodiments, the polymeric materials and/or devices described herein are exposed to and/or comprise less than or equal to 30 w/w% humectant, less than or equal to 25 w/w% humectant, less than or equal to 20 w/w% humectant, less than or equal to 15 w/w% humectant, less than or equal to 10 w/w% humectant, less than or equal to 5 w/w% humectant, or less than or equal to 1 w/w% humectant. Combinations of these ranges are also possible (e.g., 0.1-30 w/w% humectant or 1-10 w/w% humectant). A porous solid (e.g., made by the apparatus of FIGS. 5D-5F) may be annealed. Further, a porous solid, with or without prior annealing, may be processed to further include bulk incorporated polymers. In FIG. 7A, material 210 comprising porous solid matrix 212 is desolvated, exposed to a mixture comprising polymers that are in a resolvating solvent, and resolvated in the mixture to form material 212 with bulk incorporated polymers 214. A cross section of matrix 212 (FIG. 7B) reveals an outermost zone 216 wherein pores of matrix 212 are filled, an intermediate zone 218 wherein there is a lesser density of polymers in the pores, with less filling and/or fewer of the pores being occupied, and an inner zone 220 wherein polymers have not penetrated. The matrix can be solvated and/or desolvated prior to exposure to the mixture, provided that it is desolvated when exposed to the mixture so that water soluble polymers can be moved into the matrix.

In some embodiments, a method for humectifying a device and/or polymeric material comprises placing an extruded segment into a solution comprising the humectant (e.g., glycerol or poloxamer). In some embodiments the solution comprises greater than or equal to 1 w/w%, greater than or equal to 5 w/w%, greater than or equal to 10 w/w%, greater than or equal to 15 w/w%, greater than or equal to 20 w/w%, or greater than or equal to 25 w/w% humectant. In some embodiments, the solution comprises less than or equal to 35 w/w%, less than or equal to 30 w/w%, less than or equal to 25 w/w%, less than or equal to 20 w/w%, less than or equal to 15 w/w%, less than or equal to 10 w/w%, or less than or equal to 5 w/w% humectant. Combinations of these ranges are also possible (e.g., 1-35 w/w%). In some embodiments, the solution comprises a surfactant. In some embodiments, the solution comprises PBS.

In some embodiments, the extruded segment is placed in the solution for a period of time. In some embodiments, the period of time is greater than or equal to 1 hour, greater than or equal to 2 hours, or greater than or equal to 3 hours. In some embodiments, the period of time is less than or equal to 4 hours, less than or equal to 3 hours, or less than or equal to 2 hours. Combinations of these ranges are also possible (e.g., 3 hours, or 1-4 hours).

In some embodiments, the solution is maintained at a temperature during exposure of the extruded segment to the solution. In some embodiments, the temperature is greater than or equal to 20° C., greater than or equal to 30° C., greater than or equal to 37° C., greater than or equal to 40° C., greater than or equal to 50° C., or greater than or equal to 60° C. In some embodiments, the temperature is less than or equal to 70° C., less than or equal to 60° C., less than or equal to 55° C., less than or equal to 50° C., less than or equal to 40° C., less than or equal to 37° C., or less than or equal to 30° C. Combinations of these ranges are also possible (e.g., 20-70° C., 37-55° C., or 45° C.).

In some embodiments, after the extruded segment is removed from the solution, the extruded segment can be dried (e.g., in a convection oven). In some embodiments, the extruded segment is dried at a certain temperature. In some embodiments, the temperature is greater than or equal to greater than or equal to 20° C., greater than or equal to 30° C., or greater than or equal to 40° C. In some embodiments, the temperature is less than or equal to 50° C., less than or equal to 40° C., or less than or equal to 30° C. Combinations of these ranges are also possible (e.g., 30° C., or 20-50° C.). In some embodiments, the extruded segment is dried for a period of time. In some embodiments, the period of time may be greater than or equal to 1 hour, greater than or equal to 2hours, or greater than or equal to 3 hours. In some embodiments, the period of time may be less than or equal to 4 hours, less than or equal to 3 hours, or less than or equal to 2hours. Combinations of these ranges are also possible (e.g., 3 hours, or 1-4 hours).

A biologically active agent may be incorporated into the devices and/or devices described herein using any suitable method. For example, in some embodiments, the first water soluble polymer may be mixed with water. In some embodiments, the biologically active agent may be suspended or solubilized in water prior to solution compounding. The biologically active agent may be micronized, aggregated, and/or untreated, in some cases, when combined into the solution comprising the water-soluble polymer and water. In some embodiments, the biologically active agent may be mixed with the water-soluble polymer and water prior to heating the solution as described herein. In some embodiments, the biologically active agent may be added as the temperature is lowered upon cooling after bulk incorporation of polymers as described herein.

In some embodiments, to solubilize or suspend the active agent in the water, the system may include co-solvents e.g., that have a boiling point higher than the solubilization temperature of the compounding mixture such as N, N-dimethylformamide, suspension agents such as ionic or non-ionic surfactants, oils and castor oil. If the active agent is insoluble, in some cases, the biologically active agent may be micronized and/or made into nanoparticles. The biologically active agent may be mixed, in some cases, into the melt mixture as described herein and e.g., placed under high shear to solubilize.

In some embodiments, the biologically active agent may be incorporated into the body portion via sorption of the biologically active agent. In an exemplary embodiment, the water-soluble polymer material is shaped into near final dimensions through a forming process (e.g., electrospinning, electrospraying, melt spinning, wet spinning, extrusion, molding, casting, coating, and/or non-solvent entrainment). In some embodiments, the water-soluble biologically active agent may then be sprayed, absorbed, or adsorbed into and/or onto the polymer (e.g., PVA) matrix as a solution. The sorption process may occur, in some cases, post-shape forming, post-annealing, post-crosslinking, post-sterilization, or in situ prior to device placement in the subject.

In some embodiments, the biologically active agent solution may be further modified to increase solubility (e.g., by modulating pH and/or temperature, by adding an osmotic agent or cosolvent). In some embodiments, hydrolysable bonds (esters and amides) are used to bind active agents or active agent complexes into the polymer material.

In some embodiments, the biologically active agent may be encapsulated. For example, in some embodiments, the biologically active agent may be incorporated into (e.g., mixed) the third water soluble polymer described above and herein.

In an exemplary embodiment, the biologically active agent is added into the compounding mixture during solubilization. Biologically active agent/water soluble polymer mixture may be, in some cases, dehydrated and physically crosslinked e.g., at temperatures above 120° C. Without wishing to be bound by theory, in some embodiments, after cross-linking, the material may be brittle and is micronized, lyophilized, and/or sieved into a powder with a maximum particle size of e.g., 50 microns. In some embodiments, the powder is incorporated into the shape forming process at a mixing or solubilization stage. In some embodiments, the third water-soluble polymer comprises PVA. In some embodiments, the bulk PVA used for the initial encapsulation may contain a PVA with a higher molecular weight than that of the bulk porous PVA (e.g., the first water soluble polymer, the second water soluble polymer). The bulk porous PVA containing the micronized powder may be, in some cases, physically crosslinked e.g., at temperatures over 120° C. Advantageously, and without wishing to be bound by theory, the encapsulation and micronization of the biologically active agent may increase the release rate as compared to the release rate of a biologically active agent without encapsulation or micronization.

In some embodiments, cross-linking of the third water soluble polymer can be achieved by UV-crosslinking, chemical cross-linking (e.g., glutaraldehyde, bis(hydroxyethl) sulfone, maleic acid, etc.), and/or radiation cross-linking (e.g., gamma) prior to micronization. In some embodiments, traditional encapsulation methods may be used to micronize to less than 50 microns and/or extend a controlled release from a microparticle or nanoparticle e.g., through in situ oil in water emulsion or water in oil emulsion or cavity molding.

In some embodiments, the particles comprising the biologically active agent may be produced in situ with fully polymerized polymer, prepolymer with a crosslinker or initiator, monomer and initiator, or two or more monomers that self-polymerize, or combinations thereof.

As described herein, in some embodiments, the biologically active agent may be present within the plurality of pores of the body portion of the device (e.g., FIGS. 5B-5C). In some such embodiments, the biologically active agent may be released upon e.g., hydration and/or expansion/elongation of the device. Incorporation of the biologically active agent into the plurality of pores may use any suitable method. For example, in some embodiments, the biologically active agent may be mixed with a second water soluble polymer as described herein, such that the second water soluble polymer and biologically active agent are disposed within the plurality of pores. In some embodiments, the biologically active agent may be adsorbed/absorbed into the plurality of pores.

In some embodiments, the biologically active agent may be solubilized and infused into the body portion via a channel of the device (e.g., lumen 25 of FIGS. 5A-5C). Such devices may be useful as delayed release (e.g., long-term release) and/or reloadable devices.

In some embodiments, a biologically active agent with a water-soluble polymer is co-extruded as a center layer between an outer and inner layer containing a non-agent bulk polymer (e.g., PVA). In an exemplary embodiment, the biologically active agent is compatible with the bulk polymer (with and will adhere well, without delamination. In some embodiments, the biologically active agent layer is distant from the surface allowing for a bindable polymer to be adsorbed and absorbed into those surface layers.

In another exemplary embodiment, an agent binding complex can also be added, such as a counterionic system, where an anionic biologically active agent will bind to upon absorption. Without wishing to be bound by theory, upon swelling in an agent-soluble solution, the biologically active agent may migrate into the matrix and bind with a center layer of the device. The outer/inner later may be washed, in some cases, with more rigor than without this center later. In yet another exemplary embodiment, the biologically active agent-containing layer is on one or more surfaces or compatible with drug complex (e.g., allowing for a bindable polymer to be adsorbed and absorbed through the bulk).

Any of a variety of processes may be used for making a porous solid including bulk incorporated polymers. In some embodiments, a radiopaque (RO) agent is included in an extrusion process. According to some embodiments, post-processing is included in an extrusion process after drying the extrudate on steel mandrels.

Artisans reading this disclosure will be able to adapt its principles in light of what is known about extrusion or other forming arts to make alternative processes and devices that achieve the same end products as described herein. A scaled-up embodiment of this process may be adapted for use with, for example, a multi-zone screw extruder, with the solvent mixture provided via a suitable injector or a hopper and the zones controlled to provide a cold extrusion. Features such as the syringe pump can be replaced by a suitably metered and controlled liquid or solid polymer feed system.

Fukumori et al. (2013), Open J. Organic Polymer Materials 3:110-116 reported a freeze-thaw process of making poly(vinyl alcohol) (PVA) materials with a Young's modulus of 181 MPa with a Young's modulus of about 5 MP or more requiring at least about 3 cycles in the samples they tested. The process of making these gels required multiple freeze-thaw cycles. The resultant materials were tested in a dry condition and are not comparable to strengths measured at EWC. Fukumori et al. reported that the crystalline content of the materials increased with the number of freeze-thaw cycles and attributed the strength of the materials to large crystals being formed as the freeze-thaw cycles progressed, with the larger crystals forming superior crosslinks that increased the Tg of the materials. The nature of these processes produces a dried material. Moreover, as discussed below, a freeze-thaw process produces macropores.

In some embodiments, processes herein are free of freeze-thaw processes and/or free of a freezing process and/or free of a thawing process. Further the processes can be used to make solid porous materials that have little or no swelling, e.g., 0%-100% w/w swelling at EWC, even in an absence of covalent crosslinking agents Artisans will immediately appreciate that all ranges and values between the explicitly stated bounds are contemplated, with, e.g., any of the following being available as an upper or lower limit: 0, 5, 10, 15, 20, 25, 30, 40, 50, 60, 70, 80, 90, 95, 100 % w/w, with swelling measured as % swelling=100×(Total weight at EWC-dry weight)/dry weight, with the dry weight being the weight of the material without water.

In some embodiments, the extruded samples have a horizontal chain orientation and alignment along the length of samples (in direction of extrusion). A polymeric chain orientation produced by the extrusion process. Without wishing to be bound by theory, it is believed that this horizontal chain orientation and alignment along the length of the samples contributes to the inner diameter and/or outer diameter increasing by a larger percentage than the percentage increase in length when the samples swell, in some embodiments.

In some embodiments, it is useful to have a combination of one or more of: extrusion of a hydrophilic polymer in a solvent; a cold extrusion, and extrusion into a bath that quickly removes solvent from the extrudate. Further, in some embodiments, additional solvent-removing and/or annealing processes provide further utility for making desirable porous solids.

In some embodiments, requirements for a nanoporous material include high polymer concentrations of more than about 10% w/w in the polymer-solvent mixture with high levels of crosslinking. Artisans will immediately appreciate that all ranges and values between the explicitly stated bounds are contemplated, with, e.g., any of the following being available as an upper or lower limit: 10, 12, 15, 16, 17, 18, 19, 20, 25,30, 35, 40, 45, 50, 55, 60, 70, 80, 90, 95, 99% w/w of the polymer in the total weight of the polymer-solvent mixture. In some embodiments, the polymer is to be substantially solvated, meaning it is a true solution or at least half the polymer is dissolved and the rest is at least suspended. In some embodiments, the solvation of the polymer contributes to the alignment of the polymer chains in an extrusion and to crosslinking among the polymers. Without being bound to a particular theory, it is likely that high concentration of the starting polymer-solvent mixture can help with this. And the probable chain alignment of the material as it passes through a die, according to some embodiments, is thought to promote more intrapolymer versus interpolymer crosslinking. An extrudate or an otherwise formed mixture entering a desolvating environment, whether gas or liquid, is thought to further collapse pore structure before the densely concentrated polymer has completely crosslinked, in some embodiments, thereby improving chain proximity and promoting additional crosslink density. Depositing the extruded or otherwise formed material directly into a solvent removing environment is helpful in some embodiments. In some embodiments, further solvent-removal can be continued to collapse the material until reaching a desired end point in structure and/or properties. An annealing process can further contribute to strength in some embodiments.

Frozen methods, on the other hand, rely on increased strengthening by forcing super-concentrated microregions to also achieve chain proximity and improve crosslink density, but retain a macro porosity due to the presence of ice crystals in the total gel structure. Desolvation creates forced super-concentrated microregions but these do not create macropores. In contrast, a pre-established gel prior to a dehydration or freezing is by nature of that process formed with macropores. Further, the work of the inventors indicates that such nanoporous solids have greater strength than macroporous materials.

Hydrogels can also be made by using a lower polymer concentration in the polymer-solvent mixture, generally less than 10% w/w of polymer in the polymer-solvent mixture. Artisans will immediately appreciate that all ranges and values between the explicitly stated bounds are contemplated, with, e.g., any of the following being available as an upper or lower limit: 2, 5, 7, 8, 9, 10% w/w of the polymer in the total weight of the polymer-solvent mixture. Further, or alternatively, the polymer-solvent mixture is not extruded into a solvent removing environment.

Microporous materials may be made with process conditions intermediate to nanoporous solids and hydrogels. One embodiment is to prepare a material using conditions comparable to making a nanoporous material but to stop solvent removal before solvent removal reaches a nanoporous solid structure.

Extrusion of hydrophilic polymers in a solvent is helpful to make high strength materials. Use of a solvent in an extrusion starting material is, at the least, uncommon. Typically, an extrusion uses a solid material that has been heated to a flowable temperature and then extruded, and later cooled by a variety of methods. For instance, it is believed that thermoplastic extrusion of a pure PVA is possible. But such an extrusion would lack the polymeric structure that is needed to make porous solids and would instead exhibit properties more similar to a conventional thermoplastic material. According to a theory of operation, a pure PVA extrusion would lack the quality of hydrogen bonding that takes place in an aqueous ionic solvent state. A temperature suitable for preparing the PVA to be flowable in an extrusion would create a poorly cohesive material at the die head so that a continuous shape does not form. It was difficult to make extruded PVAs to form high aspect shapes, e.g., tubes, and to use them in an extrusion process. Viscosities of PVA and other hydrophilic polymers are high, and difficult to get into solution. It was observed that a narrow working band of temperature was particularly useful, e.g., 85-95° C. Below about 85° C., PVA failed to truly melt, and thus did not become completely amorphous for extrusion. Above about 95° C., losses to boiling and evaporation made the process ineffective. These temperature ranges could be offset by increasing pressure above atmospheric, but a pressurized system is challenging to use and to scale. The processes are usefully performed at a temperature below a boiling point of the polymer-solvent materials.

The cohesive strength of the flowing polymer-solvent mixture was weak when exiting the die. The use of a core to support the mixture at the die is useful to hold the shape at the die. This condition is in contrast to a typical core extrusion used as a coating process, e.g., for coating wires for a mobile telephone charger. A typical process that avoids use of a solvent or a significant solvent concentration has a relatively higher cohesive strength that it exits the die that is readily capable of holding a tube, and do not relying on active bonding such as the hydrogen bonding in hydrophilic polymers that form the solid material in a coherent shape as it moves out of the die.

Passing the formed polymer-solvent mixture into solvent removal environment was useful. Most extrusions do not use bath temperatures at or below room temperature. Moreover, the use of a solvent removing bath is atypical relative to conventional processes the bath or other solvent removing environment helps solidify the extruded material sufficiently that it remains stable and concentric on the core, otherwise the melt would run into a tear drop shape. It would also be destroyed in the attempt to collect it at the end of the extrusion as it would still be molten. Conventional baths containing water would cause the PVA or similar hydrophilic polymer material to lose shape due to swelling, dissolution, or both. Molding processes that involve preparation of a polymer-solvent mixture that is formed in a mold and then processed into a solvent-removing environment do not have the advantages of alignment of chains observed in an extrusion. However, a suitably controlled temperature and solvent removal can yield materials with a high strength and controlled pore structure.

The porous solids are highly lubricious and can be used in a hydrated state and can be conveniently bonded to other materials. In the case of a catheter, for instance, extensions, luer locks, suture wings, and the like are useful. In some embodiments, copolymer extrusion is useful in ranges of the second polymer from 0.1% to 10% w/w or no more than 10% w/w of the first polymer, with no more than 5% w/w also being useful. Artisans will immediately appreciate that all ranges and values between the explicitly stated bounds are contemplated, with, e.g., any of the following being available as an upper or lower limit: 0.1, 0.2, 0.4, 0.5, 0.8, 1, 2, 3, 4, 5, 6, 8, 10% w/w.

In some embodiments, salts are useful to manipulate the strength of the materials. Without being limited to a particular theory, it is likely the salts are part of the physical crosslinking, in effect acting as small molecular weight crosslinkers between the polymer chains.

Some embodiments for polymer blends include at least one first hydrophilic polymer and at least one second hydrophilic polymer in a solvent that is extruded as described herein. Examples include combinations of one or more of poly(vinyl alcohol) (PVA), poly(vinyl acetate) (PVAc), poly(butyl alcohol) (PBA), PAA, PEG, PVP, polyalkylene glycols, a hydrophilic polymer, and combinations thereof. Examples of concentrations include the at least one second hydrophilic polymer being present at 1 part to 10,000 parts of the first hydrophilic polymer. Artisans will immediately appreciate that all ranges and values between the explicitly stated bounds are contemplated, with, e.g., any of the following being available as an upper or lower limit: 1, 2, 10, 100, 1000, 1500, 2000, 2500, 3000, 4000, 5000, 6000, 7000, 8000, 9000, 10000 parts. Examples of concentrations of polymers in a polymer-solvent mixture include a first polymer present at a first concentration and one or more further polymers present at a second concentration, with the first polymer concentration and the further polymer concentration being independently selected from 0.1-99%, e.g., 0.1, 0.2, 0.3, 0.4, 0.5, 0.6, 1, 2, 3, 4, 5, 6, 7, 8, 9, 10, 11, 12, 13, 14, 15, 16, 17, 18, 19, 20, 25, 30, 33, 35, 40, 45, 50 55, 60, 65 ,70, 75,80, 85, 90, 95% w/w. Further, non-hydrophilic polymers and/or non-hydrophilic blocks in block polymers, may be present, with concentrations of such polymers and/or such blocks generally being less than about 10% w/w, e.g., 0.1, 0.2, 1, 2, 3, 4, 5, 6, 7, 8, 9, 10 % w/w.

Some embodiments include porous matrices conditioned with water soluble polymers that lose no more than 20-90% w/w of the water-soluble polymer under comparable conditions; Artisans will immediately appreciate that all ranges and values between the explicitly stated bounds are contemplated, e.g., 20, 25, 30, 33, 40, 50, 60, 70, 80, 90% w/w.

In some embodiments, bulk incorporated materials may present a monolayer at the surface. The term monolayer means a layer that is a single molecule thick. The monolayer does not rely on cohesion between the molecules of the monolayer to remain stably present at the surface. At least one water soluble polymer forms the monolayer. In contrast, even a thin polymer coating that is cross-linked to itself has a thickness corresponding to the thickness of the network formed by the cross-linked polymers. For example, it may be possible to create a cross-linked PVA coating on a surface but such a coating relies on interconnections between molecules of the PVA and necessarily forms a crosslinked network. Accordingly, embodiments include a water-soluble polymer present on a surface of a porous solid without covalent bonding to the surface and without the polymer being part of a network.

In some embodiments, the bulk incorporated polymers are durably incorporated. In contrast, a layer of water soluble materials merely adsorbed to an underlying material, e.g., applied by dip coating or spraying, can be essentially removed from a hydrophilic substrate in most or all circumstances meaning at least 90% w/w of the materials can be separated from the underlying material in aqueous solution, e.g., 90° C. for 24 hours in physiological saline. Covalently bonded materials will not be removed under these conditions and some physically crosslinked networks of water-soluble polymers might not be removed but such networks are not preferable compared to a bulk incorporated polymer; for instance, they would likely be more thrombogenic or less durable. Covalent bonding involves use of chemically reactive moieties that can be avoided by bulk incorporation processes.

In some embodiments, it may be advantageous to coat a polymeric tube or other article, e.g., to reduce its dehydration and/or reduce its stiffness prior to implantation. The coating may comprise adding a water-soluble polymer (e.g., a fourth water soluble polymer as discussed above) as a coating of the polymeric material (e.g., the polymeric tube). Any of a variety of suitable methods may be used to apply the coating, including but not limited to dip coating, injection molding, casting, and/or welding (e.g., via ultrasound or radio frequency welding) of the coating to one or more other water soluble polymers. In some embodiments, the coating is a luminal coating (e.g., which may be applied by pumping a fluid through the lumen of a polymeric tube). In some embodiments of luminal coating, a fluid (e.g., pressurized air) may be flowed through the lumen after application of the coating (e.g., to remove excess materials).

It may be advantageous to pre-treat a polymeric material prior to application of a coating, depending on the embodiment. For example, the polymeric material may be treated to alter its hydration state, salt content, and/or temperature. In some embodiments, it may be advantageous to pre-treat the surface of the polymeric material with water, e.g., by wiping the surface of the polymeric material with water, or by humidifying the polymeric material. Without wishing to be bound by any particular theory, pre-treatment with water may change the hydration state of the polymeric material, thereby improving integration of the coating with the treated polymeric material. In some embodiments, the pre-treatment is used to change the temperature (e.g., by treating with heated water) in order to accelerate hydration or other treatment processes.

Other pre-treatments may control the swell ratio of the polymeric material. For example, in some embodiments the polymeric material is soaked in a salt solution (e.g., an aqueous solution of sodium chloride) prior to coating. Without wishing to be bound by any particular theory, treatment with a salt solution may, according to some embodiments, help to control the swell ratio of the polymeric material, e.g., which may reduce internal strain and the corresponding threat of delamination of the coated polymer. The salt may be included in the salt solution at any of a variety of appropriate concentrations. In some embodiments, the salt is included in an amount of greater than or equal to 0.5 wt %, greater than or equal to 1 wt %, greater than or equal to 1.5 wt %, greater than or equal to 2 wt %, greater than or equal to 2.5 wt %, greater than or equal to 3 wt %, greater than or equal to 3.5 wt %, greater than or equal to 4 wt %, greater than or equal to 4.5 wt %, greater than or equal to 5 wt %, greater than or equal to 5.5 wt %, greater than or equal to 6 wt %, greater than or equal to 6.5 wt %, greater than or equal to 7 wt %, greater than or equal to 7.5 wt %, greater than or equal to 8 wt %, greater than or equal to 8.5 wt %, greater than or equal to 9 wt %, or greater than or equal to 9.5 wt %. In some embodiments, the salt is included in an amount of less than or equal to 10 wt %, less than or equal to 9.5 wt %, less than or equal to 9 wt %, less than or equal to 8.5 wt %, less than or equal to 8 wt %, less than or equal to 7.5 wt %, less than or equal to 7 wt %, less than or equal to 6.5 wt %, less than or equal to 6 wt %, less than or equal to 5.5 wt %, less than or equal to 5 wt %, less than or equal to 4.5 wt %, less than or equal to 4 wt %, less than or equal to 3.5 wt %, less than or equal to 3 wt %, less than or equal to 2.5 wt %, less than or equal to 2 wt %, less than or equal to 1.5 wt %, or less than or equal to 1 wt %. Combinations of these ranges are also possible (e.g., greater than or equal to 0.5 wt % and less than or equal to 10 wt %, or greater than or equal to 1 wt % and less than or equal to 10 wt %). Other ranges, both higher and lower than those described above, are also possible, as the disclosure is not so limited.

Coating may be applied to any of a variety of suitable lengths of a polymeric tube, depending on the embodiment. In some embodiments, a coating is applied to a length of greater than or equal to 1 cm, greater than or equal to 2 cm, greater than or equal to 5 cm, greater than or equal to 8 cm, greater than or equal to 10 cm, greater than or equal to 12 cm, greater than or equal to 15 cm, greater than or equal to 18 cm, greater than or equal to 20 cm, greater than or equal to 22 cm, greater than or equal to 25 cm, or greater than or equal to 28 cm. In some embodiments, a coating is applied to a length of less than or equal to 30 cm, less than or equal to 28 cm, less than or equal to 25 cm, less than or equal to 22 cm, less than or equal to 20 cm, less than or equal to 18 cm, less than or equal to 15 cm, less than or equal to 12 cm, less than or equal to 10 cm, less than or equal to 8 cm, less than or equal to 5 cm, or less than or equal to 2 cm. Combinations of these ranges are also possible (e.g., greater than or equal to 1 cm and less than or equal to 30 cm, greater than or equal to 1 cm and less than or equal to 5 cm, or greater than or equal to 20 cm and less than or equal to 30 cm). Other ranges, both higher and lower than those described above, are also possible, as the disclosure is not so limited.

The length over which the polymeric tube is coated may depend, in some embodiments, on the desired application for the polymeric tube. In some embodiments, the coating is applied to an end portion of the polymeric tube via dip coating or spray coating. For example, in some embodiments, a coating is applied to a distal portion of the polymeric tube (e.g., to improve grip of that portion of the polymeric tube). In some embodiments, a coating is applied to a proximal portion of the polymeric tube (e.g., to reduce slipperiness of a tip for insertion into a patient, for example, in a catheter that functions by puncturing to produce an orifice, such as certain ventricular catheters).

A water soluble polymer (e.g., a fourth water soluble polymer) may be coated from a polymer solution onto a polymeric tube, depending on the embodiment. In some embodiments, water soluble polymer is dip-coated by drawing the polymer tube through a solution (e.g., withdrawing the polymeric tube from the solution) at any of a variety of appropriate rates. In some embodiments, a polymer tube is drawn at a rate of greater than or equal to 0.1 cm/min, greater than or equal to 1 cm/min, greater than or equal to 10 cm/min, greater than or equal to 20 cm/min, greater than or equal to 40 cm/min, greater than or equal to 60 cm/min, greater than or equal to 80 cm/min, or greater than or equal to 100 cm/min. In some embodiments, a polymer tube is drawn at a rate of less than or equal to 120 cm/min, less than or equal to 100 cm/min, less than or equal to 80 cm/min, less than or equal to 60 cm/min, less than or equal to 40 cm/min, less than or equal to 20 cm/min, less than or equal to 10 cm/min, less than or equal to 1 cm/min, or less. Combinations of these ranges are also possible (e.g., greater than or equal to 0.1 cm/min and less than or equal to 120 cm/min, or greater than or equal to 20 cm/min and less than or equal to 100 cm/min). Other ranges, both higher and lower than those described above, are also possible, as the disclosure is not so limited.

A polymer solution may include any of a variety of solvents. A few non-limiting examples of suitable solvents that may be used include water, any of a variety of alcohols (e.g., methanol, ethanol, one or more isomers of propanol, one or more isomers of butanol, one or more isomers of pentanol, one or more isomers of hexanol, one or more isomers of heptanol, one or more isomers of octanol, one or more isomers of nonanol, one or more isomers of decanol, one or more isomers of undecanol, one or more isomers of dodecanol), acetone, methyl ethyl ketone, and combinations thereof. Water solvents may be particularly advantageous, in some embodiments, e.g., because of the biocompatibility of water.

A water soluble polymer (e.g., the fourth water soluble polymer) may be present in solution at any of a variety of suitable concentrations. In some embodiments, a water soluble polymer is present in solution in an amount of greater than or equal to 0.1 wt %, greater than or equal to 1 wt %, greater than or equal to 2 wt %, greater than or equal to 5 wt %, greater than or equal to 8 wt %, greater than or equal to 10 wt %, or greater than or equal to 12 wt %. In some embodiments, a water soluble polymer is present in solution in an amount of less than or equal to 15 wt %, less than or equal to 12 wt %, less than or equal to 10 wt %, less than or equal to 8 wt %, less than or equal to 5 wt %, less than or equal to 2 wt %, or less than or equal to 1 wt %. Combinations of these ranges are also possible (e.g., greater than or equal to 0.1 wt % and less than or equal to 15 wt %, or greater than or equal to 1 wt % and less than or equal to 10 wt %). Other ranges, both higher and lower than those described above, are also possible, as the disclosure is not so limited.

Any of a variety of solution temperatures may be used to apply the coating from a solution. In some embodiments, the solution has a temperature of greater than or equal to 0° C., greater than or equal to 5° C., greater than or equal to 10° C., greater than or equal to 15° C., greater than or equal to 20° C., greater than or equal to 25° C., greater than or equal to 30° C., greater than or equal to 35° C., greater than or equal to 40° C., greater than or equal to 45° C., greater than or equal to 50° C., greater than or equal to 55° C., greater than or equal to 60° C., greater than or equal to 65° C., greater than or equal to 70° C., greater than or equal to 75° C., greater than or equal to 80° C., greater than or equal to 85° C., greater than or equal to 90° C., or greater than or equal to 95° C. In some embodiments, the solution has a temperature of less than or equal to 100° C., less than or equal to 95°C., less than or equal to 90° C., less than or equal to 85° C., less than or equal to 80° C., less than or equal to 75° C., less than or equal to 70° C., less than or equal to 65° C., less than or equal to 60° C., less than or equal to 55° C., less than or equal to 50° C., less than or equal to 45° C., less than or equal to 40° C., less than or equal to 35° C., less than or equal to 30° C., less than or equal to 25° C., less than or equal to 20° C., less than or equal to 15° C., less than or equal to 10°C., or less than or equal to 5° C. Combinations of these ranges are also possible (e.g., greater than or equal to 0° C. and less than or equal to 100° C., greater than or equal to 0° C. and less than or equal to 25° C., or greater than or equal to 60° C. and less than or equal to 100° C.). Other ranges, both higher and lower than those described above, are also possible, as the disclosure is not so limited.

### Processing Systems and Parameters to Make Porous Materials

Processes are provided herein to create biocompatible porous solids such as microporous or nanoporous solid materials that possess low protein adsorption properties and provide a basis for non-biofouling devices. Modification of starting polymer concentration, molecular weight, solvent removal, forming processes, and hardening/annealing processes may be utilized to provide surface properties with reduced protein adsorption and other properties. Some embodiments include creation of various continuous shapes through extrusion of a polymeric mixture. The mixture may be further hardened and annealed. These processes may be used to create a tough and highly lubricious material. Embodiments include polymeric mixtures extruded into shapes possessing single or multiple lumens, of varied diameters and wall thickness.

An embodiment of a process for making a nanoporous solid material comprises heating a mixture that comprises a polymer and a solvent (a polymeric mixture), extruding the mixture into a solvent-removing environment, and removing the solvent from the crosslinked matrix until a nanoporous solid material is formed. One or more of these actions may be combined, depending on the process. Further, cooling the mixture as it passes out of the die is useful. Without being bound to a specific theory of operation, it appears that crosslinking the polymer during passage through the die initially forms a porous matrix that is not a true nanoporous solid material because, although it has spaces between polymer strands, it does not have a pore-structure. As the solvent is removed under appropriate conditions, the crosslinked structure becomes a nanoporous solid. The crosslinking starts when the polymeric mixture is extruded through a die, and as the mixture is cooled. The crosslinking may continue while the solvent is removed. The transition to form the nanoporous material takes place as the solvent is removed and, in general, is believed to be completed or essentially completed (meaning 90% or more) at this stage. The resultant material may be further processed by annealing with or without a presence of further solvents, or plasticizers. This process, and the other extrusion or other formation processes and/or materials set forth herein, including bulk incorporation processes, may be free of one or more of: covalent crosslinking agents, agents that promote covalent crosslinks, radiation that crosslinks polymer chains, freezing, thawing, freeze-thaw cycles, more than one freeze-thaw cycle, ice-crystal formation, foaming agents, surfactants, hydrophobic polymers, hydrophobic polymer segments, reinforcing materials, wires, braids, non-porous solids, and fibers.

The porous materials may be made by an extrusion process that comprises passing a polymeric mixture through a die into a cooling environment. The cooling environment may further be a solvent-removing environment. It is a dehydrating environment when the solvent is water. The die may have a core that passes through it so that the polymeric mixture may be formed around the core. Further solvent-removal environments and/or annealing environments may be used.

The extrusion process for a polymer-solvent mixture may be performed as a cold extrusion. The term cold extrusion refers to a process that involves passing a polymer-solvent mixture through a die and does not require heating the polymer-solvent mixture above its boiling point during the entire process of preparing the polymer-solvent mixture and extruding it. Accordingly, in a cold extrusion, the die head is kept below a boiling point of the polymer-solvent mixture. Although many solvents may be used, water is often a useful solvent in which case the die head is kept at 100° C. or less, although colder temperatures may be useful, as discussed above.

The term polymeric mixture refers to a polymer that is in solution, dissolved, or suspended in a solvent. A solvent may be, e.g., water, aqueous solution, an organic solvent, or combinations thereof. Heating the polymeric mixture may comprise heating the mixture to a temperature above the melting point of the polymer. In general, the solution transitions from a cloudy to a clear state when it reaches the melt point. An aqueous solution contains water, for instance from 10-100% (w/w or v/v) of the liquid being water; Artisans will immediately appreciate that all ranges and values between the explicitly stated bounds are contemplated, e.g., 10, 20, 30, 40, 50 60, 70, 80, or 90% or at least one of the same.

Extrusion is a useful process for forming the materials. Other forming processes may be used, for example, molding, casting, or thermal forming polymer-solvent mixtures. In general, a polymer-solvent mixture is prepared without boiling and formed into a shape that is exposed to solvent-removal conditions that are controlled to make a nanoporous or microporous material using the guidance provided herein. An annealing process may be included. Hydrogels that are not microporous or nanoporous materials can also be made.

The heated polymeric mixture may be molded or otherwise formed as it is cooled or molded/formed and immediately cooled. Formed is a broad term that refers to passing the material from an amorphous melted state into an end-user product or an intermediate shape for further processing. Forming encompasses casting, layering, coating, injection molding, drawing, and extrusion. The forming can be done using an injection molding set up, where the mold consists of a material with thermal conductive properties allowing it to be heated easily to enhance the flow of the injected polymeric mixture, and to be cooled rapidly in a cooling environment. In other embodiments, the molding process can be accomplished by extrusion of the polymeric mixture through a die to form continuous material.

Cooling the polymeric mixture may comprise, e.g., cooling an extruded material, as in the case of passing the polymeric material through a die. An embodiment for cooling is a liquid bath at a temperature at least 20° C. cooler than the polymeric mixture boiling point or alternatively below the polymeric mixture Tm, e.g., 20, 30, 40, 50, 60, 70, 80, 90, 100, 110° C. below the boiling point or polymeric Tm, or alternatively the bath or other environment being at a temperature from −50 to 30° C.; Artisans will immediately appreciate that all ranges and values between the explicitly stated bounds are contemplated, with, e.g., any of the following being available as an upper or lower limit: −50, −45, −25, −20, −10, −5, −4, 0, 15, 20, 25, 30° C. The cooling may be performed in a solvent removing environment. Freezing temperatures may be avoided. Without being bound to a particular theory of operation, the polymer chains are cooled to the point of promoting intermolecular hydrogen binding and immobilizing chain movement. This may occur at temperatures as high as 30° C., or higher if time is allowed. The bath may be aqueous, and, by adjustment with salt or other osmotic agents, may be provided at an osmotic value to perform solvent removal on aqueous materials that are at a relatively lower osmotic value through osmotic pressure and diffusion. The bath may also be other solvents that freeze at temperatures lower than water, so that temperatures below 0° C. may be used without freezing the solvent or materials. In the event that hydrophilic copolymers are used in conjunction with PVA, for instance, temperatures higher than 20° C. may be used as crosslinking and chain immobilization will occur at much higher temperatures.

A solvent-removing environment refers to an environment that significantly accelerates removal of a solvent as compared to drying at ambient conditions. Such an environment may be non-heating, meaning it is not above ambient temperature, e.g., not above 20° C. Such an environment may be a vacuum, e.g., a vacuum chamber, a salt bath, or a bath that removes the solvent in the polymeric mixture. For instance, an aqueous polymeric mixture may be introduced into an ethanol bath, with the ethanol replacing the water. The ethanol may subsequently be removed. A salt bath may be, e.g., a high salt concentration bath (1M to 6M). A time of processing in a solvent-removing environment and/or a cooling process may be independently chosen to be from 1 to 240 hours; Artisans will immediately appreciate that all ranges and values between the explicitly stated bounds are contemplated, with, e.g., any of the following being available as an upper or lower limit: 1, 2, 5, 10, 24 hours, 1, 2, 5, 7, 10 days. Salts may be salts that dissociate to make single, double, or triply charged ions.

One or a plurality of solvent-removing environments may be used, or one environment may be adjusted with respect to temperature. Thus, a cooling bath may be used followed by solvent removal in an oven or vacuum oven. A washing step may be performed before or after cooling or solvent removal, e.g., by soaking in a series of solvents of varying concentrations, varying salt solutions, varying proportions of ethanol or other solvents.

An embodiment is an extruded material that has been through a solvent-removal process comprising exposure to a salt bath, the material is soaked in a series of H₂O baths (new baths or exchanged) for a period of time (e.g., 2-48 hours, 4-24 hours) to remove excess salt from the cast material or end-user device. The material is removed from the wash step and dehydrated to remove excess water. Dehydration can be done using, e.g., temperatures ranging from 20-95° C. Dehydration is generally performed at 37° C. for greater than 24 hours.

An embodiment is a polymeric mixture that has been extruded or otherwise formed that is then exposed to a high salt concentration bath (1M to 6M) for an inversely correlated period of time; high salt reduces the time required for soaking; for instance, it is soaked for 16-24 hours in a 6M solution of NaCl. After soaking, the material is rinsed free of salt solution. The material is now toughened and can be removed from any mold pieces carried over from the initial formation. Alternatively, after a salt or other bath, the material is soaked in water baths and dehydrated to remove excess water. Dehydration can be done using temps ranging from 20-95° C. Dehydration may be performed at 37° C. for greater than 4 hours, greater than 24 hours, or in a range from 2 to 150 hours; Artisans will immediately appreciate that all ranges and values between the explicitly stated bounds are contemplated, with, e.g., any of the following being available as an upper or lower limit: 2, 4, 6, 8, 10, 12, 16, 24, 48, 72, 96, 120, 144, 150 hours. For instance, dehydration at 40° C. for 6-24 hours has been observed to be useful.

In another embodiment, NaCl is incorporated into the starting polymeric solution at concentrations ranging from 0.1 to 3M of the final polymeric mixture volume. A polymer is dissolved in a heated solution under agitation, then brought above its melt point. To this solution, dry NaCl is added slowly under agitation until completely dissolved. The slightly hazy solution is then drawn into a feed for the purpose of creating a shape, either through injection molding, casting, extrusion and/or drawing. A quench is performed at the end of each process to rapidly reduce the temperature and form a solid material. In this embodiment, no additional salt soak is required. After material hardening, if necessary, the material is removed from any molding process parts and rinsed in water to remove salt and dehydrated.

The term annealing, as used in the context of a semi-crystalline polymer or a solid porous material may refer to a heat treatment at an annealing temperature comparable to the melting temperature of the polymer or the polymers in the relevant material. This temperature is usually less than and is within about 0-15% of the melting temperature on an absolute temperature scale. Plasticizers or other additive materials may affect the melting temperature, usually by depressing it. For a pure PVA, for instance, the annealing temperature will be within about 10% of the melting point of the PVA; with other materials present, the annealing temperature will typically be lower. A theory of operation is that the annealing is a process that is a relaxation of stress combined with increase in the size of crystalline regions in the material being annealed. Unlike metals, annealing increases the strength of the annealed material. Annealing may be performed in one or more of: in air or in a gas or in an absence of oxygen or an absence of water, e.g., in nitrogen, in vacuum nitrogen, under argon, with oxygen scavengers, and so forth. For example, experiments have been made with annealing dehydrated PVA nanoporous materials. Annealing is utilized to increase crystallinity in the PVA network, further reducing pore sizes of the PVA network and to reduce adsorption properties of the final gel surface. Annealing can be done at temperatures ranging from, e.g., 100-200° C.; in a preferred embodiment, this step is performed submerging the dehydrated gel into a bath of mineral oil. Bulk incorporation of a polymer into a porous solid may also include an annealing process as already described above for a porous solid. Annealing may be performed after exposure of the desolvated porous solid to the mixture that has the polymers that are to be bulk incorporated. The Tg of the material may be raised or lowered dependent on the residual solvent content and/or presence of the bulk incorporated second hydrophilic polymer. As already described, the annealing process conditions may thus be adapted as to depend on temperature, time, ramp rate, and cooling rates of the substrate.

Annealing may be performed in a gas or a liquid at ambient, elevated, or low (vacuum) pressure. The liquid may be a low molecular weight polymer (up to 2000 Da) or other material (e.g., mineral oil). Examples of low molecular weight polymers are: silicone oils, glycerin, polyols, and polyethylene glycols of less than 500 Da. A useful embodiment is annealing in a bath of glycerin at, e.g., 140° C. for 1-3 hours; glycerin acts to further reduce fouling properties of the gel through interaction and neutralization of the free hydroxyl end groups of the PVA network. The annealed nanoporous material is allowed to cool, removed from the annealing bath and rinsed free of bath medium using a series of extended soaks. The product is then dehydrated to prepare for terminal sterilization.

Various types of dies may be used, e.g., longitudinal, angular, transverse and spiral extrusion heads, as well as single-polymer extrusion heads used for extruding a single polymer and multi layers extrusion heads used for simultaneous extrusion of a plurality of polymer layers or other layers. Continuous operation heads may be used, as well as cyclical. Various materials may be incorporated into, or as, a layer: for example, a reinforcing material, a fiber, a wire, a braided material, braided wire, braided plastic fibers, and so forth. Similarly, such materials may be excluded. Moreover, the porous solid may be made with a certain property, e.g., Young's modulus, tensile strength, solids content, polymer composition, porous structure, or solvent content that is known and thus measurable exclusive of various other materials. Accordingly, embodiments include materials disclosed herein that are described in terms of the materials'properties without regard to various other incorporated materials. For instance, a nanoporous solid has a certain Young's modulus that is known even if the material has a reinforcing wire that contributes further strength.

A core may be used with an extrusion die. The core may be air, water, a liquid, a solid, a non-solvent or a gas. Artisans reading this disclosure will appreciate that various extrusion processes using these various kinds of cores may be used. Cores made of polytetrafluoroethylene tubing (PTFE) are useful. In some embodiments, a core is a wire.

Multi lumen tubing has multiple channels running through its profile. These extrusions can be custom engineered to meet device designs. Multi Lumen tubing has a variable Outer Diameter (OD), numerous custom Inner Diameters (ID's), and various wall thicknesses. This tubing is available in a number of shapes; circular, oval, triangular, square, semi-circular, and crescent. These lumens can be used for guidewires, fluids, gases, wires, and various other needs. The number of lumens in multi lumen tubing is only limited by the size of the OD. In some embodiments, OD's are as large as 0.5 in., ID's can be as small as 0.002 in., and web and wall thicknesses can be as thin as 0.002 in. Tight tolerances can be maintained to +/−0.0005 in. Artisans will immediately appreciate that all ranges and values between the explicitly stated bounds are contemplated, with, e.g., any of the following being available as an upper or lower limit for an OD and/or ID: 0.002, 0.003, 0.004, 0.007, 0.01, 0.02, 0.03, 0.04, 0.05, 0.1, 0.2, 0.3, 0.4, and 0.5 in. Tolerances may be, e.g., from 0.0005 to 0.1 in. ; Artisans will immediately appreciate that all ranges and values between the explicitly stated bounds are contemplated, with, e.g., any of the following being available as an upper or lower limit: 0.0005, 0.001, 0.002, 0.003, 0.006, 0.01, 0.02, 0.03, 0.06, 0.8, 0.9, 1 in.

Braid reinforced tubing can be made in various configurations. For instance, it is possible to braid using round or flat, single or double ended wires as small as 0.001 in. Various materials can be used to make the braided reinforced tubing including stainless steel, beryllium copper, and silver, as well as monofilament polymers. The braid can be wound with various pics per inch over many thermoplastic substrates such as nylons or polyurethanes. The benefits of braided catheter shaft are its high torque-ability and kink resistance. By changing several factors during the braiding process, the characteristics of the tube can be altered to fit performance requirements. After braiding is complete, a second extrusion may be applied on top of the braided tube to encapsulate the braid and provide a smooth finish. Walls as thin as 0.007 in. can be achieved when a braid reinforced tube is required.

### Porous, Microporous, and Nanoporous Materials

Porous solid is a term used broadly herein to refer to materials having a solid phase containing open spaces and is used to describe true porous materials and also hydrogels having an open matrix structure. Some terms related to porosity are used somewhat loosely in scientific literature such that it is helpful to provide certain definitions herein. The term nanoporous material or nanoporous solid is used herein to specifically refer to a solid made with interconnected pores having a pore size of up to about 100 nm diameter. The term diameter is broad and encompasses pores of any shape, as is customary in these arts. The term microporous solid or microporous material is similarly used herein to specifically refer to a solid made with interconnected pores having a pore size of up to about 10 μm diameter. These nano-or micro-porous materials are characterized by an interconnected porous structure.

Some hydrogels, which artisans sometimes refer to as hydrogel sponges, are also true porous materials that have a continuous and solid network material filled through voids, with the voids being the pores. However, an open matrix structure found in many hydrogels is not a true porous structure and, in general, while it is convenient to refer to them as porous materials, or to use analogies to pores when characterizing diffusive or other properties, such hydrogels are not nanoporous or microporous solids as those terms are used herein. The spaces between strands of an open matrix hydrogel, and the strands of the matrix are not interconnected pores. Hydrogels are crosslinked gels that have solid-like properties without being a true solid although it is convenient herein and generally in these arts to refer to them as a solid because they are crosslinked, insoluble in solvent, and have significant mechanical strength. Hydrogels may have a high-water content, e.g., 25% w/w at EWC or more. Artisans in the hydrogel arts sometimes use the term porous, to characterize a net molecular weight cut off or to refer to spacing between strands of an open hydrogel matrix, in which case the hydrogel does not have a true porous structure and is not a nanoporous or a microporous material as those terms are used herein. The definitions of nanoporous material and microporous material as used herein also contrast with a convention that is sometimes followed wherein microporous substances are described as having pore diameters of less than 2 nm, macroporous substances have pore diameters of greater than 50 nm, and a mesoporous category lies in the middle.

The extrusion process for making the inventive materials has some advantages. The extrusion has been observed to align the polymers to a parallel orientation that contributes to high tensile strength. Having been extruded and stretched, the polymer molecules become aligned in the direction of the tube or fiber. Any tendency to return to a random orientation is prevented by the strong intermolecular forces between the molecules. Further, extrusion provides for creation of materials or devices with a high aspect ratio as compared to injection molding or other molding processes. Moreover, extrusion provides good control of dimensions such that wall thickness, placement of the lumen or lumens can be controlled. The use of high concentrations of polymers, above their melt point, in a solvent was useful for enabling extrusion. It is significant that attempts by others to use similar polymers to make high strength materials used other techniques that do not allow for extrusion, that are less efficient, and often unsuited for making actual end-user products.

For example, poly(vinyl alcohol) (PVA) was used herein to make nanoporous materials with excellent properties, especially as compared to conventionally used PVA medical materials. In fact, PVA has been used extensively throughout the medical device industry with a well-established track record of biocompatibility. PVA is a linear molecule with an extensive history as a biocompatible biomaterial. PVA hydrogels and membranes have been developed for biomedical applications such as contact lenses, artificial pancreases, hemodialysis, and synthetic vitreous humor, as well as for implantable medical materials to replace cartilage and meniscus tissues. It is an attractive material for these applications because of its biocompatibility and low protein adsorption properties resulting in low cell adhesion compared with other hydrogels.

Others have tried to improve the properties of PVA for biomedical purposes. For instance, others have experimented with a freeze/thaw process. And techniques for formation of hydrogels from PVA such as “salting out” gelation have been shown to form useful polymer hydrogels using different molecular weights and concentrations. Manipulation of Flory interactions has also been studied in the formation of PVA gels through the combination of two solutions (see U.S. Pat. Nos. 7,845,670, 8,637,063,7,619,009) for the use of PVA as an injectable in situ forming gel for repairing intervertebral disks. In general, prior processes for fabricating tough PVA materials were studied in U.S. Pat. No. 8,541,484. Methods for doing so without the use of radiation or chemical crosslinkers have also been previously studied, as shown in U.S. Pat. No. 6,231,605. None of this PVA-related work by others has resulted in the inventions that are set forth herein. Some of these other materials were useful in regards to tensile strength but were nonetheless macroporous in nature.

In contrast, processes herein provide high strength materials with a true porous structure and other useful characteristics such as an unexpectedly good combination of biocompatibility and mechanical properties. Embodiments of porous solid materials are provided that have a combination of structural features independently chosen from pore sizes, tensile strength, Young's modulus, solids concentration, crosslinking type and degree, internal alignment, hydrophilicity, and composition for the materials and further, optionally, independently selecting end-user devices or intermediate materials having a desired aspect ratio for molded shapes, a lumen, a plurality of lumens, tubes with concentrically placed lumens or a range of tolerance of thickness, or a particular medical device: each of these are further detailed herein.

Embodiments include nanoporous materials with pore diameters of 100 nm or less, or within a range of 10-100 nm; Artisans will immediately appreciate that all ranges and values between the explicitly stated bounds are contemplated, with, e.g., any of the following being available as an upper or lower limit: 1, 2, 3, 4, 5, 10, 20, 50, 60, 70 80, 90, 100 nm.

Embodiments include nanoporous materials or microporous materials with a tensile strength at break of at least about 50 MPa or from 1-300 MPa measured at EWC. Artisans will immediately appreciate that all ranges and values between the explicitly stated bounds are contemplated, with, e.g., any of the following being available as an upper or lower limit: 10, 20, 30, 40, 50, 60, 70, 100, 200, 300 MPa.

Embodiments include nanoporous materials or microporous materials with a Young's modulus strength of at least about 1 MPa or from 1-200 MPa measured at EWC. Artisans will immediately appreciate that all ranges and values between the explicitly stated bounds are contemplated, with, e.g., any of the following being available as an upper or lower limit: 5, 10, 15, 20, 25, 30, 35, 40, 50, 60, 70, 80, 90, 100, 120, 140, 160, 180, 200 MPa.

Embodiments include nanoporous materials or microporous materials or hydrogels with an elongation at break of at least about 100% or from 50-1500% measured at EWC. Artisans will immediately appreciate that all ranges and values between the explicitly stated bounds are contemplated, with, e.g., any of the following being available as an upper or lower limit: 50, 60, 70, 80, 90, 100, 200, 300, 400, 450, or 500 % (e.g., greater than or equal to 50%).

Embodiments include nanoporous materials or microporous materials or hydrogels with a solids content of at least 20% or solids from 20-90% w/w measured at EWC; Artisans will immediately appreciate that all ranges and values between the explicitly stated bounds are contemplated, with, e.g., any of the following being available as an upper or lower limit: 5, 10, 25, 30, 35, 40, 45, 50, 55, 60, 65, 70, 80, 90 % w/w percent solids. Percent solids are measured by comparing a total weight at EWC to dry weight.

The tensile strength, modulus, and elongation values may be mixed-and-matched in combinations within the ranges as guided by this disclosure.

Embodiments include nanoporous materials or microporous materials or hydrogels with physical crosslinks or covalent crosslinks or a combination thereof. Physical crosslinks are non-covalent, e.g., physical crosslinks are ionic bonds, hydrogen bonds, electrostatic bonds, Van Der Waals forces, or hydrophobic packing. The materials may be made free of covalent crosslinks, covalent crosslinkers and chemical products thereof. Chemicals can be added during processing to create covalent crosslinks, as is known in the arts of polymerization. Alternatively, the processes and materials may be free of the same.

Embodiments include nanoporous materials or microporous materials or hydrogels with an internal alignment of the polymeric structure. Alignment may be visualized using SEM images in sections taken along the direction of extrusion, i.e., longitudinally for a tube. Alignment refers to a majority horizontal chain orientation and along the length of samples (in direction of extrusion).

Embodiments include nanoporous materials or microporous materials or hydrogels with a hydrophilic surface and/or material. Materials made from polymers that are water soluble are hydrophilic. A water-soluble polymer is a polymer that is soluble in water at a concentration of at least 1 g/100 ml at 20° C. Water soluble polymers are hydrophilic. A surface is hydrophilic if a contact angle for a water droplet on the surface is less than 90 degrees (the contact angle is defined as the angle passing through the drop interior). Embodiments include hydrophilic surfaces with a contact angle from 90 to 0 degrees; Artisans will immediately appreciate that all ranges and values between the explicitly stated bounds are contemplated, with, e.g., any of the following being available as an upper or lower limit: 90, 80, 70, 60, 50, 40, 30, 20, 10, 5, 2, 0 degrees. A matrix of a material is hydrophilic relative to a solvent when the matrix is hydrophilic and a droplet of the solvent on the surface is less than 90 degrees.

Materials for use in the process and/or biomaterials may include polymers. Hydrophilic polymers are useful, e.g., one or more polymers may be selected from polyvinyl alcohol (PVA), polyvinylpyrrolidone (PVP), polyethylene glycol (PEG), polyacrylic acid (PAA), polyacrylamide, hydroxypropyl methacrylamide, polyoxazolines, polyphosphates, polyphosphazenes, poly(vinyl acetate), polypropylene glycol, Poly(N-isopropylacrylamide) (PNIPAM), polysaccharides, sulfonated hydrophilic polymers (e.g., sulfonated polyphenylene oxide, Nafion®, sulfobetaine methacrylate) and variations of the same with an added iodine (e.g., PVA-I, PVP-I), or variations with further pendent groups, copolymers of the same, and combinations of the same. Two or more hydrophilic polymers may be intermixed together to form a nanoporous material. The molecular weight of the polymer can affect the properties of the biomaterial. A higher molecular weight tends to increase strength, decrease pore size, and decrease protein adsorption. Accordingly, embodiments include a polymer or a hydrophilic polymer having a molecular weight of 40 kDa to 5000 kDa; Artisans will immediately appreciate that all ranges and values between the explicitly stated bounds are contemplated, with, e.g., any of the following being available as an upper or lower limit: 40 k, 50 k, 100 k, 125 k, 150 k, 250 k, 400 k, 500 k, 600 k 750 k, 800, 900 k, 1 million, 1.5 million, 2 million, 2.5 million, 3 million molecular weight.

The term PEG refers to all polyethylene oxides regardless of molecular weight or whether or not the polymers are terminated with a hydroxyl. Similarly, the terms PVA, PVP, and PAA are used without limitation as to terminal chemical moieties or MW ranges. References to polymers described herein include all forms of the polymers including linear polymers, branched polymers, underivatized polymers, and derivatized polymers. A branched polymer has a linear backbone and at least one branch and is thus a term that encompasses star, brush, comb, and combinations thereof. A derivatized polymer has a backbone that comprises the indicated repeating unit and one or more substitutions or pendant groups collectively referred to as derivatizing moieties. A substitution refers to a replacement of one atom with another. A pendant group is a chemical moiety attached to the polymer and may be the same or a different moiety as the polymer repeating unit. Accordingly, a reference to a polymer encompasses highly derivatized polymers and also polymers no more than 0.01-20% w/w derivatizing moieties, calculated as the total MW of such moieties compared to the total weight of the polymer. Artisans will immediately appreciate that all ranges and values between the explicitly stated bounds are contemplated, with, e.g., any of the following being available as an upper or lower limit: 0.01, 0.05, 0.1, 0.2, 0.3, 0.5, 1, 2, 3, 4, 5, 6, 7, 8, 9, 10, 15, 20% w/w.

A porous solid may be formed as a monolithic material, as a layer on another material, device, or surface, as a plurality of layers, or as one or more layers of a nanoporous material or a material that comprises a nanoporous material. Thus, for example, a plurality of layers may be extruded, with the layers being independently chosen to form one or more of: a nanoporous material, a microporous material, a hydrogel, a single-polymer material, a material having two or more polymers, and a non-nanoporous material.

The process of making the material can also affect the material properties, including the concentration of polymer in the polymeric mixture passed through a die. Starting PVA or other hydrophilic polymer concentrations may range from, e.g., 5 to 70% weight-volume (w/w) in water; generally, about 10-30% (w/w) is preferable; Artisans will immediately appreciate that all ranges and values between the explicitly stated bounds are contemplated, with, e.g., any of the following being available as an upper or lower limit: 5, 10, 15, 20, 25, 30, 35, 40, 45, 50, 55, 60, 65, 70 percent.

Processes set forth herein may be truncated at a point before polymers crosslink and are processed to become a true nanoporous material, or otherwise adapted to avoid a nanoporous structure. In general, such materials have a lesser strength and toughness and lower solids content. Such materials are generally hydrogels when hydrophilic polymers are used at relatively low solids content. Accordingly, such materials, and even hydrogels, are contemplated herein, and materials may be made that are of somewhat lesser characteristics as compared to the nanoporous materials but, nonetheless, are superior to conventional processes and materials that use the same polymers. Similarly, and as a generalization, a microporous solid would have properties that approach those of the nanoporous materials and would have a strength better than those of a hydrogel.

Artisans are accustomed to quantifying pore size distributions in materials. Nanoporous, microporous, and microporous materials are disclosed herein and control of the pore sizes of such material is demonstrated. Embodiments thus include materials that have a particular quantity or distribution of pore sizes. These can be measured at a surface, in a depth from the surface in a cross-sectional sample, or for the bulk of the material. For instance, the material pore sizes on a surface, at a depth from a surface, or in a bulk may have a percentage from 50-100% of pore diameters that fall within a range, or above or below a certain value, from 1 nm to 20 μm; Artisans will immediately appreciate that all ranges and values between the explicitly stated bounds are contemplated, e.g., 10, 20, 30, 40, 50, 60, 65, 70, 75, 80, 90, 95, 98, 99, 99.9 or 100% and 1, 10, 20, 30, 40, 50, 100, 200, 400, 500, 1000, 2000, 3000, 5000, 10000, 15000, or 20000 nm. Examples of quantitation relative to a depth are at a depth of e.g., at least, or in a range of, 1-5000 μm; Artisans will immediately appreciate that all ranges and values between the explicitly stated bounds are contemplated: 1, 2, 3, 4, 5, 10, 20, 50, 100, 250, 500, 750, 1000, 2000, 3000, 4000, or 5000 μm. For example, a surface may have a certain percentage of pores that are no more than a certain diameter or a depth or depth range may have a certain percentage of pores that are no more than a certain diameter.

Embodiments include a process for making a polymeric material comprising heating a mixture that comprises a water-soluble polymer and a solvent to a temperature above the melting point of the polymer, extruding the mixture, and cooling the mixture while removing the solvent and/or cooling the mixture while it crosslinks. When a plurality of polymers is present in a solvent, either with or without other additives, a melting point of the combined polymers in the solvent can be readily determined by the artisan, for instance by observing the mixture as it is heated and it passes from a cloudy to a markedly more translucent appearance. Further, after, or as part of, a formation process that uses the mixture, some or all of the solvent may be removed from the mixture while the cooling takes place. Embodiments include removing at least 50% w/w of the solvent in less than 60 minutes (or less than 1, 2, 5, or 10 minutes). Embodiments include removing at least 90% w/w (or at least 70% w/w or at least 80% w/w) of the solvent in less than 60 minutes (or less than 1, 2, 5, 10, or 30 minutes).

### Bulk Incorporation of Polymers Into a Porous Solid

A porous material may be exposed to a mixture comprising solvated polymers (for bulk incorporated polymers) to draw them into the pores when the porous matrix is desolvated. The solvent of the mixture has an affinity for the matrix and is drawn in as the matrix imbibes the solvent. The solvent in the mixture with the bulk incorporated polymers can be chosen to have an affinity for the matrix so that it is imbibed into the desolvated matrix but does not have to be the same as the solvent in the matrix. In general, a hydrophilic solvent in the mixture will be imbibed into a hydrophilic porous matrix that is at least partially desolvated and contains a hydrophilic solvent, and an artisan can adjust the various solvents as needed to create suitable conditions when the goal of bulk incorporation is intended.

A hydrophilic solvent is a solvent that is freely miscible with water or is present at a concentration in the mixture wherein it is freely miscible with water, at 20° C.

Desolvated means that the matrix is free of solvents, e.g., completely dry, or is below an EWC of the matrix relative to the solvent it contains. If the solvent in the matrix is not water, the EWC can be calculated for the material based on measurements in the solvent, i.e., the term EWC can be used for solvents that are not water in the appropriate context. For instance, a hydrophilic matrix might be solvated in an aqueous solution of an alcohol and would have an EWC for that solvent. Embodiments include an amount of desolvation of a porous solid from 1-100, Artisans will immediately appreciate that all ranges and values between the explicitly stated bounds are contemplated: 1, 5, 10, 15, 20, 33, 40, 50, 60, 70, 80, 90, 95, 99, 100% w/w referring to the total weight of solvent that can be removed.

Without being bound to a particular theory, it is believed that porous materials can be desolvated (dehydrated in the case of water being the solvent in the porous material) and exposed to polymers in a solution that resolvates the porous material so that the polymers are drawn into the pores. The polymers then form physical bonds with the matrix material that defines the pores and are, for practical purposes, permanently incorporated into the bulk of the materials, both by at least partially filling the pores and by physical bonding with the matrix. Alternatively, or additionally, the polymers have a hydrodynamic radius that causes the polymer to present a diameter that exceeds the pores'opening diameter so that the polymer is permanently incorporated into the pores of the material, especially when the material is to be used in water or physiological solution. In general, if the bulk-incorporated polymer is solvated in a polymer that wets the pores of the porous solid, the polymer can be drawn into pores of the matrix as it is resolvated. When a hydrophilic porous matrix is below an EWC of the matrix, the mixture that contains the polymers for bulk incorporation is drawn in because the solvent for the polymers is matched to the matrix material, e.g., wets the pores of the material. For instance, a hydrophilic solvent will normally wet the pores of a hydrophilic matrix.

A material that comprises a porous matrix of polymers joined by noncovalent bonds is a preferred embodiment, since these materials can be made with a high degree of control over pore sizes and material properties, including a choice of nanoporous, microporous, or other characteristic pore sizes. The matrix may comprise physically crosslinked water-soluble polymers that define the pores. A solids concentration of these water-soluble polymers may be at least 33% w/w of the matrix at an equilibrium water content (EWC) of the matrix, although other concentrations may also be used.

Accordingly, an embodiment of a process of incorporating polymers in a porous material comprises providing a material comprising a porous, hydrophilic matrix that comprises one or more water soluble polymers (also referred to herein as matrix polymers) crosslinked with each other to form the matrix. The material with the matrix is exposed to a mixture comprising one or more polymers (also referred to as bulk incorporated polymers, preferably with the polymers being water soluble, with the mixture also being referred to as a conditioning mixture or bulk incorporating mixture) solvated in a solvent, wherein the matrix is below the EWC before being exposed to the mixture and is hydrophilic relative to the solvent. The material, before exposure to the mixture with the bulk incorporated polymers, is desolvated.

In some embodiments, bulk incorporation processes create an outer zone wherein the pores are filled, an intermediate zone where most of the pores are filled or are mostly filled, and an inner zone where there is little or no penetration of the polymers. Bulk incorporation not only modifies pores at a surface but also below the surface, e.g., at least, or in a range of, 1-5000 μm; Artisans will immediately appreciate that all ranges and values between the explicitly stated bounds are contemplated, e.g., 1, 2, 3, 4, 5, 10,20, 30, 40, 50, 75, 100, 250, 500, 750, 1000, 2000, 3000, 4000, or 5000 μm. The percentage of pores that have polymer may be assayed as already described and penetration graded by a cut-off of a percentage, e.g., a first zone having 100% filling of pores, a second zone with 50% pores filled, a third zone with 0% pores filled.

Bulk incorporation processes may be made with porous matrices that are made of water soluble polymers and may be made without hydrophobic domains in the polymers, e.g., a matrix made only of PVA. The polymers may form the matrix with physical crosslinks. Accordingly, embodiments include materials comprising matrices that are free of hydrophobic domains or that are made with water soluble polymers that are free of hydrophobic domains or that are free of any polymer that is not water soluble. Some hydrophobic domains can be tolerated, however, when making a hydrophilic matrix with water soluble polymers having physical crosslinks without disrupting the matrix formed thereby. Embodiments of the invention include a hydrophobic content of polymers that form a porous matrix of 0, 1, 2, 3, 4, 5, 7, 8, 9, 10, 11, 12, or 15% w/w.

A porous matrix consisting essentially of water soluble polymers refers to a content of up to 3% w/w of the polymers that crosslink to form the matrix. RO agents such as salts are not polymers that crosslink to form the matrix. A porous matrix consisting essentially of physically crosslinked polymers refers to a matrix that is free of agents that make covalent bonds between the polymers, or has a small amount of such agents so that no more than about 6% of the polymers (referring to polymer number) are crosslinked to each other with such agents, e.g., wherein a stoichiometric ratio of polymer number to a bifunctional crosslinker is at least 100:3. A matrix that is essentially free of covalent bonds similarly is made with polymers crosslinked with no more than about 6% of the polymers (by number) are not covalently crosslinked. The number of covalent bonds in a matrix may similarly be limited to a stoichiometric ratio of 100:3 to 100:100, e.g., 100 to any of 3, 5, 10, 15, 20, 25, 30, 40, 50, 60, 70, 80, 90, or 100 by number. For instance, hydrogels made by free radical polymerization typically have 100% of the polymers attached to each other by covalent bonds, which is a 100:100 stoichiometric ratio of polymers: covalent bonds.

As stated elsewhere, a porous solid can be made with a controlled pore diameter range and may be made to provide a matrix that has no pores larger than a particular diameter. Diameters may be measured in an appropriate context, e.g., at EWC in distilled water. Embodiments thus include polymers entrapped in a porous matrix that is free of pores that are larger than 1-5000 μm; Artisans will immediately appreciate that all ranges and values between the explicitly stated bounds are contemplated, e.g., 1, 2, 3, 4, 5, 10, 15, 20, 25, 30, 50, 100, 200, 250, 300, 400, 500, 750, 1000, 2000, 3000, 4000, or 5000 μm.

A porous solid can have other materials present as described elsewhere herein, e.g., radiopaque (RO) agents that are additional to the matrix but are not part of the matrix. RO agents typically contribute little to the crosslinking that provides the strength of the matrix. Similarly, other materials can be present in the matrix without being part of the matrix, e.g., wires and reinforcing materials. It can be appreciated that a matrix made with physical crosslinks is one type of matrix that can be made with materials that define pores that have diameters and is in contrast to hydrogels having polymer strands that are generally separated from each other and are connected in a mesh network structure, e.g., as typically formed using free radical polymerization or by reaction of monomers/polymers that are in solution. Such mesh networks would generally not be expected to stably incorporate polymers in their pores without covalent bonding using a polymer-imbibing process. Porous materials are described in detail herein and these may be freely chosen, as guided by the disclosure herein, for use with bulk incorporated polymers. The porous material may be chosen with bulk properties as described herein.

The bulk incorporated polymers may be polymers described elsewhere herein for porous solids. Examples are water-soluble polymers. The water soluble polymers may be, for example, polyvinyl alcohol (PVA), polyvinylpyrrolidone (PVP), polyethylene glycol (PEG), polyacrylic acid (PAA), polyacrylamide, hydroxypropyl methacrylamide, polyoxazolines, polyphosphates, polyphosphazenes, poly(vinyl acetate), polypropylene glycol, Poly(N-isopropylacrylamide) (PNIPAM), polysaccharides, sulfonated hydrophilic polymers (e.g., sulfonated polyphenylene oxide, Nafion®, sulfobetaine methacrylate) and variations of the same with an added iodine (e.g., PVA-I, PVP-I), or variations with further pendent groups, copolymers of the same, and combinations of the same. The mixture may comprise one or more polymers, meaning polymers of different chemical compositions, such as PVA and PEG. The term “a polymer” refers to one or more polymers.

The solubility of a water-soluble polymer for a porous matrix or for bulk incorporation may be chosen as, e.g., at least 1, 2, 5, or 10 g/100 ml in water at 20° C. Polymers may be chosen to be linear or branched. Embodiments include a polymer or a hydrophilic polymer having a molecular weight of, e.g., 40 k to 5000 k Daltons; Artisans will immediately appreciate that all ranges and values between the explicitly stated bounds are contemplated, with, e.g., any of the following being available as an upper or lower limit: 40 k, 50 k, 100 k, 125k, 150 k, 250 k, 400 k, 500 k, 600 k 750 k, 800, 900 k, 1 million, 1.5 million, 2 million, 2.5 million, 3 million molecular weight. The molecular weight of the polymer can be chosen in light of the pore sizes available in the porous solid. Nanoporous or microporous materials are preferred.

The bulk incorporated polymers may be chosen to be the same as polymers that form the porous matrix, to be the same as at least one of the polymers that make up the matrix, or to be different.

The bulk incorporated polymer concentrations in the mixture may be, referring to the mixture at the start of the process, any concentration wherein the polymers go into solution, bearing in mind that polymer that is not in solution, or other non-solvated materials, are not destined to enter pores. In some embodiments, concentrations are 1-50% w/w; Artisans will immediately appreciate that all ranges and values between the explicitly stated bounds are contemplated, e.g., 1, 2, 3, 4, 5, 10, 15, 20, 25, 30, 33, 35, 40, 50% w/w.

Solvent for the mixture is chosen as appropriate to solvate the polymer and to provide a solvent that will be imbibed by the porous solid. Hydrophilic solvents are generally preferable for a hydrophilic matrix. Solvents may be water, organic, or aqueous, or free of the same, e.g., free of organic solvent. In some embodiments, concentrations of water are 0-99, e.g., 0, 5, 10, 15, 20, 25, 30, 40, 50, 60, 70, 80, 90, 95, or 99 w/w%.

A temperature of the conditioning mixture is not to exceed a melting temperature of the porous solid matrix. Temperatures ranges may be, for example, from 10-100° C., e.g., 10, 20, 30, 37, 40, 50, 60, 70, 80, or 90° C.

Exposure times are preferably for a duration of time required for a porous solid to reach EWC in the mixture. Duration of time may comprise, in some embodiments, 2, 4, 6, 8, 10, 12, 16, 20, 24, and 48 hours. Agitation and temperature may be manipulated to affect a time of exposure, e.g., to accelerate achieving EWC or to control viscosity of the mixture. Salt and/or osmotic content may be adjusted as helpful, e.g., for solubility, viscosity, and/or EWC.

The Examples provide guidance in regards to salt concentration for a conditioning mixture. Examples of salt concentration are from 0.1 to 2% w/w. In general, a single charge cation with a smaller atomic radius has a greater penetration into a depth of a porous solid, whereas a larger cation reduces penetration. Examples of salts are those with a single cation, divalent cation, or other cation, e.g., a salt of sodium, potassium, lithium, copper, quaternary ammonium (NR*, where R is a hydrogen, alkyl, or aryl group), magnesium, calcium, copper, iron, or zinc. In general, a physiological pH using a buffer was useful for the mixture. A pH may be adjusted to increase or decrease penetration into a matrix, and the solvent may include or omit buffering salts. Examples of pH are from 4-10, e.g., 4, 5, 6, 7, 8, 9, or 10.

A viscosity of a conditioning mixture, referring to a water-soluble polymer and solvent, is affected by: pH (higher pH, higher viscosity), polymer concentration and/or molecular weight, and polymer branching, with increases in any of these generally leading to a higher viscosity. In general, a higher viscosity reduces penetration of the bulk incorporating polymers into a porous solid. An embodiment is a porous material comprising water soluble polymers entrapped in pores of a porous matrix. The matrix may comprise physically crosslinked water-soluble polymers that are crosslinked with each other to form the matrix and define the pores. The matrix may have features as disclosed herein, e.g., polymer content, weight percentage of polymers, strength, Young's modulus, degree of coverage, pore sizes, and so forth.

Surface coverage of the water-soluble polymers in a porous matrix may be complete. Complete coverage under SEM conditions wherein no pores of the underlying surface are visible indicates coverage at EWC. A degree of coverage may be less than 100%, e.g., from 50-100%; Artisans will immediately appreciate that all ranges and values between the explicitly stated bounds are contemplated, e.g., 50, 60, 70., 80, 90, 95, 98, 99, 99.9, or 100%.

Bulk incorporation can decrease physical properties of a porous solid. Embodiments thus include a porous solid, e.g., one as disclosed herein, with a Young's modulus and/or tensile strength that is from 1-20% less as a result of being conditioned with a water-soluble polymer as compared to the same material that has not been conditioned with a water-soluble polymer; Artisans will immediately appreciate that all ranges and values between the explicitly stated bounds are contemplated, e.g., 1, 2, 3, 4, 5, 7, 9, 10, 12, 15, or 20%.

### Products

Products, including end-user or intermediate products, or materials, may be made that have an aspect ratio as desired, e.g., at least 3:1, referring to materials set forth herein including nanoporous materials, microporous materials, and hydrogels. The aspect ratio increases as the polymeric tube increases in length and decreases in width. Artisans will immediately appreciate that all ranges and values between the explicitly stated bounds are contemplated, with, e.g., any of the following being available as an upper or lower limit: 3:1, 4:1, 5:1, 6:1., 7:1, 8:1., 9:1, 10:1, 50:1, 100:1, 1000:1. A high aspect ratio is highly advantageous for certain polymeric tubes, e.g., many types of catheters. In principle, a thin tube could be continuously extruded without limitation as to length. Such polymeric tubes include, e.g., tubes, rods, cylinders, and cross-sections with square, polygonal, or round profiles. One or more lumens may be provided in any of the same. The polymeric tube may be made of a single material, essentially a single material, or with a plurality of materials including the various layers already discussed, or a reinforcing material, a fiber, a wire, a braided material, braided wire, braided plastic fibers.

The extrusion process, in particular, provides for concentric placement of a lumen; concentric is in contrast to eccentric meaning the lumen is off-center. In the case of a plurality of lumens, the lumens may be placed so that the lumens are symmetrically placed: the symmetry is in contrast to an eccentric placement of the lumens that is a result of a poorly controlled process. Embodiments include the aforementioned polymeric tubes with an aspect ratio of at least 3:1 with lumens that are positioned without eccentricity or one lumen that is concentric with the longitudinal axis of the polymeric tube.

The porous solids such as the nanoporous materials, microporous materials, and strong hydrogels may be used to make catheters or medical fibers. These may be made with bulk incorporated polymers and may have the various features described for the same. Examples of catheters are central venous, peripherally inserted central, midline, peripheral, tunneled, dialysis access, hemodialysis, vascular access port, peritoneal dialysis, urinary, neurological, peritoneal, intra-aortic balloon pump, diagnostic, interventional, drug delivery, etc.), shunts, wound drains (external including ventricular, ventriculoperitoneal, and lumboperitoneal), and infusion ports. The porous solids may be used to make implantable devices, including fully implantable and percutaneously implanted, either permanent or temporary. The porous solid materials may be used to make blood-contacting devices or devices that contact bodily fluids, including ex vivo and/or in vivo devices, and including blood contacting implants. Examples of such devices include drug delivery devices (e.g., insulin pump), tubing, contraceptive devices, feminine hygiene, endoscopes, grafts (including small diameter <6 mm), pacemakers, implantable cardioverter-defibrillators, cardiac resynchronization devices, cardiovascular device leads, ventricular assist devices, catheters (including cochlear implants, endotracheal tubes, tracheostomy tubes, drug delivery ports and tubing, implantable sensors (intravascular, transdermal, intracranial), ventilator pumps, and ophthalmic devices including drug delivery systems. Catheters can comprise a tubular nanoporous material with a fastener to cooperate with other devices, e.g., luer fasteners or fittings. Radiopaque agents may be added to the materials, fibers, or devices. The term radiopaque agent refers to agents commonly used in the medical device industry to add radiopacity to materials, e.g., barium sulfate, bismuth, or tungsten. RO agents may be incorporated at, e.g., from 5-50% w/w pf the total solids weight, e.g., 5, 10, 20, 30, 40, or 50%.

Medical fibers made with porous solid materials include applications such as sutures, yarns, medical textiles, braids, mesh, knitted or woven mesh, nonwoven fabrics, and devices based on the same. The fibers are strong and pliable. Materials may be made with these fibers so that they are resistant to fatigue and abrasion.

It should further be understood that in some embodiments, a device or article provided herein comprises a combination one or more of the above-mentioned devices or articles. For example, in some embodiments a device comprises a catheter associated with (e.g., containing, comprising, adjacent, directly adjacent) a balloon (e.g., a catheter comprising a polymeric tube, at least a portion of which has been inflated into to form the balloon). A device comprising a catheter and a balloon may, in some embodiments, be formed by exposing a portion of a polymeric tube to a thermal reservoir (e.g., a bath) to selectively soften the portion of the polymeric tube. The softened portion may then be inflated to form the balloon (e.g., by increasing air pressure within the tube relative to external air pressure). In some embodiments, the balloon may then be treated to alter its mechanical properties as desired. For example, the balloon may be stiffened, e.g., by exposing the balloon to a non-wetting thermal reservoir such as an oil bath to remove excess water.

In an exemplary embodiment, the method comprises administering, into an external orifice of a subject, a device comprising polymeric tube with a body portion that comprises a polymeric material comprising a water-soluble polymer and a biologically active agent associated with the polymeric material. In some embodiments, the polymeric tube has an aspect ratio of greater than or equal to 3:1. In some embodiments, the biologically active agent is distributed within the polymeric material substantially homogeneously. In some embodiments, the biologically active agent is distributed within the polymeric material non-homogeneously (i.e., on one or more surfaces of the polymeric material). In some embodiments, administration of the polymeric tube (e.g., polymeric tube 10 of FIG. 5A, polymeric tube 12 of FIG. 5B, polymeric tube 14 of FIG. 5C) does not comprise the use of a sheath introducer. The polymeric material is substantially non-thrombogenic, the polymeric material has a water content of less than 5 w/w% and greater than or equal to 0.1 w/w% in the first configuration (e.g., a water content less than the equilibrium water content state, such as the dehydrated state), and the polymeric material is configured to swell in an amount greater than or equal to 5 w/w% and less than or equal to 50 w/w% from a first configuration (e.g., a water content less than the equilibrium water content state, such as the dehydrated state) to a second configuration (e.g., an equilibrium water content state) in less than or equal to 60 minutes (e.g., less than or equal to 10 minutes, less than or equal to 5 minutes, less than or equal to 1 minute, or less than or equal to 10 seconds).

### Methods of Treatment

In some aspects, methods of treating a subject are described. In some embodiments, the method comprises administering, into an orifice of a subject, a polymeric tube, article, or device described herein (e.g., any embodiment of a polymeric tube, article, or device described herein or combinations thereof).

In some embodiments, the method comprises swelling the polymeric material as described herein. For example, in some embodiments, the method comprises swelling a polymeric tube and/or polymeric material in an amount greater than or equal to 2 w/w%, greater than or equal to 3 w/w%, greater than or equal to 4 w/w%, greater than or equal to 5 w/w%, greater than or equal to 10 w/w%, greater than or equal to 15 w/w%, greater than or equal to 20 w/w%, greater than or equal to 25 w/w%, greater than or equal to 30 w/w%, greater than or equal to 35 w/w%, greater than or equal to 40 w/w%, or greater than or equal to 45 w/w%, for example, from a first configuration (e.g., a water content less than the equilibrium water content state, such as the dehydrated state) to a second configuration (e.g., the equilibrium water content state). In some embodiments, the method comprises swelling a polymeric tube and/or polymeric material in an amount less than or equal to 50 w/w%, less than or equal to 45 w/w%, less than or equal to 40 w/w%, less than or equal to 35 w/w%, less than or equal to 30 w/w%, less than or equal to 25 w/w%, less than or equal to 20 w/w%, less than or equal to 15 w/w%, or less than or equal to 10 w/w%,, for example, from a first configuration (e.g., a water content less than the equilibrium water content state, such as the dehydrated state) to a second configuration (e.g., the equilibrium water content state). Combinations of these ranges are also possible (e.g., greater than or equal to 5 w/w% and less than or equal to 40 w/w%).

In some embodiments, the method comprises swelling the polymeric material to the equilibrium water content state. In some embodiments, the method comprises swelling the polymeric material to the equilibrium water content state over a duration of time. In some embodiments the duration of time is less than or equal to 60 minutes (e.g., less than or equal to 10 minutes, less than or equal to 5 minutes, less than or equal to 1 minute, less than or equal to 30 seconds, or less than or equal to 10 seconds).

In some embodiments, the method comprises swelling the polymeric material at a given temperature. In some embodiments, the temperature is greater than or equal to 4° C., greater than or equal to 10° C., greater than or equal to 16° C., greater than or equal to 20° C., greater than or equal to 25° C., or greater than or equal to 30° C. In some embodiments, the temperature is less than or equal to 40° C. less than or equal to 30° C., less than or equal to 25° C., less than or equal to 20° C., less than or equal to 16° C., or less than or equal to 10° C. Combinations of these ranges are also possible (e.g., 20 C.-40° C.).

In some embodiments, the method comprises swelling the polymeric material such that the inner diameter and/or outer diameter increase by a larger percentage than the percentage increase in length (as described herein). For example, in some embodiments, the method comprises swelling the polymeric material such that the inner diameter and/or outer diameter increases by 1-20% while the length increases by 0.1-19%.

In some embodiments, the swelling occurs after administration. In some embodiments, the swelling of the polymeric material after administration into an orifice of a subject closes an opening of that orifice. For example, in some embodiments, the swelling of the polymeric material results in an increase in size to a dimension greater than or equal to the size of the orifice to which it is inserted. In some embodiments, the orifice is a wound. In some embodiments, the swelling of the polymeric material causes hemostasis. For example, in some embodiments, a subject (e.g., a human) may have an orifice (e.g., a wound) that has a maximum cross-sectional diameter of A and that is bleeding, and a polymeric tube described herein with a maximum outer cross-sectional diameter smaller than A may be administered into the orifice. In some embodiments, the maximum outer cross-sectional diameter of the polymeric tube may then swell to a dimension greater than or equal to A, such that the orifice is closed. In some embodiments, this may result in hemostasis.

In some embodiments, the swelling occurs before administration. In some embodiments, the swelling comprises rehydrating the polymeric tube for a duration of time. In some embodiments, the duration of time is less than or equal to 60 minutes (e.g., less than or equal to 10 minutes, less than or equal to 5 minutes, less than or equal to 1 minute, or less than or equal to 10 seconds). In some embodiments, rehydrating the polymeric tube comprises use of rehydration media. In some embodiments, the rehydration media comprises water, lactated Ringer's solution (LRS), dextrose (D5W), phosphate buffered saline (PBS), Hanks'Balanced Salt Solution (HBSS), and/or isotonic salt solutions.

### Kits

In some aspects, kits are described. The kit may comprise any suitable articles described herein. In some embodiments, the kit comprises a polymeric tube (e.g., any embodiment of a polymeric tube described herein or combinations thereof).

In some embodiments, the kit further comprises a humidity control sponge. The humidity control sponge may comprise a woven, non-woven, porous, and/or solid material comprising water and/or hydration media. In some embodiments, the humidity control sponge is a porous cellulose non-woven fabric swollen with water. In some embodiments, the humidity control sponge further comprises an antiseptic or anti-infective agent (e.g., bleach, sodium hypochlorite, peroxides, and/or peracetic acid).

In some embodiments, the kit further comprises hydration media. Non-limiting examples of suitable hydration media include water, lactated Ringer's solution (LRS), dextrose (D5W), phosphate buffered saline (PBS), Hanks'Balanced Salt Solution (HBSS), and/or isotonic salt solutions. In some embodiments, a sufficient volume of hydration media required to fully hydrate the polymeric tube to EWC is included in the kit. In some embodiments, the hydration media is stored in a vessel, fluid reservoir, tube, syringe, bag, fluid pump, and/or packet. In some embodiments, the hydration media is sterilized. In some embodiments, the hydration media is buffered at or near physiological pH (e.g., 6.8-7.8).

In some embodiments, the kit is sterile. In some embodiments, the kit is sealed.

In some embodiments, the kit includes instructions for use. In some embodiments, the instructions for use describe a method of treatment described herein.

In some embodiments, the kit comprises packaging. In some embodiments, the packaging comprises a flexible container. In some embodiments, the flexible container comprises flashspun high-density polyethylene fibers. In some embodiments, the packaging comprises a tray into which the polymeric tube can be positioned for shipment.

### Further Definitions

The term medically acceptable refers to a material that is highly purified to be free of contaminants and is nontoxic. The term consists essentially of, as used in the context of a biomaterial or medical device, refers to a material or device that has no more than 3% w/w of other materials or components and said 3% does not make the device unsuited to intended medical uses. Equilibrium water content (EWC) is a term that refers to the water content of a material when the wet weight of the material has become constant, and before the material degrades. In general, materials with a high solids content have been observed to be at equilibrium water content at 24-48 hours. For purposes of measuring EWC, distilled water is used unless otherwise specified.

The term w/v refers to weight per volume e.g., g/L or mg/mL. The terms biomaterial and biomedical material are used interchangeably herein and encompass biomedically acceptable materials directed to a use in the biomedical arts, for example, as implants, catheters, blood-contacting materials, tissue-contacting materials, diagnostic assays, medical kits, tissue sample processing, or other medical purposes. Moreover, while the materials are suited for biomedical uses, they are not limited to the same and may be created as general-purpose materials. A physiological saline refers to a phosphate buffered solution with a pH of 7-7.4 and a human physiological osmolarity at 37° C.

The term molecular weight (MW) is measured in g/mol. The MW of a polymer refers to a weight average MW unless otherwise stated. When the polymer is part of a porous solid, the term MW refers to the polymer before it is crosslinked. When a distance between crosslinks is specified, it is the weight average MW between crosslinks unless otherwise indicated. The abbreviation k stands for thousand, M stands for million, and G stands for billion such that 50 k MW refers to 50,000 MW. Daltons is also a unit of MW and likewise refers to a weight average when used for a polymer.

Publications, journal devices, patents and patent applications referenced herein are hereby incorporated herein for all purposes, with the instant specification controlling in case of conflict. Features of embodiments set forth herein may be mixed and matched as guided by the need to make an operable process or product.

As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition.

As used herein, when a component is referred to as being “adjacent” another component, it can be directly adjacent to (e.g., in contact with) the component, or one or more intervening components also may be present. A component that is “directly adjacent”another component means that no intervening component(s) is present.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the self-righting device.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %”is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a polymeric tube comprising a lumen, a first portion and a second portion;
wherein:
the first portion at least partially disposed between the second portion and the lumen,
the first portion has a first Young's elastic modulus, the second portion has a second Young's elastic modulus, and the first Young's elastic modulus is different than the second Young's elastic modulus, and
wherein one or more of the following hold:
(i) the polymeric tube comprises at least one water soluble polymer,
(ii) the polymeric tube is free of covalent crosslinking agents,
(iii) the polymeric tube has a solids content of 33% to 90% w/w at equilibrium water content (EWC),
(iv) the first portion and/or the second portion has a Young's modulus of 5-100 MPa at equilibrium water content (EWC), and
(v) the polymeric tube comprises a plurality of interconnected pores, the pores having an average diameter of less than or equal to 1 micron.

2. An article, comprising:
a polymeric tube comprising a lumen, a first portion and a second portion;
wherein:
a thickness of the first portion perpendicular to the lumen differs from a thickness of the second portion perpendicular to the lumen,
the first portion has a first Young's elastic modulus, the second portion has a second Young's elastic modulus, and the first Young's elastic modulus is different than the second Young's elastic modulus, and
wherein one or more of the following hold:
(i) the polymeric tube comprises at least one water soluble polymer,
(ii) the polymeric tube is free of covalent crosslinking agents,
(iii) the polymeric tube has a solids content of 33% to 90% w/w at equilibrium water content (EWC),
(iv) the first portion and/or the second portion has a Young's modulus of 5-100 MPa at equilibrium water content (EWC), and
(v) the polymeric tube comprises a plurality of interconnected pores, the pores having an average diameter of less than or equal to 1 micron.

3-4. (canceled)

5. An article as in claim 1, wherein the polymeric tube comprises a gradient of Young's elastic modulus along a cross-sectional dimension of the polymeric tube.

6. An article as in claim 5, wherein the cross-sectional dimension is the length of the polymeric tube.

7. An article as in claim 5, wherein the cross-sectional dimension is the diameter of the polymeric tube.

8. An article as in claim 1, wherein the first portion and the second portion are discrete portions of the polymeric tube.

9. An article as in claim 1, wherein the article comprises a hemodialysis catheter, a peripherally inserted central catheter (PICC), a central venous catheter, a midline catheter, a peripheral catheter, a urinary catheter, a neurological catheter, a peritoneal catheter, and/or an infusion port.

10. An article as in claim 1, wherein the first portion is a proximal portion of the article.

11. An article as in claim 1, wherein the first Young's elastic modulus is less than the second Young's elastic modulus.

12-13. (canceled)

14. An article as in claim 1, wherein the at least one water soluble polymer comprises poly(vinyl alcohol), poly(acrylic acid), polyethylene glycol, and/or poly(vinyl pyrrolidone).

15. An article as in claim 1, wherein the second portion has a swelling of 50% w/w or less at EWC.

16. An article as in claim 1, wherein the swellability of the first portion and the second portion are different.

17. An article as in claim 1, wherein the polymeric tube further comprises a therapeutic agent.

18. An article as in claim 17, wherein a first release rate of the therapeutic agent from the first portion is different than a second release rate of the therapeutic agent from the second portion.

19. An article as in claim 1, wherein a Young's elastic modulus of the polymeric tube varies along a gradient between the first portion and the second portion.

20. An article as in claim 19, wherein the average gradient of the Young's elastic modulus between the first portion and the second portion is greater than or equal to 0.1 MPa/cm and 500 MPa/cm.

21-22. (canceled)

23. An article as in claim 1, wherein a thickness of the first portion exceeds a thickness of the second portion and the Young's elastic modulus of the first portion is less than the Young's elastic modulus of the second portion.

24-30. (canceled)

31. A method of forming a swellable article, the method comprising:
with a polymeric mixture comprising at least one water soluble polymer and a solvent, the polymeric mixture having a concentration of at least 10% w/w of the at least one water soluble polymer, performing the steps of:
heating the polymeric mixture to achieve a first temperature above the melting point of the polymeric mixture,
extruding the polymeric mixture as a tube, and
(a) inducing a thermal gradient in the tube to produce a gradient in a mechanical property of the tube; or
(b) exposing a first portion of the tube to a first solvent for a duration such that a second portion of the tube is not exposed to the solvent for the duration to produce a gradient in a mechanical property of the tube.

32-34. (canceled)

35. A method as in claim 31, further comprising removing the solvent from the tube at a second temperature above a freezing point of the solvent until the tube is a porous solid, with the tube being a porous solid that comprises the at least one water soluble polymer, and the porous solid tube is made without covalent crosslinking agents that form covalent crosslinks between polymers extruded to make the porous solid.

36. (canceled)

37. A method as in claim 31, wherein the polymeric mixture is never heated above a boiling point of the polymeric mixture, and the polymeric mixture is extruded at temperatures below a melting point of the polymeric mixture.

38. (canceled)
